(19) United States
(12) Patent Application Publication
(10) Pub. No.: US 2004/0127575 A1
(43) Pub. Date: Jul. 1, 2004

(54) Method for counteracting a pathologic change in the beta-adrenergic pathway

(76) Inventors: Feng Ying, Sunnyvale, CA (US); Linda S. Higgins, Palo Alto, CA (US); Ann M. Kapoun, Mountain View, CA (US); David Y. Liu, Palo Alto, CA (US); George F. Schreiner, Los Altos, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/718,948

(22) Filed: Nov. 20, 2003

Related U.S. Application Data
(60) Provisional application No. 60/429,046, filed on Nov. 22, 2002.
(60) Provisional application No. 60/504,585, filed on Sep. 18, 2003.

(51) Int. Cl. A61K 31/137

(52) U.S. Cl. 514/651

(57) ABSTRACT

The invention concerns methods for modulating the β-adrenergic pathway. In particular, the invention concerns methods for counteracting a pathologic change, such as, for example, a loss in β-adrenergic sensitivity, in the β-adrenergic signal transduction pathway by administering an effective amount of a compound capable of inhibiting TGF-β signaling through a TGF-β receptor.

### CROSS-REFERENCE TO RELATED APPLICATIONS

This is a non-provisional application filed under 37 C.F.R. 1.53(b), claiming priority under 35 U.S.C. § 119(e) to Provisional Application Serial No. 60/429,046, filed on Nov. 22, 2002 and Provisional Application Serial No. 60/504,585, filed Sep. 18, 2003.

### BACKGROUND OF THE INVENTION

1. Field of the Invention

The present invention concerns methods for modulating the β-adrenergic pathway. In particular, the invention concerns methods for counteracting a pathologic change, such as, for example, a loss in β-adrenergic sensitivity, in the β-adrenergic signal transduction pathway.

2. Description of the Related Art

Transforming Growth Factor-Beta

Transforming growth factor-beta (TGF-β) denotes a family of proteins, TGF-β1, TGF-β2, and TGF-β3, which are pleiotropic modulators of cell growth and differentiation, embryonic and bone development, extracellular matrix formation, hematopoiesis, immune and inflammatory responses (Roberts and Sporn Handbook of Experimental Pharmacology (1990) 95:419-58; Massague et al. Ann Rev Cell Biol (1990) 6:597-646). Other members of this superfamily include activin, inhibin, bone morphogenic protein, and Mullerian inhibiting substance. TGF-β initiates intracellular signaling pathways leading ultimately to the expression of genes that regulate the cell cycle, control proliferative responses, or relate to extracellular matrix proteins that mediate outside-in cell signaling, cell adhesion, migration and intercellular communication.

TGF-β, including TGF-β1, -β2 and -β3, exerts its biological activities through a receptor system including the type I and type II single transmembrane TGF-β receptors (also referred to as receptor subunits) with intracellular serine-threonine kinase domains, that signal through the Smad family of transcriptional regulators. Binding of TGF-β to the extracellular domain of the type II receptor induces phosphorylation and activation of the type I receptor (TGFβ-RI) by the type II receptor (TGFβ-RII). The activated TGFβ-RI phosphorylates a receptor-associated co-transcription factor Smad2/Smad3, thereby releasing it into the cytoplasm, where it binds to Smad4. The Smad complex translocates into the nucleus, associates with a DNA-binding cofactor, such as Fast-1, binds to enhancer regions of specific genes, and activates transcription. The expression of these genes leads to the synthesis of cell cycle regulators that control proliferative responses or extracellular matrix proteins that mediate outside-in cell signaling, cell adhesion, migration, and intracellular communication. Other signaling pathways like the MAP kinase-ERK cascade are also activated by TGF-β signaling. For review, see, e.g. Whitman, Genes Dev. 12:2445-62 (1998); and Miyazono et al., Adv. Immunol. 75:111-57 (2000), which are expressly incorporated herein by reference. Further information about the TGF-β signaling pathway can be found, for example, in the following publications: Attisano et al., “Signal transduction by the TGF-β superfamily” Science 296:1646-7 (2002); Bottinger and Bitzer, “TGF-β signaling in renal disease” Am. Soc. Nephrol. 13:2600-2610 (2002); Topper, J. N., “TGF-β in the cardiovascular system: molecular mechanisms of a context-specific growth factor” Trends Cardiovasc. Med. 10:132-7 (2000), review; Itoh et al., “Signaling of transforming growth factor-β family” Eur. J. Biochem. 267:6954-67 (2000), review.

TGF-β-induced down-regulation of beta-adrenergic receptors has been observed in cardiac fibroblasts, and in bronchial smooth muscle cells, glioma cells, and renal epithelial cells. For example, TGF-β1 has been shown to induce β2-adrenoreceptor desensitization through the alteration in adenylyl cyclase activity and down-regulation of β2-adrenoreceptor mRNA and protein through the reduction in the rate of β2-adrenoreceptor gene transcription.

Beta-Adrenergic Receptors

The beta-adrenergic receptors (βARs) belong to a large family of seven transmembrane-domain receptors that couple and signal through guanine nucleotide binding proteins (G-proteins) coupled to adenylyl cyclase (AC). βARs are classified into β1, β2, and β3 subgroups, which show distinctly different expression patterns. β1AR is mainly expressed in cardiac tissue, β2AR, is highly expressed in airway smooth muscle tissue, and also in cardiac and other tissues; β3 is expressed mainly in adipose tissues. There is an about 65-70% homology between β1/β3- and β2-receptors.

The role of β-adrenergic receptors in the lung is discussed, for example, in Johnson, M., Am. J. Respir. Crit. Care Med. 158:S146-S153 (1998), review. β2-adenoreceptors are widely distributed, and occur not only in airway smooth muscle cells but also other cells in the lung, such as epithelial and endothelial cells, type II cells, and mast cells. Transgenic overexpression of β2-adrenergic receptors in airway epithelial cells has been reported to decrease bronchoconstriction (MsGraw et al., Am. J. Physiol. Lung Cell. Mol. Physiol. 279:L379-89 (2000)). Targeted transgenic expression of β2-adrenergic receptors to type II cells was shown to increase alveolar fluid clearance (McGraw et al., Am. J. Physiol. Lung Cell Mol. Physiol 281:L895-903 (2001)).

The role of β-adrenergic receptors in the heart has also been extensively studied. For details of the role of β-adrenergic receptors in the heart see, e.g. Ligget S.B., J. Clin. Invest. 107:947-8 (2001); Moniotte and Balligand, Cardiovasc. Drug. Rev. 2):19-26 (2002), Review; Xiao, R. P., Sci STKE Oct. 16:2001(104):RE15; and Port and Bristow, J. Mol. Cell. Cardiol. 33:887-905 (2001). Low- and high-level transgenic expression of β2-adrenergic receptors has been reported to differentially affect cardiac hypertrophy and function in Gαq-overexpressing mice.

β-Adrenergic agonists, such as procaterol, albuterol, salmeterol, and formoterol, have been demonstrated to be useful as bronchodilators in treating airway diseases. For example, patients with asthma are often administered an inhaled β2-adrenergic receptor agonist, such as albuterol, for the treatment of episodic bronchospasms. The binding of agonist promotes the interaction between the intracellular domains of βARs and the heterotrimeric G-protein Gs. This interaction, in turn, catalyzes the exchange of GTP for GDP in the Gα subunit thereby activating Gα. The activated Gα activates adenylyl cyclase, catalyzing the synthesis of cAMP from ATP. The cAMP activates protein kinase A (PKA), resulting in downstream phosphorylation events. In particular, cAMP induces airway relaxation through phosphorylation of muscle regulatory proteins and attenuation of cellular Ca⁺⁺ concentration. For further details of the βAR signaling pathways, and for the action mechanism of β-adrenergic agonists see, e.g. Cross et al., Circ. Res. 85:1077-1084 (1999), and Mills, S. E., J. Anim. Sci. 80(E. Suppl. 1):E30-E35 (2002).

β-Agonist inotropic agents, such as dobutamine, are now in use in the management of congestive heart failure (CHF), and similar heart diseases.

Unfortunately, long term use of β-adrenergic receptor agonists as bronchodilators often results in attenuated patient response. Agonist-induced loss of βAR sensitivity includes (1) loss of receptor function through uncoupling from the G protein signal transducer, which effect is typically rapidly reversible; (2) sequestration of receptors inside the cell upon longer agonist exposure; and (3) degradation of the βARs. In addition, in certain disease states, the steady state level of βARs may be altered by agonist-independent means as well, either by affecting βAR synthesis, or βAR degradation rates. The latter mechanism has been demonstrated to play a role in various heart conditions, such as congestive heart failure (CHF), and is likely to play a role in cystic fibrosis (CF) and chronic obstructive pulmonary disease (COPD) as well.

For further details see, e.g. Ligget and Lefkowitz, “Adrenergic receptor-coupled adenylyl cyclase systems: Regulation of receptor function by phosphorylation, sequestration, and down-regulation.” In: D. R. Sibley and M. D. Houslay (ed.) Regulation of Cellular Signal Transduction Pathways by Desensitization and Amplification. pp. 71-96, John Wiley and Sons, New York, 1994; S. E. Mills, 2002, supra, and Johnson, M., 1998, supra.

If the decline in βAR sensitivity is induced by an agonist of the receptor, it is possible to compensate, within certain limits, for the decline in βAR responsiveness by incrementally increasing the dose of the agonist. However, this approach is limited by the agonist's therapeutic index. Higher doses might prove toxic or have other side-effects. Similarly, there are no reliable means available at this time to counteract loss in βAR sensitivity that occurs as a result of an agonist-independent mechanism, such as in CHF, COPD, or CF patients. Accordingly, there is a great clinical need for a new approach that counteracts a pathologic change within the βAR pathway, such as a loss in βAR sensitivity, regardless the underlying mechanism. If the goal is to counteract agonist-induced loss in βAR sensitivity, such approach would enable long-term clinical use of β-adrenergic receptor agonists, without decline in their efficacy, and/or would otherwise improve the patient's overall condition.

### SUMMARY OF THE INVENTION

In one aspect, the invention concerns a method for counteracting a pathologic change within the β-adrenergic pathway in a mammalian subject by administering an effective amount of a compound capable of inhibiting TGF-β signaling through a TGF-β receptor.

In another aspect, the invention concerns a method for counteracting a loss in β-adrenergic receptor (βAR) sensitivity in a mammalian subject by administering an effective amount of a compound capable of inhibiting TGF-β signaling through a TGF-β receptor. In a particular embodiment, the loss in βAR sensitivity is induced by a βAR agonist. In another embodiment, TGF-β1 is used. In yet another embodiment, the βAR is β2AR.

In yet another aspect, the invention concerns a method for selective inhibition of β2-adrenergic receptor (β2-AR) expression and response to a β-adrenergic receptor antagonist, comprising treating a cell expression the β2-AR with a compound capable of TGF-β signaling through a TGF-β receptor.

### BRIEF DESCRIPTION OF THE DRAWINGS

FIG. 1 illustrates that TGFβ1 exposure reduces β2AR mRNA in human bronchial smooth muscle cells.

FIG. 2 shows that TGFβ1 exposure reduces βAR binding sites on hBSMC.

FIG. 3 shows the time course of TGFβ1 effect on procaterol-induced and forskolin-induced cAMP accumulation in hBSMC.

FIG. 4 shows that a representative small molecule TGFβ-RI kinase inhibitor (Compound No. 79) prevents TGFβ1-induced loss of adrenergic responsiveness in hBSMC.

FIG. 5 shows that p38 kinase is also involved in TGFβ1-regulated βAR signaling in hBSMC.

FIG. 6 shows that activin A, at higher concentration, causes loss of β2AR response, as well as reduced AC activity. These effects were reversible by a representative smal molecule TGFβ1 inhibitor of the present invention.

FIG. 7 shows that TGFβ1 downregulates β2AR mRNA in rat neonatal cardiomyocytes.

FIG. 8 shows that TGFβ1 induces Smad2 phosphorylation and causes loss of β2AR response in rat cardiomyocytes.

FIG. 9 shows that a representative small molecule compound of formula (1) (Compound No. 79) prevents TGFβ1-induced loss of β2AR response and AC activity in rat neonatal cardiomyocytes.

FIG. 10 Activin down-regulated β2AR mRNA in rat neonatal cardiomyocytes, and this down-regulation can be prevented by a representative small-molecule TGFβ1 inibitor (Compound No. 79).

FIG. 11 shows that activin A and IL-1β induce loss of β2AR response/AC activity in rat neonatal cardiomyocytes.

FIG. 12 shows that TGFβ1 induces Smad2 phosphorylation and down-regulates Smad3 expression in hBSMC.

FIG. 13 shows that a representative compound of formula (1) (Compound No. 79) blocks TGFβ1-induced Smad2 phosphorylation and Smad3 down-regulation in hBSMC.

FIG. 14 shows that TGFβ1 exposure induces Smad2/3 transient translocation into the nucleus in hBSMC.

FIG. 15 illustrates the TGF-β signal transduction pathway.

FIG. 16 illustrates the β-adrenergic receptor signal transduction pathway.

FIG. 17 illustrates the various mechanisms of β-adrenergic receptor degradation.

FIG. 18. β1- and β2-AR mediated cAMP accumulation in rat neonatal cardiomyocytes. Cardiomyocytes were pre-incubated with vehicle (no antagonist), ICI 118, 551 (β2-AR antagonist), CGP-20712A (β1-AR antagonist), or both antagonists in serum-free media containing the phosphodiesterase inhibitor IBMX (200 μM) for 30 min before being treated with 1 μM isoproterenol (Iso) for 10 min. Intracellular cAMP accumulation was measured by EIA, expressed as pmol/ml cell lysate as described in the Material and Methods.

FIG. 19. TGF-β1 induces reduction in β2-AR response. A, Concentration-dependent effects of TGF-β1 on procaterol stimulation of cAMP accumulation. Cardiomyocytes were incubated in the absence (control) or presence of various concentrations of TGF-β1 as indicated for 24 hr. Cells were washed and then cAMP accumulation stimulated by 10 μM procaterol was measured by EIA. *P<0.05 vs. control. B, Time-dependent effects of TGF-β1 on procaterol stimulated cAMP accumulation. Cardiomyocytes were incubated in absence or presence of 2 ng/ml TGF-β1 for indicated time. cAMP accumulation stimulated by 10 μM procaterol was measured by EIA. *P<0.05 vs. control. C, TGF-β1 effects on β1-AR and β2-AR mediated cAMP accumulation. Cardiomyocytes were pretreated with 1 ng/ml TGF-β1 for 24 hr, followed by incubation with 1 μM Iso for 10 min in the presence of ICI 118, 551 or CGP-20712A. cAMP accumulation was then measured. *P<0.05 vs. control. D, Effects of TGF-β1 on Iso- and forskolin-stimulated cAMP accumulation. Following incubation with TGF-β1 for 24 hr, cardiomyocytes were stimulated with control media (basal), 1 μM Iso, or 25 μM forskolin for 10 min in the presence of 200 μM IBMX. *P<0.05 vs. control.

FIG. 20. TGF-β1 exposure reduces the steady-state levels of β2-AR mRNA. Cardiomyocytes were treated either with various concentrations of TGF-β1 for 24 hr (A) or with 5 ng/ml of TGF-β1 for the indicated time periods (B) before harvested. Total RNA from each treatment was then extracted and subjected to real-time RT-PCR analyses of β1-AR and β2-AR message levels. 18S rRNA was used as an internal control.

FIG. 21. Modulation of β-adrenergic signaling molecules by TGF-β1. Real-time RT-PCR analyses for GRK2 (A), adenylyl cyclase AC5, (D) and AC6 (E) mRNA levels in TGF-β1 (5 ng/ml) treated cardiomyocytes at different time points as indicated. 18S rRNA was used as an internal control. AC5 and AC6 mRNA levels were significantly reduced. No change in GRK2 mRNA level was observed. Western blot analyses with specific antibodies for GRK2 (B) and G-proteins (stimulatory G protein, Gsα; inhibitory G proteins Giα-1 and Giα-3) (C) in untreated (control) or TGF-β1 (2 ng/ml) treated cardiomyocytes at 24 hr or at different time points as indicated. No change in GRK2 or G-protein levels was observed.

FIG. 22. Compound No. 79 (see Table 2) blocks TGF-β1-induced Smad2 activation and Smad2/3/4 nuclear translocation. A, Kinetics of Smad2 phosphorylation/activation induced by TGF-β1. Cardiomyocytes were treated with 2 ng/ml of TGF-β1 for various periods of time as indicated. Cell lysates were immunoblotted with antibodies against either phospho-specific Smad2 or total Smad2, respectively. B, Abrogation of TGF-β1-induced Smad2 activation by Compound No. 79. Cell lysates were immunoblotted with antibodies against phospho-specific Smad2, Actin and total Smad2, respectively, at 1 and 24 hr after incubation without or with 2 ng/ml of TGF-β1 in the absence (−) or presence (+) of 400 nM Compound No. 79 or a p38 inhibitor. Compounds were pre-incubated for 30 min before TGF-β1 was added. C, Inhibition of TGF-β1-induced Smad2/3 and Smad4 nuclear translocation by Compound No. 79. Cardiomyocytes were treated without or with 2 ng/ml of TGF-β1 for 60 min before being fixed for immunofluorescence staining using antibodies against Smad4 and Smad2/3, respectively. In the case of compound treatment, cells were pre-incubated with 400 nM Compound No. 79 for 30 min before TGF-β1 was added. DMSO was used as vehicle control.

FIG. 23. Compound No. 79 inhibits TGF-β1 induced down-regulation of gene expression. Cells were pre-incubated with various concentrations of Compound No. 79 or a p38 inhibitor before being treated with 5 ng/ml of TGF-β1 for 24 hr. Total RNA from each treatment was extracted and analysed by real-time RT-PCR for relative mRNA levels of Smad3 (A), β2-AR (B), AC5 (C) and AC6 (D). 18S rRNA was used as an internal control.

FIG. 24. TβRI inhibitor Compound No. 79, but not MAP kinase inhibitors, reverses TGF-β1-induced reduction of β2-adrenergic response as well as AC activity. A, Inhibitor effects on procaterol stimulated cAMP accumulation. Cardiomyocytes were treated with DMSO (vehicle), TGF-β monoclonal antibody (mAb), Compound No. 79 (200 nM), p38 inhibitor (0.5 μM), U-0126 (5 μM), or JNK inhibitor I (5 μM) in the absence or presence of TGF-β1 (1 ng/ml) for 24 hr. Intracellular cAMP accumulation stimulated by 10 μM procaterol was measured. *P<0.05 vs. control. **P<0.05 vs. DMSO. B, Inhibitor effects on forskolin stimulated cAMP accumulation. Cells were treated similarly as in A, and cAMP accumulation stimulated by 25 μM forskolin was measured. *P<0.05 vs. control. **P<0.05 vs. DMSO.

FIG. 25 illustrates the alteration of β-AR binding sites by TGF-β1 and Compound No. 79 in cardiomyocytes.

### DETAILED DESCRIPTION OF THE PREFERRED EMBODIMENT

A. Definitions

The terms “β-adrenergic receptor,” β-adrenoreceptor,” and “βAR” are used interchangeably, and encompass all groups of β-adrenergic receptors, including β1-, β2- and β-adrenergic receptors of all mammalian species, including human, as well as their polymorphic variants.

The term “TGF-β” is used herein to include native sequence TGF-β1, TGF-β2 and TGF-β3 of all mammalian species, including any naturally occurring variants of the TGF-β polypeptides.

The term “pathologic change in a β-adrenergic pathway” is used herein in the broadest sense and refers to any change in the mRNA or protein level, synthesis, density, activity, function, state of activation, or sensitivity of any member of a β-adrenergic receptor signal transduction pathway, including, without limitation, β1-, β2- and β-adrenergic receptors, cyclic adenosine monophosphate (cAMP), adenylyl cyclase, including the AC5 and AC6 isoforms, trimeric Gs protein, including α, β, and γ subunits, guanosine triphosphate (GTP), guanosine diphosphate (GDP), etc., that results in, or caused by, or associated with a disease or pathologic condition. For example, over- or under-expression, decreased sensitivity, reduced density of a β-adrenergic receptor may be associated with various diseases or pathologic conditions, and are considered a pathologic change in a β-adrenergic pathway.

The term “counteracting a pathologic change” is used in the broadest sense, and refers to any action that prevents, circumvents, reverses, compensates for, slows down, blocks, or limits the pathologic change, regardless the underlying mechanism.

The terms “loss in β-adrenergic sensitivity,” and “loss in β-adrenergic receptor sensitivity,” as well as their grammatical variants, are used interchangeably, and refer to the attenuation of biological response signaled through a β-adrenergic receptor, despite continued presence of the stimulus triggering such response.

The terms “counteracting loss in β-adrenergic sensitivity,” and “counteracting loss in β-adrenergic receptor sensitivity,” as well as their grammatical equivalents, are used interchangeably and in the broadest sense, and encompass any action that prevents, circumvents, reverses, compensates for, slows down, blocks, or limits the loss in the sensitivity of a β-adrenergic receptor to exposure to a molecule that signals through such receptor, i.e. an agonist of the receptor, regardless of the underlying cause or mechanism. The terms specifically cover, but are not limited to, β-adrenergic receptor desensitization, uncoupling, sequestration, and down-regulation.

The term “desensitization” is used in the broadest sense, and means reduced response to a given dose of agonist following prior exposure to an agonist.

The term “sequestration,” with reference to β-adrenergic receptors, is used in the broadest sense, and describes a process that results in a loss of ligand binding sites provided by cell-surface β-adrenergic receptors, following exposure to β-adrenergic receptor agonists, regardless of the underlying mechanism.

The term “agonist” of a β-adrenergic receptor, as used herein refers to any molecule that is capable of signaling through a β-adrenergic receptor, and includes any native ligand of such receptor, and other molecules that mimic a biological activity of a native ligand of the receptor. Agonists specifically include agonist antibodies to a β-adrenergic receptor, native ligands of a β-adrenergic receptor, including ligand fragments, and peptide and non-peptide small molecules.

The preferred “biological activity” mediated by a β-adrenergic receptor is any activity that results in the improvement of the lung, cardiac and/or renal function of a mammalian subject.

The terms “improvement of lung function,” and “improvement of pulmonary function” are used interchangeably, and refer to an improvement in any parameter suitable to measure lung performance. Thus, improvement of pulmonary function can be measured, for example, in murine bleomycin-induced lung injury models, such as the bleomycin rat lung injury model, which monitors improvements in respiratory rate and tidal volume. Parameters that are typically monitored in human patients as a measure of lung function include, but are not limited to, inspiratory and expiratory flow rates, lung volume (also referred to as lung capacity), and diffusing capacity for carbon monoxide, ability to forcibly exhale, respiratory rate, and the like. Methods of quantitatively determining pulmonary function in patients are well known in the art, and include timed measurement of inspiratory and expiratory maneuvers to measure specific parameters. For example, forced vital capacity (FVC) measures the total volume in liters exhaled by a patient forcefully from a deep initial inspiration. This parameter, when evaluated in conjunction with the forced expired volume in one second (FEV₁), allows bronchoconstriction to be quantitatively evaluated. In addition to measuring volumes of exhaled air as indices of pulmonary function, the flow in liters per minute measured over differing portions of the expiratory cycle can be useful in determining the status of a patient's pulmonary function. In particular, the peak expiratory flow, taken as the highest air flow rate in liters per minute during a forced maximal exhalation, is well correlated with overall pulmonary function in a patient with respiratory diseases. Methods and tools for measuring these and similar parameters are well known in the art, and routinely used in everyday clinical practice.

The term “tidal volume” refers to the volume of air inspired or expired with each normal breath.

The terms “improvement in cardiac function,” and “improvement in heart function” are used interchangeably, and refer to improvement in any parameter suitable to measure cardiac performance. Suitable parameters, without limitation, include arrhythmia, (peripheral) vasoconstriction, level of circulating catecholamines, degree of ionotropy, and the like.

The term “improvement in renal function” refers to improvement in any parameter suitable to measure renal performance, such as, for example, measuring the plasma-clearance of various substances, three-dimensional computerized tomography, radioactive evaluation of renal function, and the like.

The term “biological activity mediated by a TGF-β receptor” and similar terms are used to refer to any activity associated with the activation of a TGF-β receptor, and downstream intracellular signaling events.

A “biological activity mediated by the TGFβ-R1 kinase receptor,” or “biological activity mediated by a TGFβ-R1 receptor” can be any activity associated with the activation of TGFβ-R1 and downsteam intracellular signaling events, such as the phosphorylation of Smad2/Smad3, or any signaling effect occurring in the Smad-independent signaling arm of the TGFβ signal transduction cascad, including, for example, p38 and ras.

The term “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. Thus, in the context of improving lung function, cardiac function, or renal function, treatment includes prevention and treatment of a disease or condition negatively impacting lung function, cardiac function or renal function, or otherwise benefiting from the improvement of lung function, cardiac function, or renal function, relieving one or more symptoms of such disease, or prevention and treatment of complications resulting from such disease, and reduction in mortality. In the case of lung function, treatment may also result in the improvement of exercise tolerance of patients with compromised lung function.

The “pathology” of a disease or condition negatively impacting lung function includes all phenomena that compromise the well-being of the patient.

A “disease or condition benefiting from the improvement of lung function” includes all diseases, disorders and conditions which involve a negative change in at least one parameter suitable for measurement of lung performance. Such diseases and conditions include, without limitation, bronchoconstrictive diseases, and specifically, emphysema, chronic bronchitis, chronic obstructive pulmonary disease (COPD), pulmonary edema, cystic fibrosis (CF), occlusive lung disease, acute respiratory deficiency syndrome (ARDS), asthma, radiation-induced injury of the lung, and lung injuries resulting from other factors, such as, infectious causes, inhaled toxins, or circulating exogenous toxins, aging and genetic predisposition to impaired lung function.

A “disease or condition benefiting from the improvement of cardiac function” includes all diseases, disorders and conditions, which involve a negative change in at least one parameter suitable for measurement of cardiac performance. Such diseases and conditions include, without limitation, cardiac hypertrophy, congestive heart failure, cardiac myopathy, and the like.

A “disease or condition benefiting from the improvement of renal function” includes all diseases, disorders and conditions, which involve a negative change in at least one parameter suitable for measurement of renal performance. Such diseases and conditions include, without limitation, acute and chronic kidney diseases, renal failure and hemolytic uremic syndrome.

The term “TGF-β inhibitor” as used herein refers to a molecule having the ability to inhibit a biological function of a native TGF-β molecule mediated by a TGF-β receptor kinase, such as the TGFβ-R1 or TGFβ-R2 receptor, by interacting with a TGF-β receptor kinase. Accordingly, the term “inhibitor” is defined in the context of the biological role of TGF-β and its receptors. While the inhibitors herein are characterized by their ability to interact with a TGF-β receptor kinase and thereby inhibiting TGF-β biological function, they might additionally interact with other members in the TGF-β signal transduction pathway or members shared by the TGF-β signal transduction pathway and another pathway. Thus, the term “TGF-β inhibitor” specifically includes molecules capable of interacting with and inhibiting the biological function of two or more receptor kinases, including, without limitation, an activin receptor kinase, e.g. Alk4, and/or a MAP kinase.

The term “interact” with reference to an inhibitor and a receptor includes binding of the inhibitor to the receptor as well as indirect interaction, which does not involve binding. The binding to a receptor can, for example, be specific or preferential.

The terms “specifically binding,” “binds specifically,” “specific binding,” and grammatical variants thereof, are used to refer to binding to a unique epitope within a target molecule, such as a TGFβ receptor, e.g. the type I TGF-β receptor (TGFβ-R1). The binding must occur with an affinity to effectively inhibit TGF-β signaling through the receptor, e.g. TGFβ-R1.

The terms “preferentially binding,” binds preferentially,” “preferential binding,” and grammatical variants thereof, as used herein means that binding to one target is significantly greater than binding to any other binding partner. The binding affinity to the preferentially bound target is generally at least about two-fold, more preferably at least about five-fold, even more preferably at least about ten-fold greater than the binding affinity to any other binding partner.

The term “preferentially inhibit” as used herein means that the inhibitory effect on the target that is “preferentially inhibited” is significantly greater than on any other target. Thus, for example, in the context of preferential inhibition of TGF-β-R1 kinase relative to the p38 kinase, the term means that the inhibitor inhibits biological activities mediated by the TGF-β-R1 kinase significantly more than biological activities mediated by the p38 kinase. The difference in the degree of inhibition, in favor of the preferentially inhibited receptor, generally is at least about two-fold, more preferably at least about five-fold, even more preferably at least about ten-fold.

The term “mammal” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the mammal is human.

Administration “in combination with” one or more further therapeutic agents includes simultaneous (concurrent) and consecutive administration in any order.

A “therapeutically effective amount”, in the context of the present invention refers to an amount capable of counteracting a pathologic change in a β-adrenergic pathway, as defined above. In reference to the treatment of a disease or condition, the term “therapeutically effective amount” refers to an amount capable of invoking one or more of the following effects: (1) prevention of the disease or condition; (2) inhibition (i.e., reduction, slowing down or complete stopping) of the development or progression of the disease or condition; (3) inhibition (i.e., reduction, slowing down or complete stopping) of consequences of or complications resulting from such disease or condition; and (4) relief, to some extent, of one or more symptoms associated with such disease or condition, or symptoms of consequences of or complications resulting from such disease and/or condition.

As used herein, a “noninterfering substituent” is a substituent which leaves the ability of the compound of formula (1) to inhibit TGF-β activity qualitatively intact. Thus, the substituent may alter the degree of inhibition. However, as long as the compound of formula (1) retains the ability to inhibit TGF-β activity, the substituent will be classified as “noninterfering.”

As used herein, “hydrocarbyl residue” refers to a residue which contains only carbon and hydrogen. The residue may be aliphatic or aromatic, straight-chain, cyclic, branched, saturated or unsaturated. The hydrocarbyl residue, when indicated, may contain heteroatoms over and above the carbon and hydrogen members of the substituent residue. Thus, when specifically noted as containing such heteroatoms, the hydrocarbyl residue may also contain carbonyl groups, amino groups, hydroxyl groups and the like, or contain heteroatoms within the “backbone” of the hydrocarbyl residue.

As used herein, the term “alkyl,” “alkenyl” and “alkynyl” include straight- and branched-chain and cyclic monovalent substituents. Examples include methyl, ethyl, isobutyl, cyclohexyl, cyclopentylethyl, 2-propenyl, 3-butynyl, and the like. Typically, the alkyl, alkenyl and alkynyl substituents contain 1-10C (alkyl) or 2-10C (alkenyl or alkynyl). Preferably they contain 1-6C (alkyl) or 2-6C (alkenyl or alkynyl). Heteroalkyl, heteroalkenyl and heteroalkynyl are similarly defined but may contain 1-2 O, S or N heteroatoms or combinations thereof within the backbone residue.

As used herein, “acyl” encompasses the definitions of alkyl, alkenyl, alkynyl and the related hetero-forms which are coupled to an additional residue through a carbonyl group.

“Aromatic” moiety refers to a monocyclic or fused bicyclic moiety such as phenyl or naphthyl; “heteroaromatic” also refers to monocyclic or fused bicyclic ring systems containing one ore more heteroatoms selected from O, S and N. The inclusion of a heteroatom permits inclusion of 5-membered rings as well as 6-membered rings. Thus, typical aromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Any monocyclic or fused ring bicyclic system which has the characteristics of aromaticity in terms of electron distribution throughout the ring system is included in this definition. Typically, the ring systems contain 5-12 ring member atoms.

Similarly, “arylalkyl” and “heteroalkyl” refer to aromatic and heteroaromatic systems which are coupled to another residue through a carbon chain, including substituted or unsubstituted, saturated or unsaturated, carbon chains, typically of 1-6C. These carbon chains may also include a carbonyl group, thus making them able to provide substituents as an acyl moiety.

B. Modes of Carrying Out the Invention

The present invention is based on the surprising discovery that compounds capable of inhibiting TGFβ signaling through a TGFβ receptor can counteract pathologic changes in the β-adrenergic pathway. Accordingly, the invention concerns the administration to a mammalian, e.g. human, subject in need a compound capable of inhibiting TGFβ signaling through a TGFβ receptor.

As discussed above, a particular pathologic change in the β-adrenergic pathway is loss in β-adrenergic sensitivity, i.e. loss in the response of a β-adrenergic receptor to a stimulus. The loss in β-adrenergic sensitivity might result from a variety of reasons, including, but not limited to, long-term or excessive exposure to a β-adrenergic receptor agonist.

β-adrenergic receptor (βAR) agonists exert their biological activity by interacting with the ligand binding site of a β-adrenoreceptor. This interaction triggers a series of downstream events, including catalysis of the synthesis of cAMP from ATP by activated adenylyl cyclase. cAMP is known to induce airway relaxation through phosphorylation of muscle regulatory proteins, and attenuation of cellular Ca⁺⁺ concentration. Since βAR agonists induce the production of cAMP, they are potent smooth muscle relaxants.

The use of inhaled βAR agonists for bronchodilation is in wide-spread clinical use. There are numerous lung conditions, such as chronic obstructive pulmobary disease (COPD) benefit from treatment with β-receptor agonists. COPD is commonly used to describe a spectrum of conditions, diseases and symptoms that may occur individually or in combination, including, for example, chronic obstructive bronchitis, emphysema, and chronic airway obstruction. Over the time, as the diseases progress, gradually more serious symptoms can develop. Although COPD is a progressive disease, the severity of which increases over time, it is characterized by recurrent exacerbations of varying intensity, for example due to repeated exposure to environmental pollutants, cigarette smoke, and the like. COPD is currently the fourth leading cause of death in the United States. βAR agonists are also widely used in the treatment of other lung conditions that require or benefit from the improvement of lung function (in particular conditions that require of benefit from bronchodilation), including, without limitation, emphysema, chronic bronchitis, pulmonary edema, cystic fibrosis (CF), occlusive lung disease, acute respiratory deficiency syndrome (ARDS), asthma, radiation-induced injury of the lung, and lung injuries resulting from other factors, such as, infectious causes, inhaled toxins, or circulating exogenous toxins, aging and genetic predisposition to impaired lung function. In all instances, βAR agonists may be administered alone or in combination with other pharmacological agents, such as anticholinergic agents, theophylline, or corticosteroid therapy.

It is also well known that βAR-mediated cardiac inotropic responsiveness is critical to hemodynamic balance in the heart. In various forms of cardiac myopathy, cardiac hypertrophy and congestive heart failure (CHF) βAR pathways undergo several alterations that result in reduced adrenergic stimulation. Thus, hypertrophy and failure are characterized by marked abnormalities in βAR function (Bristow, Lancet 352 (Suppl. I) 8-14 (1998)). In the failing human heart, β1-AR is desensitized and selectively down-regulated, resulting in a weaker ionotropic response. β2-AR may be desensitized in the failing heart, but receptor levels are not significantly changed, resulting in a ratio of β1-AR/β2-AR reminiscent of that in the developing myocardium (Bristow et al., Circ. Res. 59:297-309 (1986); Brodde and Michel, Pharmacol. Rev. 51:651-690 (1999); Ligget, J. Clin. Invest. 107:947-948 (2001)). It has been presumed that the increased catecholamines observed in heart failure are responsible, at least in part, for both β-AR desensitization and down-regulation (Bristow, 1998, supra; Bristow et al., N. Engl. J. Med. 307:205-211 (1982)). However, agonist induced down-regulation does not explain subtype specific loss of β1-AR; thus, other mechanisms may be operative. Increasing evidence suggest that various growth factors such as transforming growth factor-β1 (TGF-β1), epidermal growth factor (EGF), and nerve growth factor (NGF) can modulate β-AR signaling in the heart of experimental models under pathological conditions (Nair et al., J. Cel. Physiol. 164:232-239 (1995); Lorita et al., Am. J. Physiol. Heart. Circ. Physiol. 283:H1887-1895 (2002); Heath et al., J. Physiol. 512:779-791 (1998)). The administration of exogenous β-agonist inotropic agents, such as dobutamine, benefits patients with advanced forms of these and similart heart conditions.

Commercially available β-adrenergic receptor agonists include albuterol (PROVENTIL®), which can be considered as a prototype of β2-agonists that selectively interact with the β2 receptor, fenoterol, formoterol, pirbuterol, procaterol, and dobutamine. β-receptor agonists are required to interact with the active site of a β-adrenergic receptor in order to exert their biological activities. Agonist binding/interaction sites of β-adrenergic receptors, such as the β2-adrenergic receptor, are well known, and the mechanism of interaction between the receptor and an agonist of the receptor is also well characterized (see, e.g. Strader et al., J. Biol. Chem. 264:13572-13580 (1989)).

Unfortunately, patients subject to long-term or excessive exposure to β-agonists are likely to develop tolerance to such treatment, typically as a result of receptor desensitization, uncoupling, sequestration and/or down-regulation. The risk is particularly high in the case of rapidly acting inhaled agents, such as albuterol, used as bronchodilators. Accordingly, these processes significantly limit the effectiveness of βAR agonists in the treatment of various lung conditions that benefit from bronchodilation. Similarly, failing hearts often exhibit depressed responsiveness to the administration of β-agonist inotropic agents. Thus, various forms of cardiomyopathy and CHF have been shown to involve down-regulation and/or uncoupling of β1ARs and uncoupling of β2ARs. For example, in cardiac fibroblasts, TGF-β1 has been shown to down-regulate β-AR number and response to isoproterenol (Iizuka et al., J. mol. Cel. Cardiol. 26:435-440 (1994)). Recently Rozankranz et al. reported that over-expression of circulating TGF-β1 in transgenic (TG) mice induced cardiac hypertrophy and enhanced β-adrenergic signaling (Am. J. Physiol. Heart. Circ. Physiol. 283:H1253-1262 (2002)). However, it is not clear whether the altered β-AR signaling in these mice reflects the direct effects of TGF-β1 or is due to secondary effects of cardiac hypertrophy caused by excess TGF-β1 in the TG system.

While much of the discussion so far has focused on agonist-induced loss in βAR responsiveness, in certain conditions, such as CF and COPD, βAR-sensitivity might decline in an agonist-independent manner as well. For example, in these and other disease states the steady state level of receptors may be altered either by decline in the synthesis of βAR as a result of the disease state, or as a result of an increase in the degradation rate of βAR.

The present invention provides a new and efficient way of improving impaired βAR responsiveness in mammalian subjects, such as humans. In a particular aspect, the present invention provides a new and efficient way of increasing patient responsiveness to β-agonist therapy by the administration of compounds capable of inhibiting TGF-β1 signaling through a TGFβ receptor.

Compounds of the Invention

The compounds of the present invention are capable of inhibiting TGFβ signaling through a TGFβ receptor and, as a result, can counteract pathologic changes in the β-adrenergic signal transduction pathway. As discussed earlier, a TGF-β inhibitor, as defined for the purpose of the present invention, can be any molecule having the ability to inhibit a biological function of a native TGF-β molecule mediated by a TGF-β receptor kinase, such as the TGFβ-R1 or TGFβ-R2 receptor via interaction with a TGF-β receptor kinase. Although the inhibitors are characterized by their ability to interact with a TGF-β receptor kinase and thereby inhibiting TGF-β biological function, they might additionally interact with other members in the TGF-β signal transduction pathway or members shared by the TGF-β signal transduction pathway and another pathway. Thus, TGF-β inhibitors might interact with two or more receptor kinases.

As discussed earlier, the type 1 and type 2 TGF-β receptors are serine-threonine kinases that signal through the Smad family of transcriptional regulators. Binding of TGF-β induces phosphorylation and activation of TGFβ-R1 by the TGFβ-R2. The activated TGFβ-R1 phosphorylates Smad2 and Smad3, which bind to Smad4 to move into the nucleus and form transcription regulatory complexes. Other signaling pathways, such as the MAP kinase-ERK cascade are also activated by TGF-β signaling, and modulate Smad activation. The Smad proteins couple the activation of both the TGF-β and the activin receptors to nuclear transcription. Thus, the TGF-β inhibitors of the present invention may additionally interact with an activin receptor kinase, such as Alk4, and/or a MAP kinase.

The compounds of the present invention include, without limitation, polypeptides, including antibodies and antibody-like molecules, peptides, polynucleotides, antisense molecules, decoys, and non-peptide small organic molecules that are capable of inhibiting TGF-β signaling through a TGF-β receptor.

In a particular embodiment, the compounds of the present invention are small organic molecules (non-peptide small molecules), generally less than about 1,000 daltons in size. Preferred non-peptide small molecules have molecular weights of less than about 750 daltons, more preferably less than about 500 daltons, and even more preferably less than about 300 daltons.

In a preferred embodiment, the compounds of the invention are of the formula

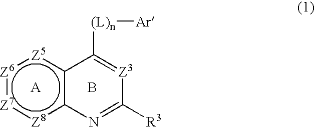

or the pharmaceutically acceptable salts thereof

wherein R³ is a noninterfering substituent;

each Z is CR² or N, wherein no more than two Z positions in ring A are N, and wherein two adjacent Z positions in ring A cannot be N;

each R² is independently a noninterfering substituent;

L is a linker;

n is 0 or 1; and

Ar′ is the residue of a cyclic aliphatic, cyclic heteroaliphatic, aromatic or heteroaromatic moiety optionally substituted with 1-3 noninterfering substituents.

In a preferred embodiment, the small organic molecules herein are derivatives of quinazoline and related compounds containing mandatory substituents at positions corresponding to the 2- and 4-positions of quinazoline. In general, a quinazoline nucleus is preferred, although alternatives within the scope of the invention are also illustrated below. Preferred embodiments for Z³ are N and CH; preferred embodiments for Z⁵-Z⁸ are CR². However, each of Z⁵-Z⁸ can also be N, with the proviso noted above. Thus, with respect to the basic quinazoline type ring system, preferred embodiments include quinazoline per se, and embodiments wherein all of Z⁵-Z⁸ as well as Z³ are either N or CH. Also preferred are those embodiments wherein Z³ is N, and either Z⁵ or Z⁸ or both Z⁵ and Z⁸ are N and Z⁶ and Z⁷ are CH or CR². Where R² is other than H, it is preferred that CR² occur at positions 6 and/or 7. Thus, by way of example, quinazoline derivatives within the scope of the invention include compounds comprising a quinazoline nucleus, having an aromatic ring attached in position 2 as a non-interfering substituent (R³), which may be further substituted.

With respect to the substituent at the positions corresponding to the 4-position of quinazoline, LAr′, L is present or absent and is a linker which spaces the substituent Ar′ from ring B at a distance of 2-8 Å, preferably 2-6 Å, more preferably 2-4 Å. The distance is measured from the ring carbon in ring B to which one valence of L is attached to the atom of the Ar′ cyclic moiety to which the other valence of the linker is attached. The Ar′ moiety may also be coupled directly to ring B (i.e., when n is 0). Typical, but nonlimiting, embodiments of L are of the formula S(CR²₂)ₘ, —NR¹SO₂(CR²₂)₁, NR¹(CR²₂)ₘ, NR¹CO(CR²₂)₁, O(CR²₂)ₘ, OCO(CR²₂)₁, and

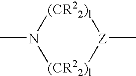

wherein Z is N or CH and wherein m is 0-4 and 1 is 0-3, preferably 1-3 and 1-2, respectively. L preferably provides —NR¹— coupled directly to ring B. A preferred embodiment of R¹ is H, but R¹ may also be acyl, alkyl, arylacyl or arylalkyl where the aryl moiety may be substituted by 1-3 groups such as alkyl, alkenyl, alkynyl, acyl, aryl, alkylaryl, aroyl, N-aryl, NH-alkylaryl, NH-aroyl, halo, OR, NR₂, SR, —SOR, —NRSOR, —NRSO₂R, —SO₂R, —OCOR, —NRCOR, —NRCONR₂, —NRCOOR, —OCONR₂, —RCO, —COOR, —SO₃R, —CONR₂, SO₂NR₂, CN, CF₃, and NO₂, wherein each R is independently H or alkyl (1-4C), preferably the substituents are alkyl (1-6C), OR, SR or NR₂ wherein R is H or lower alkyl (1-4C). More preferably, R¹ is H or alkyl (1-6C). Any aryl groups contained in the substituents may further be substituted by for example alkyl, alkenyl, alkynyl, halo, OR, NR₂, SR, —SOR, —SO₂R, —OCOR, —NRCOR, —NRCONR₂, —NRCOOR, —OCONR₂, —RCO, —COOR, SO₂R, NRSOR, NRSO₂R, —SO₃R, —CONR₂, SO₂NR₂, CN, CF₃, or NO₂, wherein each R is independently H or alkyl (1-4C).

Ar′ is aryl, heteroaryl, including 6-5 fused heteroaryl, cycloaliphatic or cycloheteroaliphatic. Preferably Ar′ is phenyl, 2-, 3- or 4-pyridyl, indolyl, 2- or 4-pyrimidyl, benzimidazolyl, indolyl, preferably each optionally substituted with a group selected from the group consisting of optionally substituted alkyl, alkenyl, alkynyl, aryl, N-aryl, NH-aroyl, halo, OR, NR₂, SR, —OOCR, —NROCR, RCO, —COOR, —CONR₂, SO₂NR₂, CN, CF₃, and NO₂, wherein each R is independently H or alkyl (1-4C).

Ar′ is more preferably indolyl, 6-pyrimidyl, 3- or 4-pyridyl, or optionally substituted phenyl.

For embodiments wherein Ar′ is optionally substituted phenyl, substituents include, without limitation, alkyl, alkenyl, alkynyl, aryl, alkylaryl, aroyl, N-aryl, NH-alkylaryl, NH-aroyl, halo, OR, NR₂, SR, —SOR, —SO₂R, —OCOR, —NRCOR, —NRCONR₂, —NRCOOR, —OCONR₂, RCO, —COOR, —SO₃R, —CONR₂, SO₂NR₂, CN, CF₃, and NO₂, wherein each R is independently H or alkyl (1-4C). Preferred substituents include halo, OR, SR, and NR₂ wherein R is H or methyl or ethyl. These substituents may occupy all five positions of the phenyl ring, preferably 1-2 positions, preferably one position. Embodiments of Ar′ include substituted or unsubstituted phenyl, 2-, 3-, or 4-pyridyl, 2-, 4- or 6-pyrimidyl, indolyl, isoquinolyl, quinolyl, benzimidazolyl, benzotriazolyl, benzothiazolyl, benzofuranyl, pyridyl, thienyl, furyl, pyrrolyl, thiazolyl, oxazolyl, imidazolyl, and morpholinyl. Particularly preferred as an embodiment of Ar′ is 3- or 4-pyridyl, especially 4-pyridyl in unsubstituted form.

Any of the aryl moieties, especially the phenyl moieties, may also comprise two substituents which, when taken together, form a 5-7 membered carbocyclic or heterocyclic aliphatic ring.

Thus, preferred embodiments of the substituents at the position of ring B corresponding to 4-position of the quinazoline include 2-(4-pyridyl)ethylamino; 4-pyridylamino; 3-pyridylamino; 2-pyridylamino; 4-indolylamino; 5-indolylamino; 3-methoxyanilinyl; 2-(2,5-difluorophenyl)ethylamino-, and the like.

R³ is generally a hydrocarbyl residue (1-20C) containing 0-5 heteroatoms selected from O, S and N. Preferably R³ is alkyl, aryl, arylalkyl, heteroalkyl, heteroaryl, or heteroarylalkyl, each unsubstituted or substituted with 1-3 substituents. The substituents are independently selected from a group that includes halo, OR, NR₂, SR, —SOR, —SO₂R, —OCOR, —NRCOR, —NRCONR₂, —NRCOOR, —OCONR₂, RCO, —COOR, —SO₃R, NRSOR, NRSO₂R, —CONR₂, SO₂NR₂, CN, CF₃, and NO₂, wherein each R is independently H or alkyl (1-4C) and with respect to any aryl or heteroaryl moiety, said group further including alkyl (1-6C) or alkenyl or alkynyl. Preferred embodiments of R³ (the substituent at position corresponding to the 2-position of the quinazoline) comprise a phenyl moiety optionally substituted with 1-2 substituents preferably halo, alkyl (1-6C), OR, NR₂, and SR wherein R is as defined above. Thus, preferred substituents at the 2-position of the quinazoline include phenyl, 2-halophenyl, e.g., 2-bromophenyl, 2-chlorophenyl, 2-fluorophenyl; 2-alkyl-phenyl, e.g., 2-methylphenyl, 2-ethylphenyl; 4-halophenyl, e.g., 4-bromophenyl, 4-chlorophenyl, 4-fluorophenyl; 5-halophenyl, e.g. 5-bromophenyl, 5-chlorophenyl, 5-fluorophenyl; 2,4- or 2,5-halophenyl, wherein the halo substituents at different positions may be identical or different, e.g. 2-fluoro-4-chlorophenyl; 2-bromo-4-chlorophenyl; 2-fluoro-5-chlorophenyl; 2-chloro-5-fluorophenyl, and the like. Other preferred embodiments of R³ comprise a cyclopentyl or cyclohexyl moiety.

As noted above, R² is a noninterfering substituent. As set forth above, a “noninterfering substituent” is one whose presence does not substantially destroy the TGF-β inhibiting ability of the compound of formula (1).

Each R² is also independently a hydrocarbyl residue (1-20C) containing 0-5 heteroatoms selected from O, S and N. Preferably, R² is independently H, alkyl, alkenyl, alkynyl, acyl or hetero-forms thereof or is aryl, arylalkyl, heteroalkyl, heteroaryl, or heteroarylalkyl, each unsubstituted or substituted with 1-3 substituents selected independently from the group consisting of alkyl, alkenyl, alkynyl, aryl, alkylaryl, aroyl, N-aryl, NH-alkylaryl, NH-aroyl, halo, OR, NR₂, SR, —SOR, —SO₂R, —OCOR, —NRCOR, —NRCONR₂, —NRCOOR, NRSOR, NRSO₂R, —OCONR₂, RCO, —COOR, —SO₃R, NRSOR, NRSO₂R, —CONR₂, SO₂NR₂, CN, CF₃, and NO₂, wherein each R is independently H or alkyl (1-4C). The aryl or aroyl groups on said substituents may be further substituted by, for example, alkyl, alkenyl, alkynyl, halo, OR, NR₂, SR, —SOR, —SO₂R, —OCOR, —NRCOR, —NRCONR₂, —NRCOOR, —OCONR₂, RCO, —COOR, —SO₃R, —CONR₂, SO₂NR₂, CN, CF₃, and NO₂, wherein each R is independently H or alkyl (1-4C). More preferably the substituents on R² are selected from R⁴, halo, OR⁴, NR⁴₂, SR⁴, —OOCR⁴, —NROCR⁴, —COOR⁴, R⁴CO, —CONR⁴₂, —SO₂NR⁴₂, CN, CF₃, and NO₂, wherein each R⁴ is independently H, or optionally substituted alkyl (1-6C), or optionally substituted arylalkyl (7-12C) and wherein two R⁴ or two substituents on said alkyl or arylalkyl taken together may form a fused aliphatic ring of 5-7 members.

R₂ may also, itself, be selected from the group consisting of halo, OR, NR₂, SR, —SOR, —SO₂R, —OCOR, —NRCOR, —NRCONR₂, —NRCOOR, NRSOR, NRSO₂R, —OCONR₂, RCO, —COOR, —SO₃R, NRSOR, NRSO₂R, —CONR₂, SO₂NR₂, CN, CF₃, and NO₂, wherein each R is independently H or alkyl (1-4C).

More preferred substituents represented by R² are those as set forth with regard to the phenyl moieties contained in Ar′ or R³ as set forth above. Two adjacent CR² taken together may form a carbocyclic or heterocyclic fused aliphatic ring of 5-7 atoms. Preferred R² substituents are of the formula R⁴, —OR⁴, SR⁴ or R⁴NH—, especially R⁴NH—, wherein R⁴ is defined as above. Particularly preferred are instances wherein R⁴ is substituted arylalkyl. Specific representatives of the compounds of formula (1) are shown in Tables 1-3 below. All compounds listed in Table 1 have a quinazoline ring system (Z³ is N), where the A ring is unsubstituted (Z⁵-Z⁸ represent CH). The substituents of the B ring are listed in the table.

TABLE 1

| Compound No. | L | Ar′ | R³ |
|---|---|---|---|
| 1 | NH | 4-pyridyl | 2-chlorophenyl |
| 2 | NH | 4-pyridyl | 2,6-dichlorophenyl |
| 3 | NH | 4-pyridyl | 2-methylphenyl |
| 4 | NH | 4-pyridyl | 2-bromophenyl |
| 5 | NH | 4-pyridyl | 2-fluorophenyl |
| 6 | NH | 4-pyridyl | 2,6-difluorophenyl |
| 7 | NH | 4-pyridyl | Phenyl |
| 8 | NH | 4-pyridyl | 4-fluorophenyl |
| 9 | NH | 4-pyridyl | 4-methoxyphenyl |
| 10 | NH | 4-pyridyl | 3-fluorophenyl |
| 11* | N* | 4-pyridyl | Phenyl |
| 12^{†} | N^{†} | 4-pyridyl | Phenyl |
| 13 | NHCH₂ | 4-pyridyl | Phenyl |
| 14 | NHCH2 | 4-pyridyi | 4-chlorophenyl |
| 15 | NH | 3-pyridyl | Phenyl |
| 16 | NHCH₂ | 2-pyridyl | Phenyl |
| 17 | NHCH₂ | 3-pyridyl | Phenyl |
| 18 | NHCH₂ | 2-pyridyl | Phenyl |
| 19 | NHCH₂CH₂ | 2-pyridyl | Phenyl |
| 20 | NH | 6-pyrimidinyl | Phenyl |
| 21 | NH | 2-pyrimidinyl | Phenyl |
| 22 | NH | phenyl | Phenyl |
| 23 | NHCH₂ | phenyl | 3-chlorophenyl |
| 24 | NH | 3-hydroxyphenyl | Phenyl |
| 25 | NH | 2-hydroxyphenyl | Phenyl |
| 26 | NH | 4-hydroxyphenyl | Phenyl |
| 27 | NH | 4-indolyl | Phenyl |
| 28 | NH | 5-indolyl | Phenyl |
| 29 | NH | 4-methoxyphenyl | Phenyl |
| 30 | NH | 3-methoxyphenyl | Phenyl |
| 31 | NH | 2-methoxyphenyl | Phenyl |
| 32 | NH | 4-(2- | Phenyl |
| | | hydroxyethyl)phenyl | |
| 33 | NH | 3-cyanophenyl | Phenyl |
| 34 | NHCH₂ | 2,5-difluorophenyl | Phenyl |
| 35 | NH | 4-(2-butyl)phenyl | Phenyl |
| 36 | NHCH₂ | 4-dimethylaminophenyl | Phenyl |
| 37 | NH | 4-pyridyl | Cyclopentyl |
| 38 | NH | 2-pyridyl | Phenyl |
| 39 | NHCH₂ | 3-pyridyl | Phenyl |
| 40 | NH | 4-pyrimidyl | Phenyl |
| 41^{‡} | N^{‡} | 4-pyridyl | Phenyl |
| 42 | NH | p-aminomethylphenyl | Phenyl |
| 43 | NHCH₂ | 4-aminophenyl | Phenyl |
| 44 | NH | 4-pyridyl | 3-chlorophenyl |
| 45 | NH | phenyl | 4-pyridyl |
| 46 | NH | 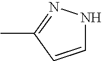 | Phenyl |
| 47 | NH | 4-pyridyl | t-butyl |
| 48 | NH | 2-benzylamino-3- | Phenyl |
| | | pyridyl | |
| 49 | NH | 2-benzylamino-4- | Phenyl |
| | | pyridyl | |
| 50 | NH | 3-benzyloxyphenyl | Phenyl |
| 51 | NH | 4-pyridyl | 3-aminophenyl |
| 52 | NH | 4-pyridyl | 4-pyridyl |
| 53 | NH | 4-pyridyl | 2-naphthyl |
| 54 | 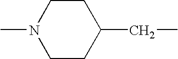 | 4-pyridyl | Phenyl |
| 55 | 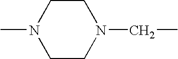 | phenyl | Phenyl |
| 56 | 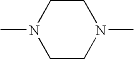 | 2-pyridyl | Phenyl |
| 57 | NHCH₂CH₂ | 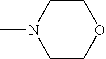 | Phenyl |
| 58 | not present | 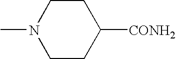 | Phenyl |
| 59 | not present | 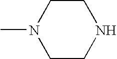 | Phenyl |
| 60 | NH | 4-pyridyl | Cyclopropyl |
| 61 | NH | 4-pyridyl | 2-trifluoromethyl |
| | | | phenyl |
| 62 | NH | 4-aminophenyl | Phenyl |
| 63 | NH | 4-pyridyl | Cyclohexyl |
| 64 | NH | 3-methoxyphenyl | 2-fluorophenyl |
| 65 | NH | 4-methoxyphenyl | 2-fluorophenyl |
| 66 | NH | 4-pyrimidinyl | 2-fluorophenyl |
| 67 | NH | 3-amino-4-pyridyl | Phenyl |
| 68 | NH | 4-pyridyl | 2- |
| | | | benzylaminophenyl |
| 69 | NH | 2-benzylaminophenyl | Phenyl |
| 70 | NH | 2-benzylaminophenyl | 4-cyanophenyl |
| 71 | NH | 3′-cyano-2- | Phenyl |
| | | benzylaminophenyl | |

*R¹ = 2-propyl
^{†}R¹ = 4-methoxyphenyl
^{‡}R¹ = 4-methoxybenzyl

The compounds in Table 2 contain modifications of the quinazoline nucleus as shown. All of the compounds in Table 2 are embodiments of formula (1) wherein Z³ is N and Z⁶ and Z⁷ represent CH. In all cases the linker, L, is present and is NH.

TABLE 2

| Compound | | | | |
|---|---|---|---|---|
| No. | Z⁵ | Z⁸ | Ar′ | R³ |
| 72 | CH | N | 4-pyridyl | 2-fluorophenyl |
| 73 | CH | N | 4-pyridyl | 2-chlorophenyl |
| 74 | CH | N | 4-pyridyl | 5-chloro-2- |
| | | | | fluorphenyl |
| 75 | CH | N | 4-(3-methyl)- | 5-chloro-2- |
| | | | pyridyl | fluorphenyl |
| 76 | CH | N | 4-pyridyl | Phenyl |
| 77 | N | N | 4-pyridyl | phenyl |
| 78 | N | CH | 4-pyridyl | Phenyl |
| 79 | N | N | 4-pyridyl | 5-chloro-2- |
| | | | | fluorphenyl |
| 80 | N | N | 4-(3-methyl)- | 5-chloro-2- |
| | | | pyridyl | fluorphenyl |

Additional compounds were prepared wherein ring A contains CR² at Z⁶ or Z⁷ where R² is not H. These compounds, which are all quinazoline derivatives, wherein L is NH and Ar′ is 4-pyridyl, are shown in Table 3.

TABLE 3

| Compound | | |
|---|---|---|
| No. | R³ | CR² as noted |
| 81 | 2-chlorophenyl | 6,7-dimethoxy |
| 82 | 2-fluorophenyl | 6-nitro |
| 83 | 2-fluorophenyl | 6-amino |
| 84 | 2-fluorophenyl | 7-amino |
| 85 | 2-fluorophenyl | 6-(3-methoxybenzylamino) |
| 86 | 2-fluorophenyl | 6-(4-methoxybenzylamino) |
| 87 | 2-fluorophenyl | 6-(2-isobutylamino) |
| 88 | 2-fluorophenyl | 6-(4- |
| | | methylmercaptobenzylamino) |
| 89 | 2-fluorophenyl | 6-(4-methoxybenzoyl amino) |
| 90 | 4-fluorophenyl | 7-amino |
| 91 | 4-fluorophenyl | 7-(3-methoxybenzylamino) |

Although the invention is illustrated with reference to certain quinazoline derivatives, it is not so limited. Inhibitors of the present invention include compounds having a non-quinazoline, such as, a pyridine, pyrimidine nucleus carrying substituents like those discussed above with respect to the quinazoline derivatives.

The compounds of the invention, including compounds of the formula (1) may be supplied in the form of their pharmaceutically acceptable acid-addition salts including salts of inorganic acids such as hydrochloric, sulfuric, hydrobromic, or phosphoric acid or salts of organic acids such as acetic, tartaric, succinic, benzoic, salicylic, and the like. If a carboxyl moiety is present on the compound of formula (1), the compound may also be supplied as a salt with a pharmaceutically acceptable cation.

Another group of compounds for use in the methods of the present invention is represented by the following formula (2)

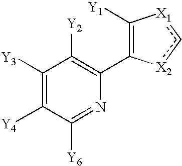

wherein Y₁ is phenyl or naphthyl optionally substituted with one or more substituents selected from halo, alkoxy(1-6C), alkylthio(1-6C), alkyl(1-6C), haloalkyl (1-6C), —O—(CH₂)ₘ-Ph, —S—(CH₂)ₘ-Ph, cyano, phenyl, and CO₂R, wherein R is hydrogen or alkyl(1-6C), and m is 0-3; or phenyl fused with a 5- or 7-membered aromatic or non-aromatic ring wherein said ring contains up to three heteroatoms, independently selected from N, O, and S:

Y₂, Y₃, Y₄, and Y₅ independently represent hydrogen, alkyl(1-6C), alkoxy(1-6C), haloalkyl(1-6C), halo, NH₂, NH-alkyl(1-6C), or NH(CH₂)ₙ-Ph wherein n is 0-3; or an adjacent pair of Y₂, Y₃, Y₄, and Y₅ form a fused 6-membered aromatic ring optionally containing up to 2 nitrogen atoms, said ring being optionally substituted by one o more substituents independently selected from alkyl(1-6C), alkoxy(a-6C), haloalkyl(1-6C), halo, NH₂, NH-alkyl(1-6C), or NH(CH₂)ₙ-Ph, wherein n is 0-3, and the remainder of Y₂, Y₃, Y₄, and Y₅ represent hydrogen, alkyl(1-6C), alkoxy(1-6C), haloalkyl(1-6C), halo, NH₂, NH-alkyl(1-6C), or NH(CH₂)ₙ-Ph wherein n is 0-3; and

one of X₁ and X₂ is N and the other is NR₆, wherein R₆ is hydrogen or alkyl(1-6C).

As used in formula (2), the double bonds indicated by the dotted lined represent possible tautomeric ring forms of the compounds. Further information about compounds of formula (2) and their preparation is disclosed in WO 02/40468, published May 23, 2002, the entire disclosure of which is hereby expressly incorporated by reference.

Yet another group of compounds for use in the methods of the invention is represented by the following formula (3)

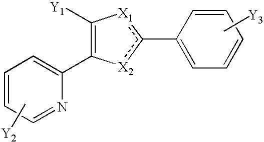

wherein Y₁ is naphthyl, anthracenyl, or phenyl optionally substituted with one or more substituents selected from the group consisting of halo, alkoxy(1-6C), alkylthio(1-6C), alkyl(1-6C), —O—(CH₂)-Ph, —S—(CH₂)ₙ-Ph, cyano, phenyl, and CO₂R, wherein R is hydrogen or alkyl(1-6C), and n is 0, 1, 2, or 3; or Y₁ represents phenyl fused with an aromatic or non-aromatic cyclic ring of 5-7 members wherein said cyclic ring optionally contains up to two heteroatoms, independently selected from N, O, and S;

Y₂ is H, NH(CH₂)ₙ-Ph or NH-alkyl(1-6C), wherein n is 0, 1, 2, or 3;

Y₃ is CO₂H, CONH₂, CN, NO₂, alkylthio(1-6C), —SO₂-alkyl(C1-6), alkoxy(C1-6), SONH₂, CONHOH, NH₂, CHO, CH₂NH₂, or CO₂R, wherein R is hydrogen or alkyl(1-6C);

one of X₁ and X₂ is N or CR′, and other is NR′ or CHR′ wherein R′ is hydrogen, OH, alkyl(C-16), or cycloalkyl(C3-7); or when one of X₁ and X₂ is N or CR′ then the other may be S or O.

Further details of the compounds of formula (3) and their modes of preparation are disclosed in WO 00/61576 published Oct. 19, 2000, the entire disclosure of which is hereby expressly incorporated by reference.

In a further embodiment, the TGF-β inhibitors of the present invention are represented by the following formula (4)

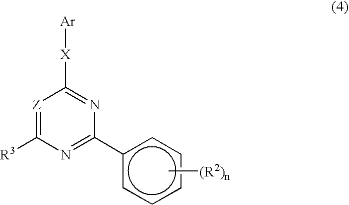

and the pharmaceutically acceptable salts and prodrug forms thereof; wherein

Ar represents an optionally substituted aromatic or optionally substituted heteroaromatic moiety containing 5-12 ring members wherein said heteroaromatic moiety contains one or more O, S, and/or N with a proviso that the optionally substituted Ar is not

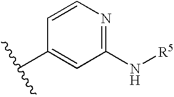

wherein R⁵ is H, alkyl (1-6C), alkenyl (2-6C), alkynyl (2-6C), an aromatic or heteroaromatic moiety containing 5-11 ring members;

X is NR¹, O, or S;

R¹ is H, alkyl (1-8C), alkenyl (2-8C), or alkynyl (2-8C);

Z represents N or CR⁴;

each of R³ and R⁴ is independently H, or a non-interfering substituent;

each R² is independently a non-interfering substituent; and

n is 0, 1, 2, 3, 4, or 5. In one embodiment, if n>2, and the R²'s are adjacent, they can be joined together to form a 5 to 7 membered non-aromatic, heteroaromatic, or aromatic ring containing 1 to 3 heteroatoms where each heteroatom can independently be O, N, or S.

In preferred embodiments, Ar represents an optionally substituted aromatic or optionally substituted heteroaromatic moiety containing 5-9 ring members wherein said heteroaromatic moiety contains one or more N; or

R¹ is H, alkyl (1-8C), alkenyl (2-8C), or alkynyl (2-8C); or

Z represents N or CR⁴; wherein

R⁴ is H, alkyl (1-10C), alkenyl (2-10C), or alkynyl (2-10C), acyl (1-10C), aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —SO₂R, —OCOR, —NRCOR, —NRCONR₂, —NRCOOR, —OCONR₂, —COOR, —SO₃R, —CONR₂, —SO₂NR₂, —CN, —CF₃, or —NO₂, wherein each R is independently H or alkyl (1-10C) or a halo or heteroatom-containing form of. said alkyl, each of which may optionally be substituted. Preferably R⁴ is H, alkyl (1-10C), OR, SR or NR₂ wherein R is H or alkyl (1-10C) or is O-aryl; or

R³ is defined in the same manner as R⁴ and preferred forms are similar, but R³ is independently embodied; or

each R² is independently alkyl (1-8C), alkenyl (2-8C), alkynyl (2-8C), acyl (1-8C), aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —NRSO₂R₂, —SO₂R, —OCOR, —OSO₃R, —NRCOR, —NRCONR₂, —NRCOOR, —OCONR₂, —COOR, —SO₃R, —CONR₂, SO₂NR₂, —CN, —CF₃, or —NO₂, wherein each R is independently H or lower alkyl (1-4C). Preferably R² is halo, alkyl (1-6C), OR, SR or NR₂ wherein R is H or lower alkyl (1-4C), more preferably halo; or n is 0-3.

The optional substituents on the aromatic or heteroaromatic moiety represented by Ar include alkyl (1-10C), alkenyl (2-10C), alkynyl (2-10C), acyl (1-10C), aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —SO₂R, —OCOR, —NRCOR, —NRCONR₂, —NRCOOR, —OCONR₂, —COOR, —SO₃R, —CONR₂, —SO₂NR₂, —CN, —CF₃, and/or NO₂, wherein each R is independently H or lower alkyl (1-4C). Preferred substituents include alkyl, OR, NR₂, O-alkylaryl and NH-alkylaryl.

In general, any alkyl, alkenyl, alkynyl, acyl, or aryl group contained in a substituent may itself optionally be substituted by additional substituents. The nature of these substituents is similar to those recited with regard to the primary substituents themselves.

Representative compounds of formula (4) are listed in the following Table 4.

| COM- | |
|---|---|
| POUND # | STRUCTURE |
| 92 | 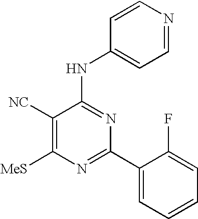 |
| 93 | 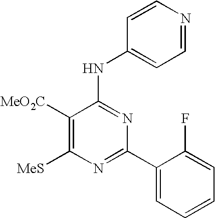 |
| 94 | 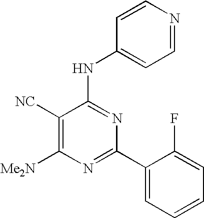 |
| 95 | 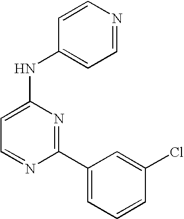 |
| 96 | 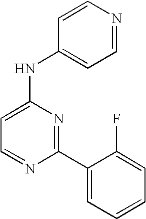 |
| 97 | 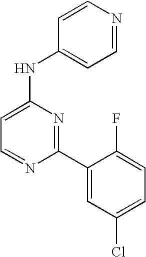 |
| 98 | 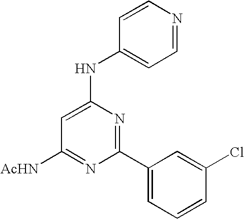 |
| 99 | 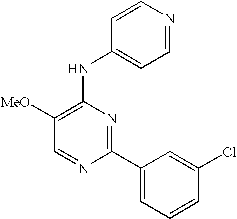 |
| 100 | 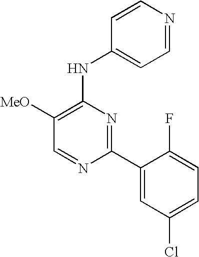 |
| 101 | 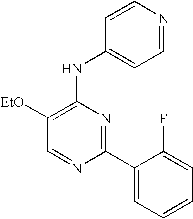 |
| 102 | 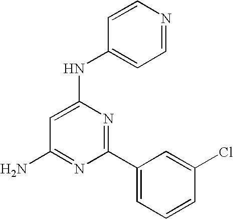 |
| 103 | 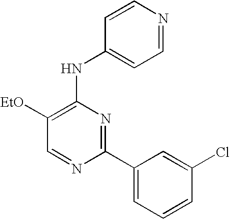 |
| 104 | 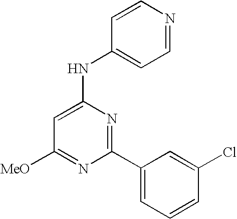 |
| 105 | 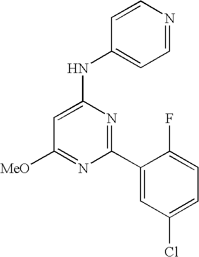 |
| 106 | 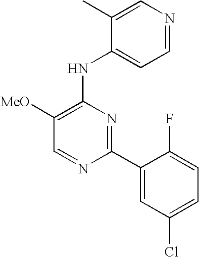 |
| 107 | 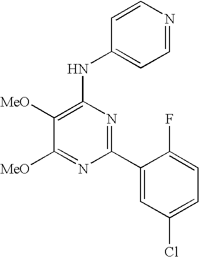 |
| 108 | 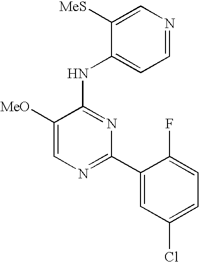 |
| 109 | 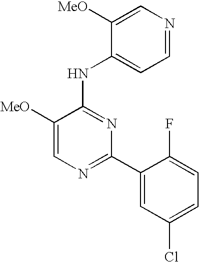 |
| 110 | 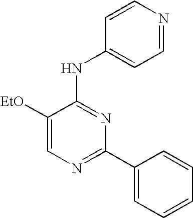 |
| 111 | 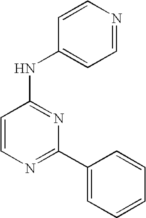 |
| 112 | 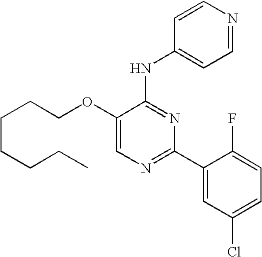 |
| 113 | 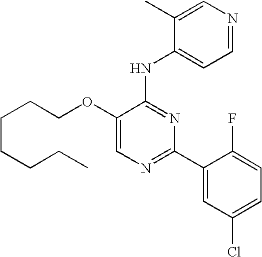 |
| 114 | 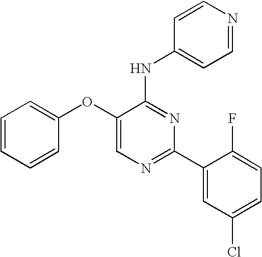 |
| 115 | 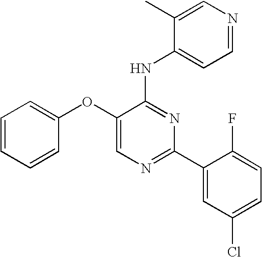 |
| 116 | 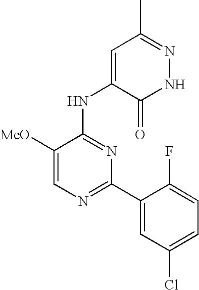 |
| 117 | 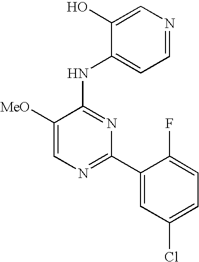 |
| 118 | 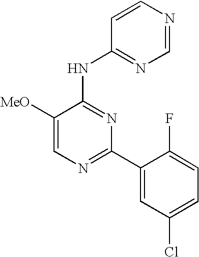 |
| 119 | 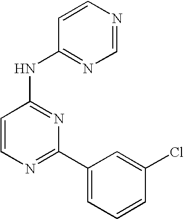 |
| 120 | 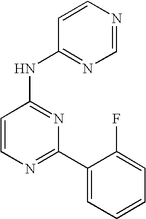 |
| 121 | 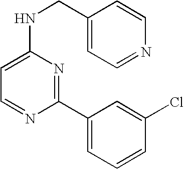 |
| 122 | 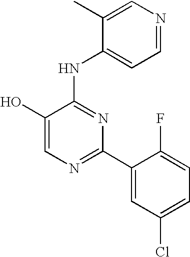 |
| 123 | 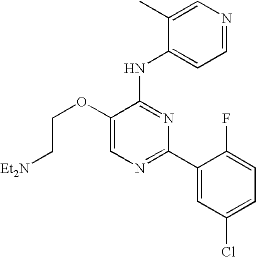 |
| 124 | 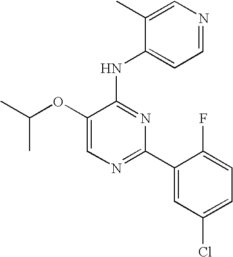 |
| 125 | 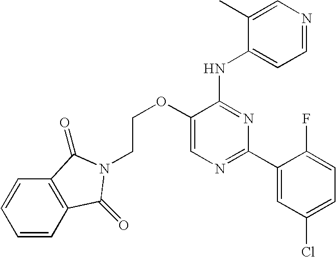 |
| 126 | 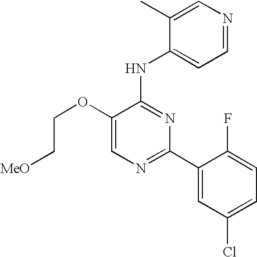 |
| 127 | 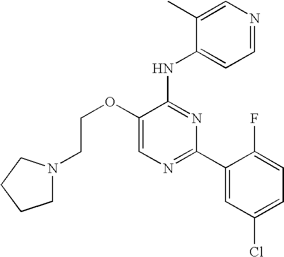 |
| 128 | 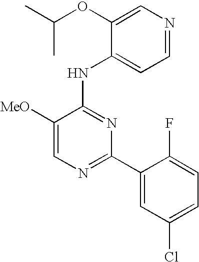 |
| 129 | 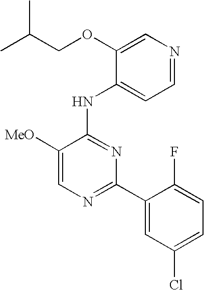 |
| 130 | 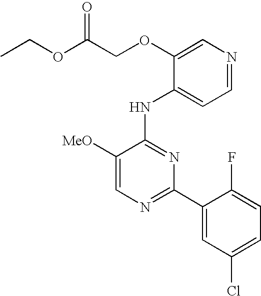 |
| 131 | 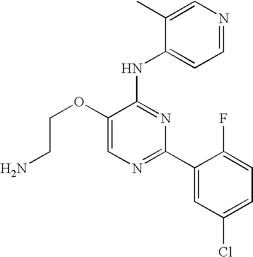 |
| 132 | 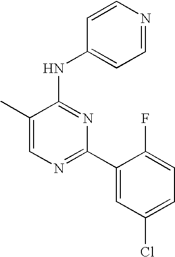 |
| 133 | 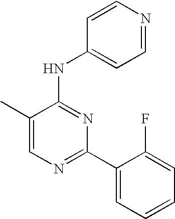 |
| 134 | 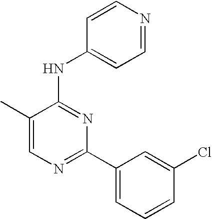 |
| 135 | 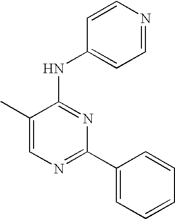 |
| 136 | 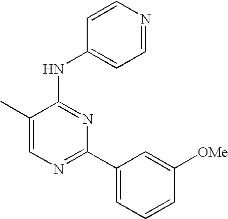 |
| 137 | 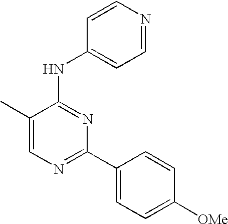 |
| 138 | 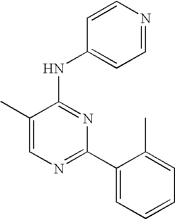 |
| 139 | 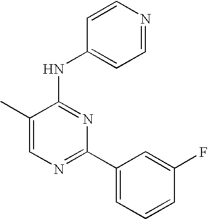 |
| 140 | 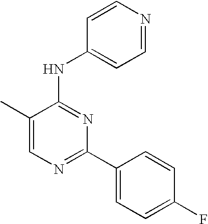 |
| 141 | 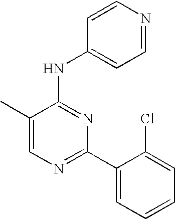 |
| 142 | 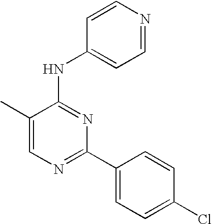 |
| 143 | 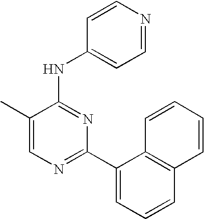 |
| 144 | 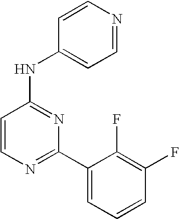 |
| 145 | 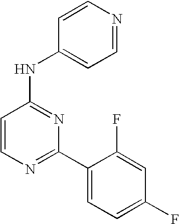 |
| 146 | 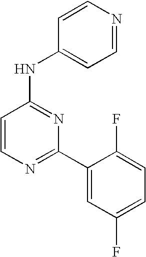 |
| 147 | 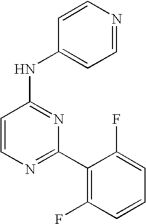 |
| 148 | 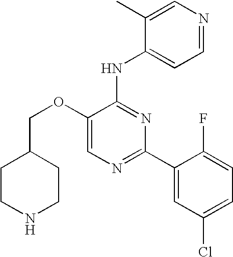 |
| 149 | 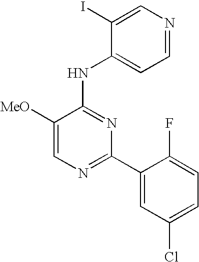 |
| 150 | 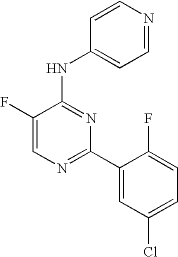 |
| 151 | 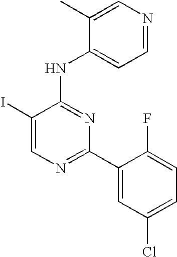 |
| 152 | 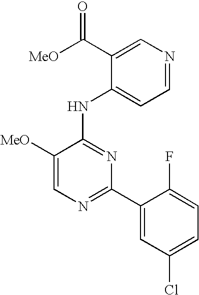 |
| 153 | 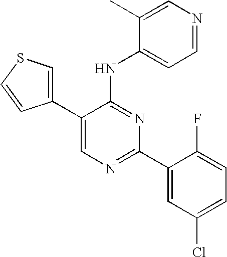 |
| 154 | 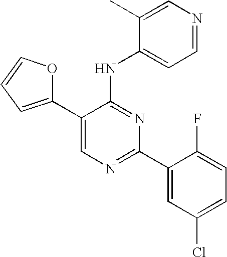 |
| 155 | 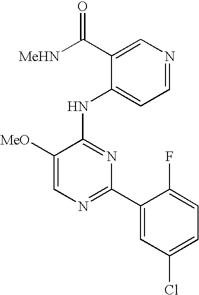 |
| 156 | 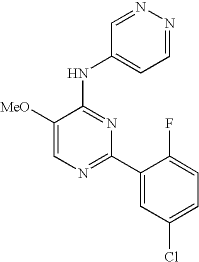 |
| 157 | 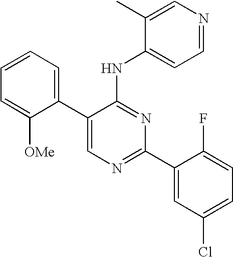 |
| 158 | 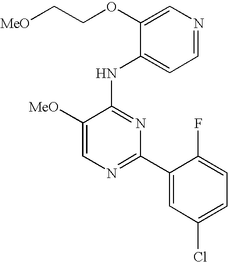 |
| 159 | 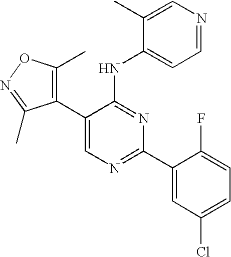 |
| 160 | 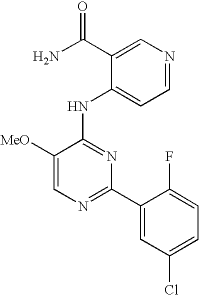 |
| 161 | 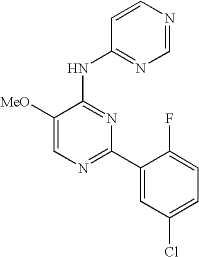 |
| 162 | 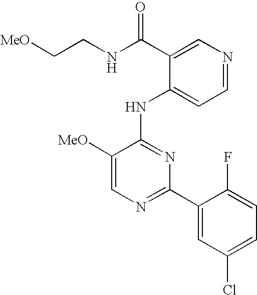 |
| 163 | 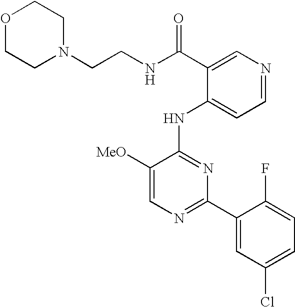 |
| 164 | 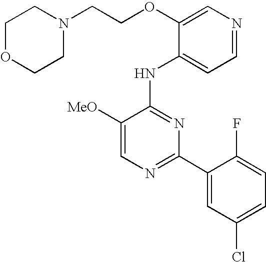 |
| 165 | 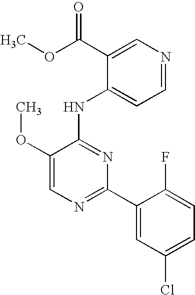 |
| 166 | 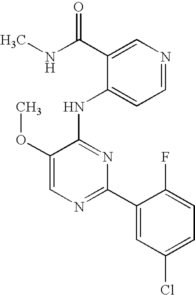 |
| 167 | 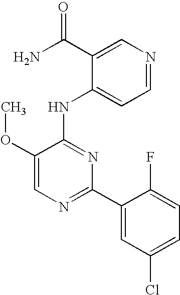 |
| 168 | 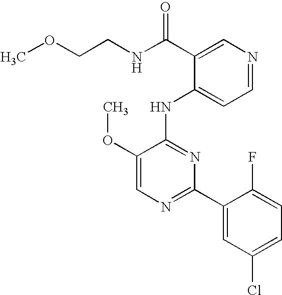 |
| 169 | 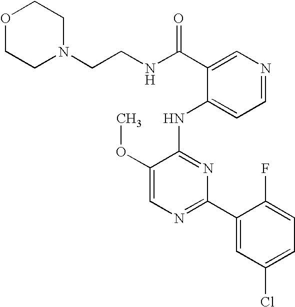 |
| 170 | 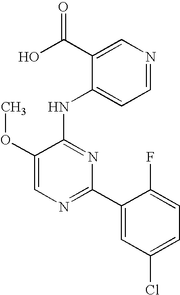 |
| 171 | 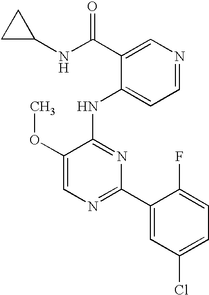 |
| 172 | 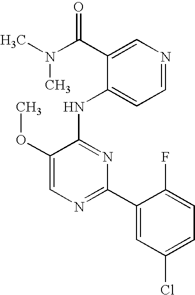 |
| 173 | 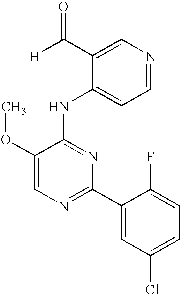 |
| 174 | 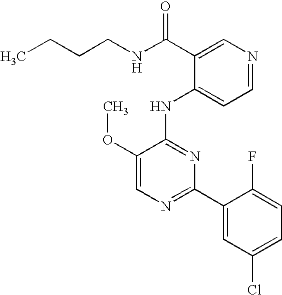 |
| 175 | 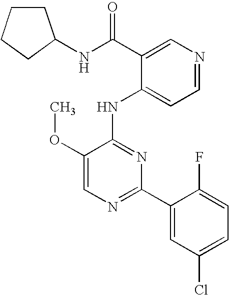 |
| 176 | 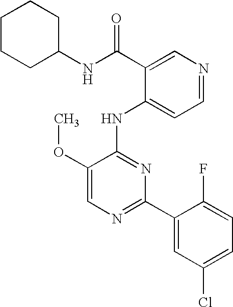 |
| 177 | 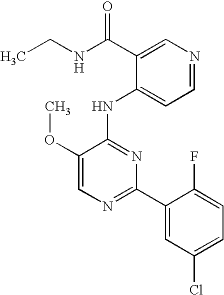 |
| 178 | 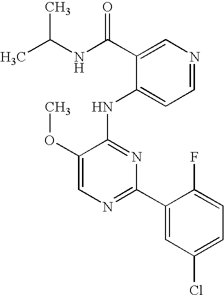 |
| 179 | 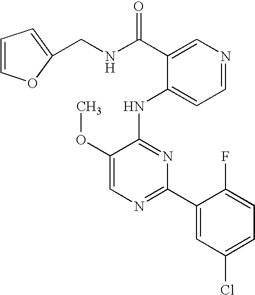 |
| 180 | 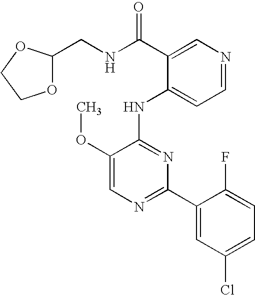 |
| 181 | 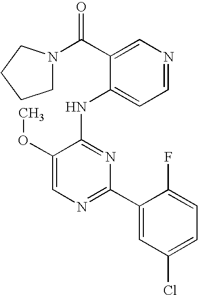 |
| 182 | 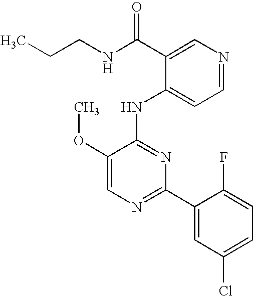 |
| 183 | 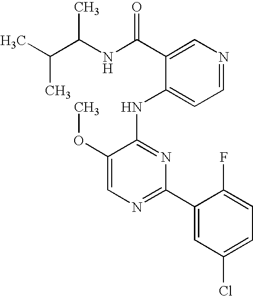 |
| 184 | 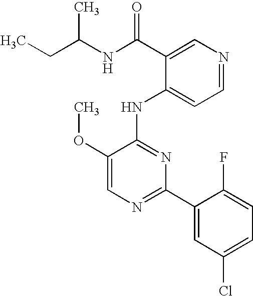 |
| 185 | 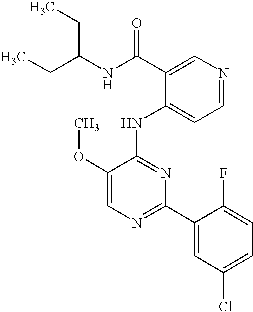 |
| 186 | 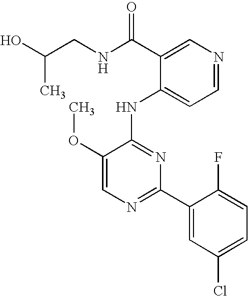 |
| 187 | 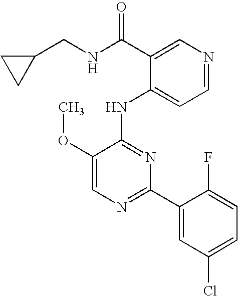 |
| 188 | 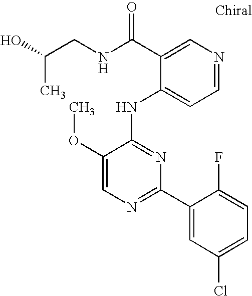 |
| 189 | 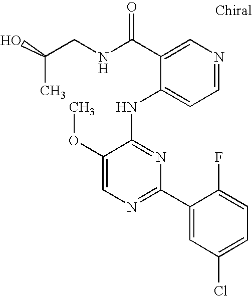 |
| 190 | 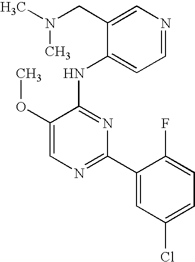 |
| 191 | 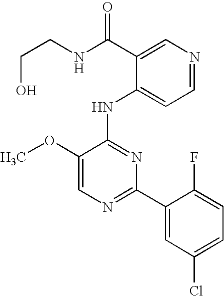 |
| 192 | 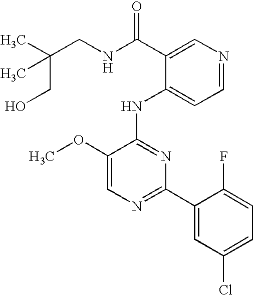 |
| 193 | 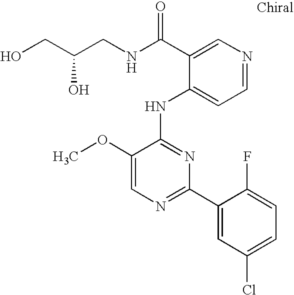 |
| 194 | 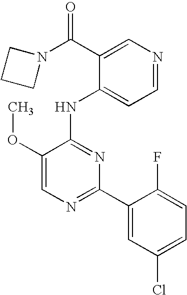 |
| 195 | 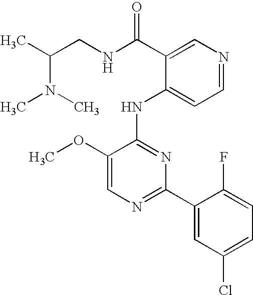 |
| 196 | 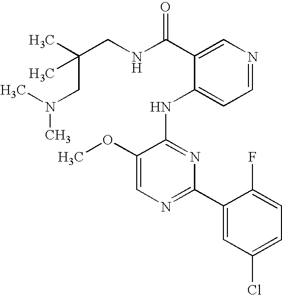 |
| 197 | 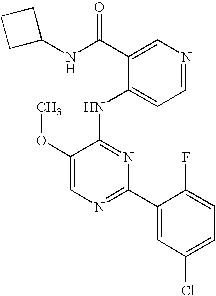 |
| 198 | 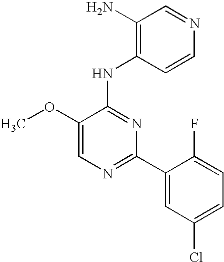 |
| 199 | 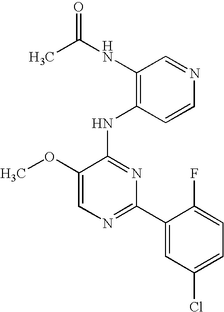 |
| 200 | 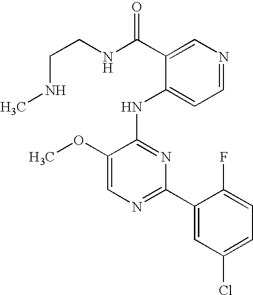 |
| 201 | 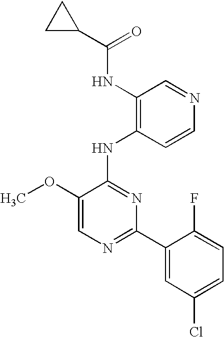 |
| 202 | 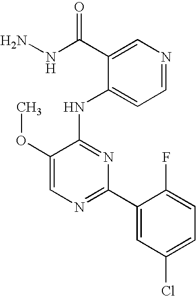 |
| 203 | 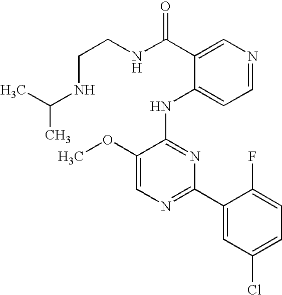 |
| 204 | 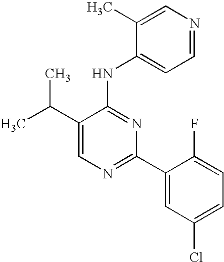 |
| 205 | 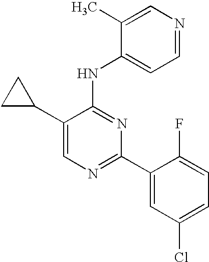 |
| 206 | 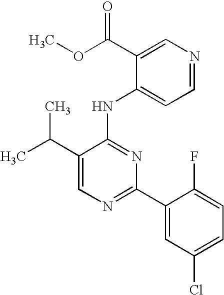 |
| 207 | 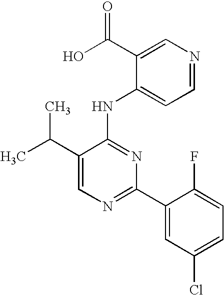 |
| 208 | 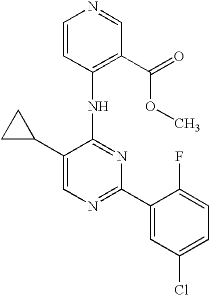 |
| 209 | 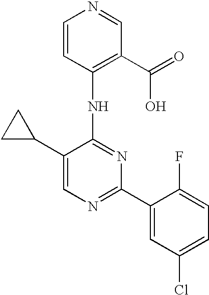 |
| 210 | 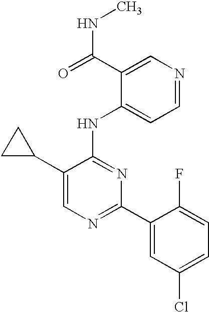 |
| 211 | 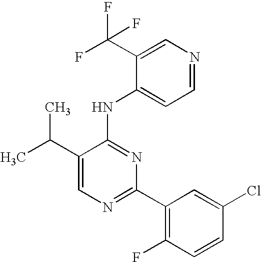 |
| 212 | 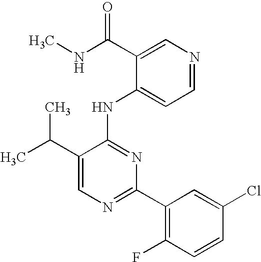 |
| 213 | 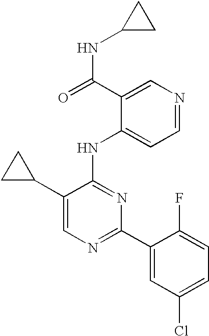 |
| 214 | 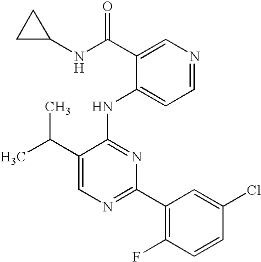 |
| 215 | 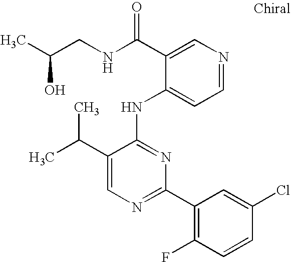 |
| 216 | 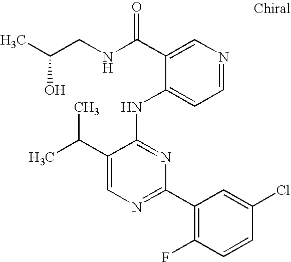 |
| 217 | 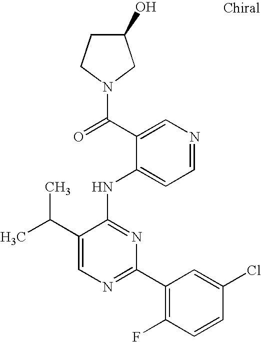 |
| 218 | 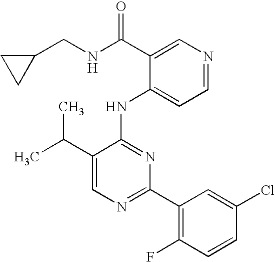 |
| 219 | 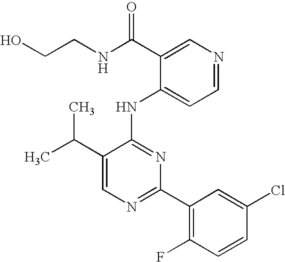 |
| 220 | 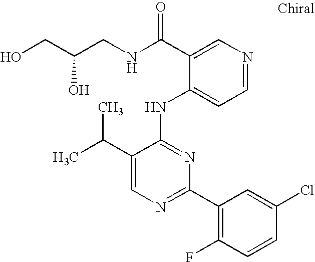 |
| 221 | 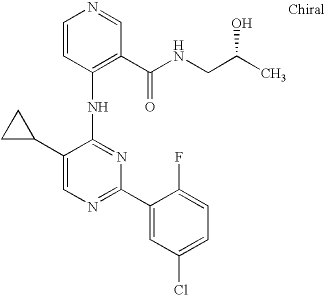 |
| 222 | 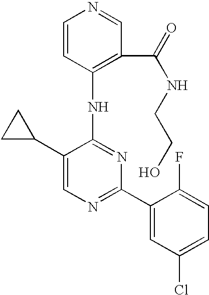 |
| 223 | 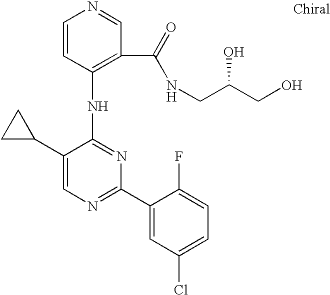 |
| 224 | 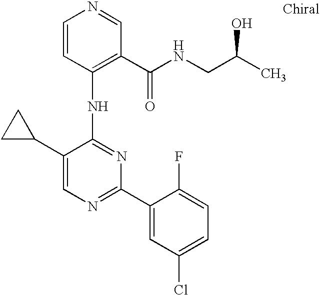 |
| 225 | 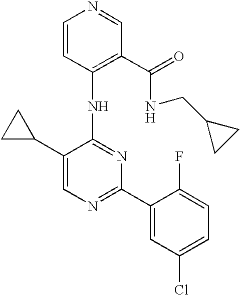 |
| 226 | 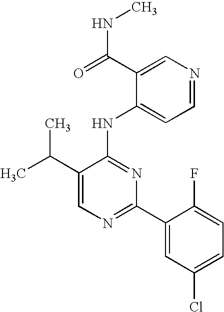 |
| 227 | 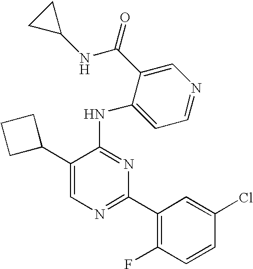 |
| 228 | 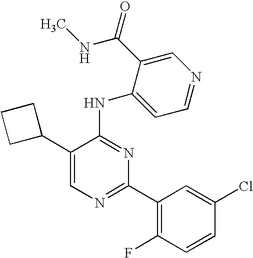 |
| 229 | 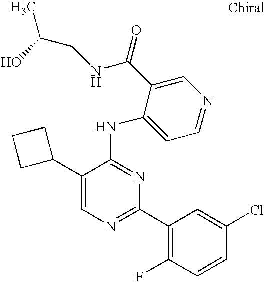 |
| 230 | 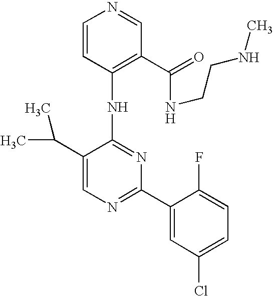 |
| 231 | 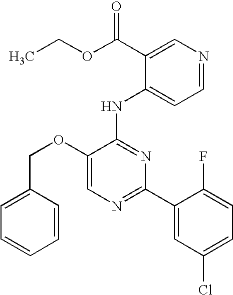 |
| 232 | 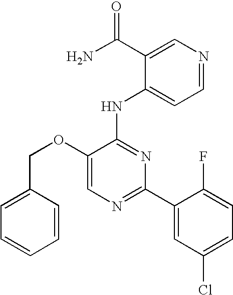 |
| 233 | 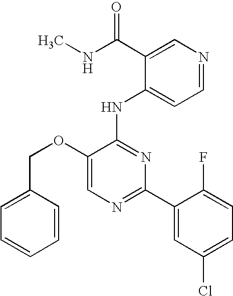 |
| 234 | 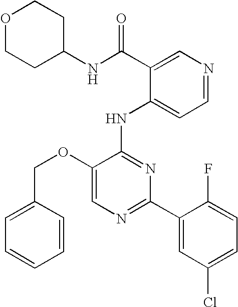 |
| 235 | 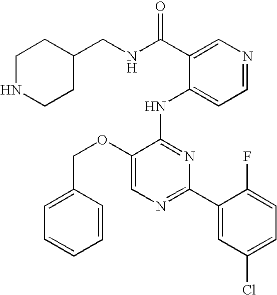 |
| 236 | 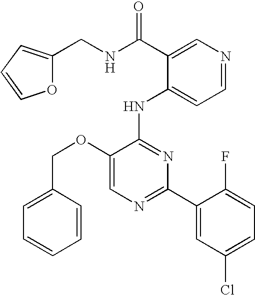 |
| 237 | 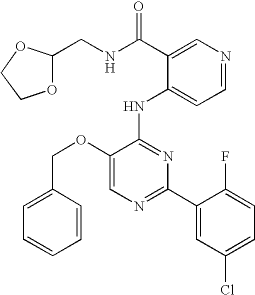 |
| 238 | 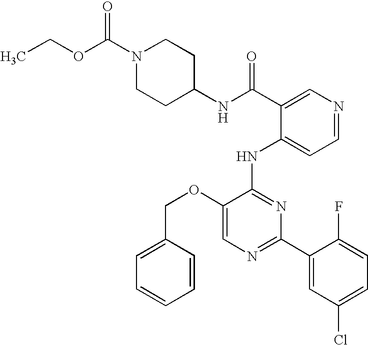 |
| 239 | 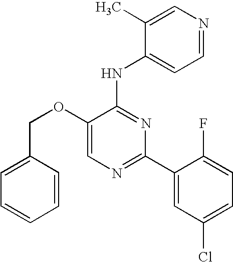 |
| 240 | 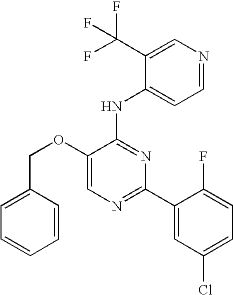 |
| 241 | 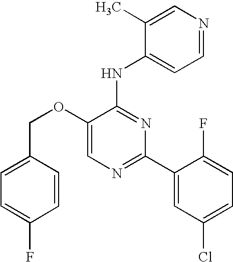 |
| 242 | 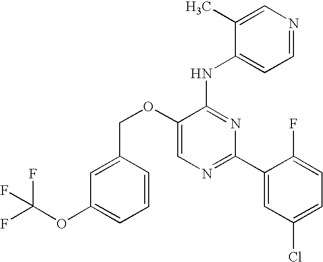 |
| 243 | 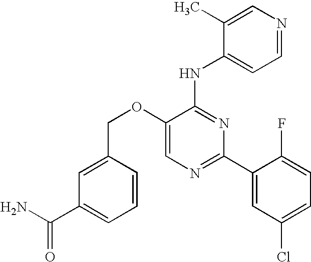 |
| 244 | 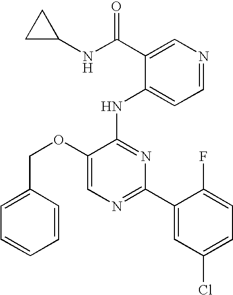 |
| 245 | 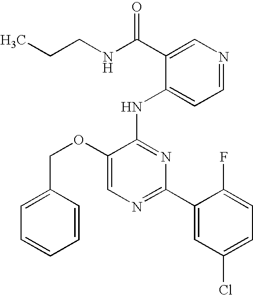 |
| 246 | 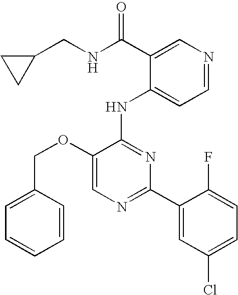 |
| 247 | 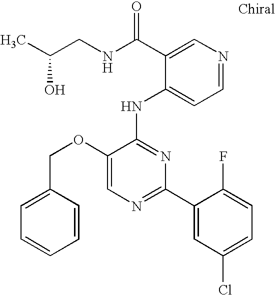 |
| 248 | 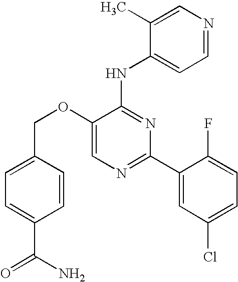 |
| 249 | 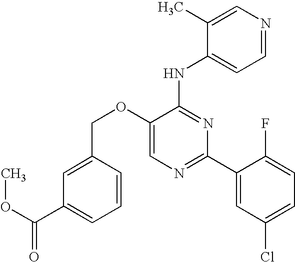 |
| 250 | 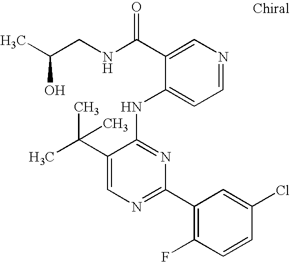 |
| 251 | 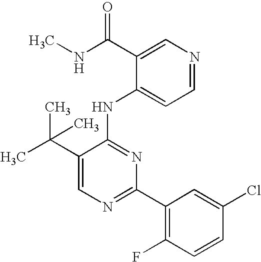 |

Further TGF-β inhibitors for use in the methods of the present invention are represented by formula (5)

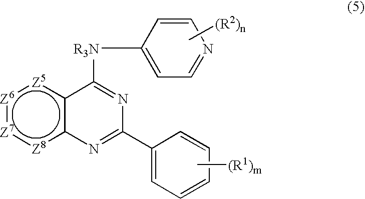

or the pharmaceutically acceptable salts thereof;

wherein each of Z⁵, Z⁶, Z⁷ and Z⁸ is N or CH and wherein one or two Z⁵, Z⁶, Z⁷ and Z⁸ are N and wherein two adjacent Z positions cannot be N;

wherein m and n are each independently 0-3;

wherein two adjacent R¹ groups may be joined to form an aliphatic heterocyclic ring of 5-6 members;

wherein R² is a noninterfering substituent; and

wherein R³is H or CH₃.

Representative compound of formula (5) are listed in the following Table 5.

TABLE 5

| COMPOUND | |
|---|---|
| # | STRUCTURE |
| 252 | 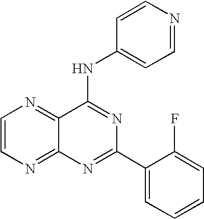 |
| 253 | 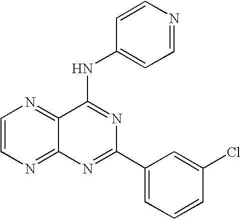 |
| 254 | 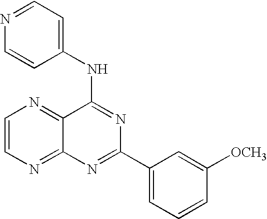 |
| 255 | 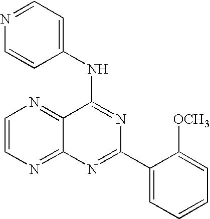 |
| 256 | 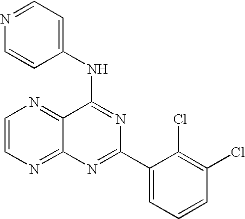 |
| 257 | 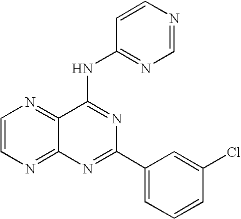 |
| 258 | 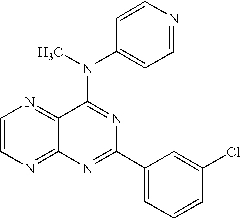 |
| 259 | 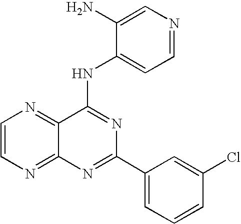 |
| 260 | 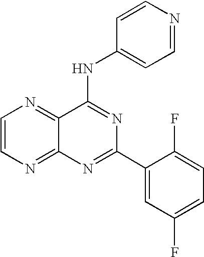 |
| 261 | 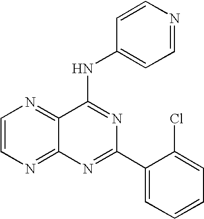 |
| 262 | 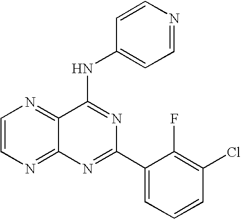 |
| 263 | 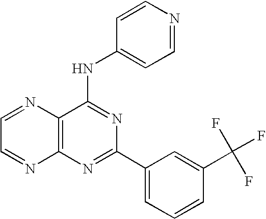 |
| 264 | 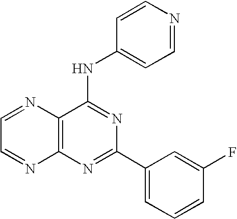 |
| 265 | 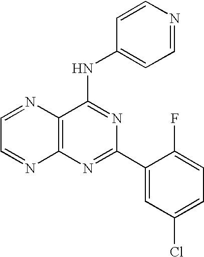 |
| 266 | 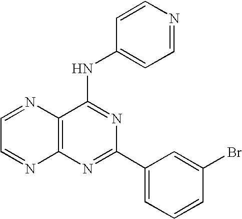 |
| 267 | 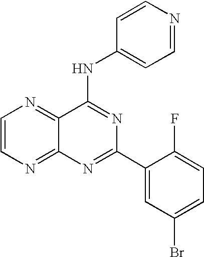 |
| 268 | 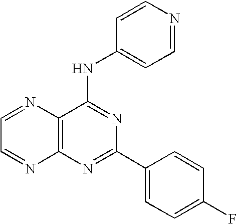 |
| 269 | 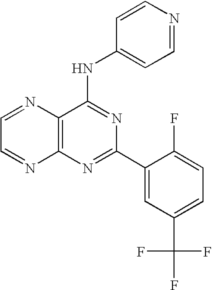 |
| 270 | 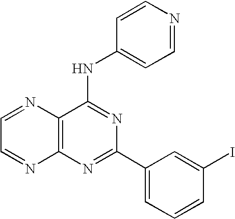 |
| 271 | 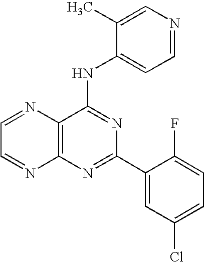 |
| 272 | 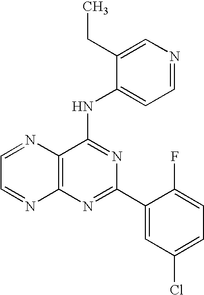 |
| 273 | 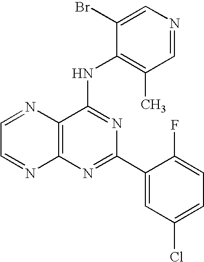 |
| 274 | 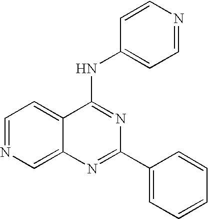 |
| 275 | 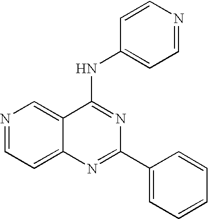 |
| 276 | 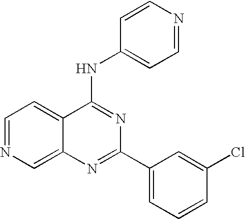 |
| 277 | 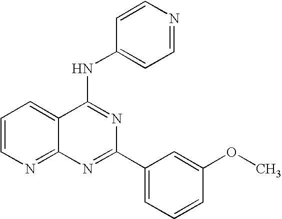 |
| 278 | 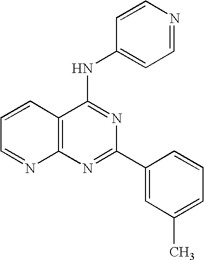 |
| 279 | 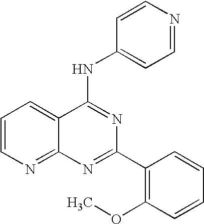 |
| 280 | 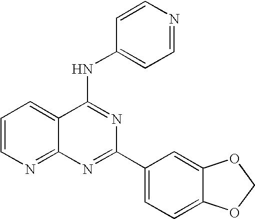 |
| 281 | 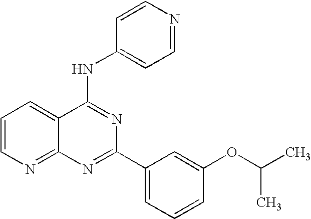 |
| 282 | 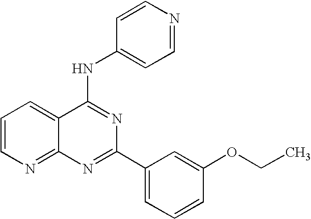 |
| 283 | 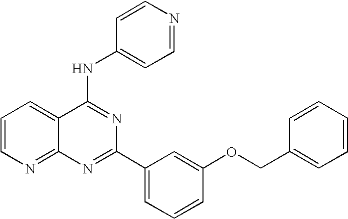 |
| 284 | 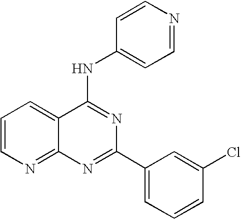 |
| 285 | 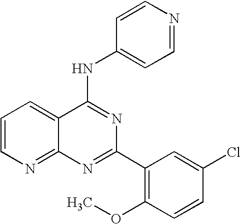 |
| 286 | 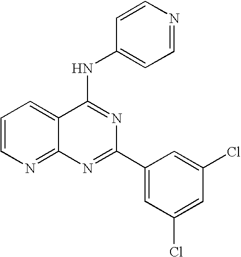 |
| 287 | 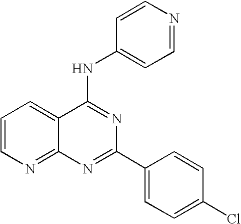 |
| 288 | 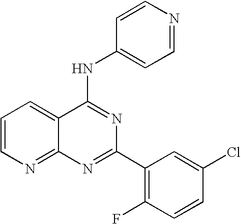 |
| 289 | 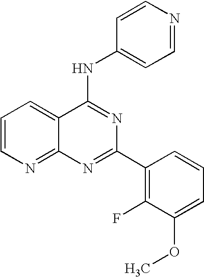 |
| 290 | 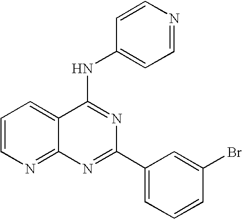 |
| 291 | 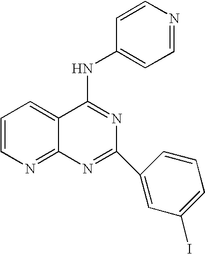 |
| 292 | 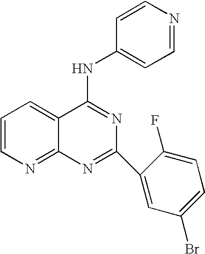 |
| 293 | 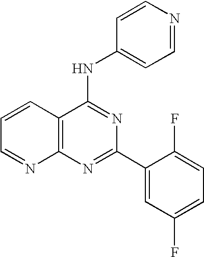 |
| 294 | 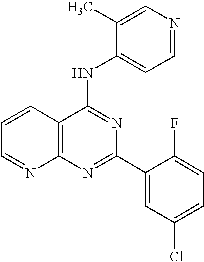 |
| 295 | 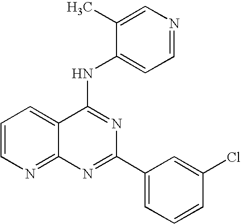 |

The TGF-β inhibitors herein can also be supplied in the form of a “prodrug” which is designed to release the compounds when administered to a subject. Prodrug form designs are well known in the art, and depend on the substituents contained in the compound. For example, a substituent containing sulfhydryl could be coupled to a carrier which renders the compound biologically inactive until removed by endogenous enzymes or, for example, by enzymes targeted to a particular receptor or location in the subject.

In the event that any of the substituents of the foregoing compounds contain chiral centers, as some, indeed, do, the compounds include all stereoisomeric forms thereof, both as isolated stereoisomers and mixtures of these stereoisomeric forms.

Synthesis of Compounds of the Invention

The small molecule compounds of formula (1) of the invention may be synthesized from the corresponding 4-halo-2-phenyl quinazoline as described in Reaction Scheme 1; which may be obtained from the corresponding 4-hydroxyquinazoline as shown in Reaction Scheme 2. Alternatively, the compounds can be prepared using anthranylamide as a starting material and benzoylating the amino group followed by cyclization to obtain the intermediate 2-phenyl-4-hydroxy quinazoline as shown in Reaction Scheme 3. Reaction Schemes 4-6 are similar to Reaction Scheme 3 except that an appropriate pyridine or 1,4-pyrimidine nucleus, substituted with a carboxamide residue and an adjacent amino residue, is substituted for the anthranylimide. The compounds of the invention wherein R¹ is H can be further derivatized to comprise other embodiments of R¹ as shown in Reaction Scheme 7.

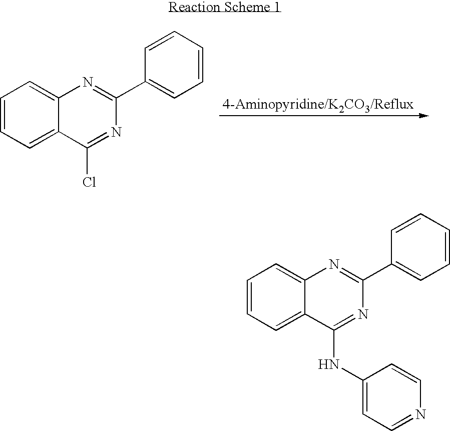

Reaction Scheme 1 is illustrative of the simple conversion of a halogenated quinazoline to compounds of the invention. Of course, the phenyl of the illustration at position 2 may be generalized as R³ and the 4-pyridylamino at position 2 can be generalized to Ar′-L or Ar′—.

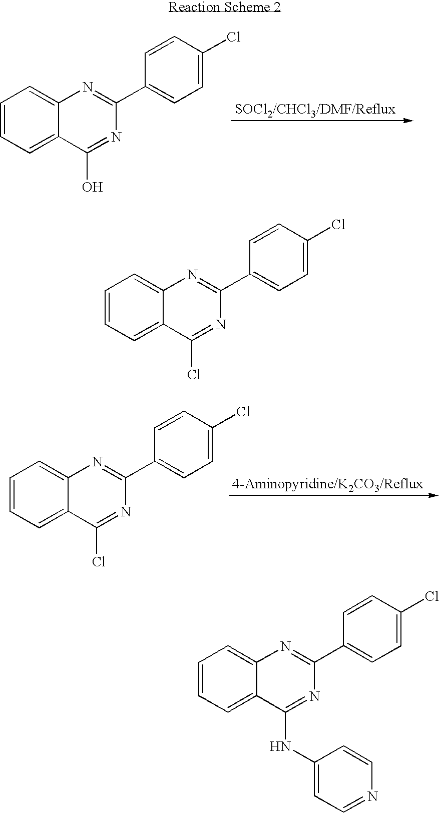

Reaction Scheme 2 can, of course, be generalized in the same manner as set forth for Reaction Scheme 1.

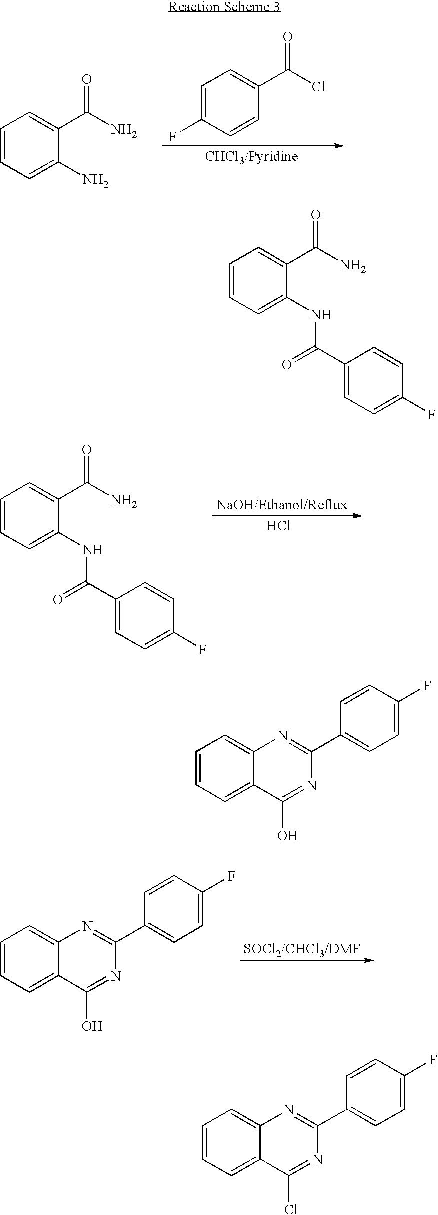

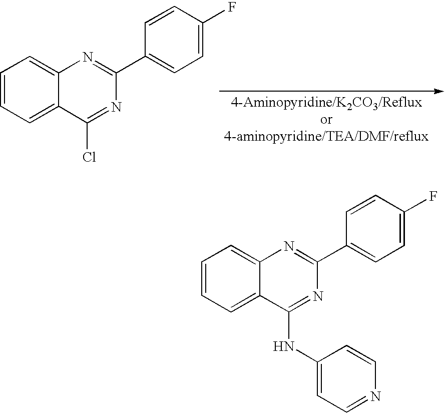

Again, Reaction Scheme 3 can be generalized by substituting the corresponding acyl halide, R³COCl for the parafluorobenzoyl chloride. Further, Ar′ or Ar′-L may be substituted for 4-aminopyridine in the last step.

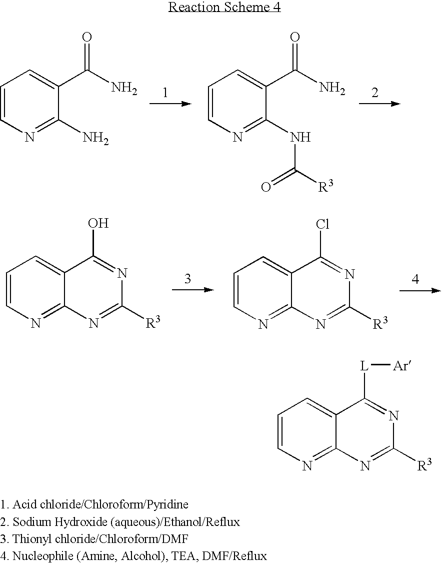

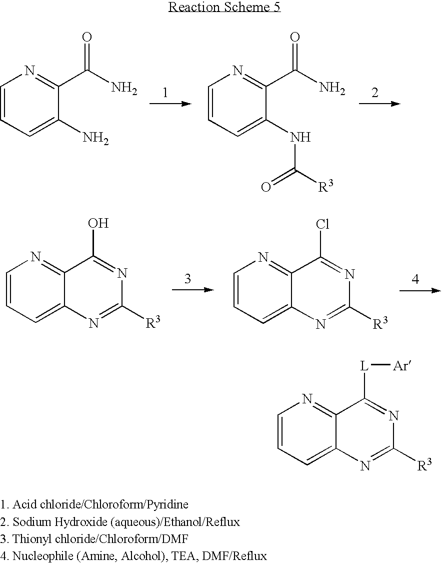

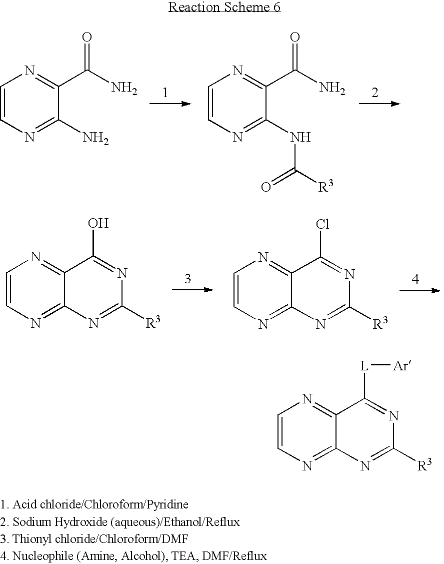

It is seen that Reaction Scheme 1 represents the last step of Reaction Schemes 2-6 and that Reaction Scheme 2 represents the last two steps of Reaction Scheme 3-6.

Reaction Scheme 7 provides conditions wherein compounds of formula (1) are obtained wherein R¹ is other than H.

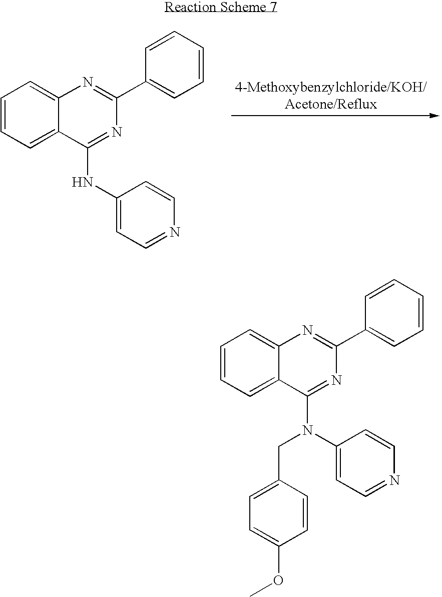

Reaction Scheme 8 is a modification of Reaction Scheme 3 which simply demonstrates that substituents on ring A are carried through the synthesis process. The principles of the behavior of the substituents apply as well to Reactions Schemes 4-6.

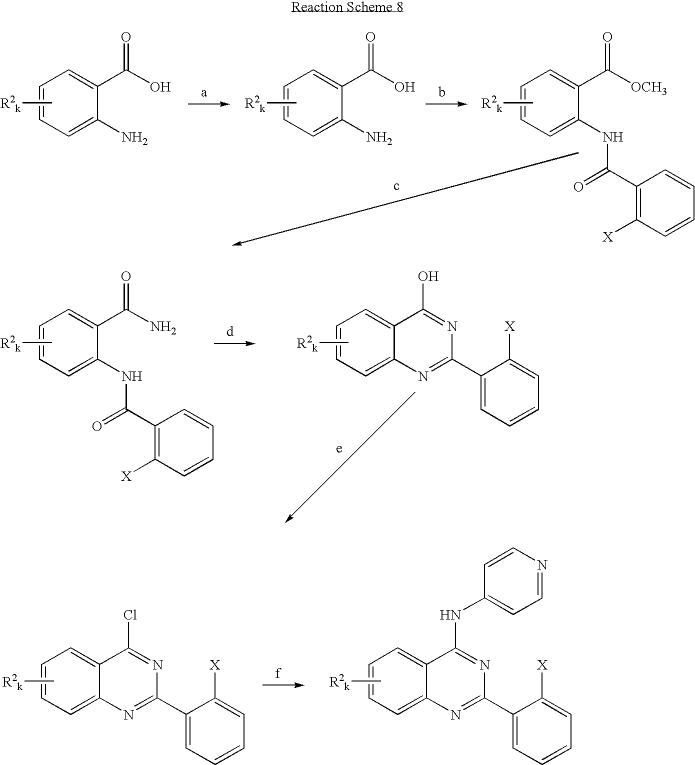

Reaction Scheme 8 shows a modified form of Reaction Scheme 3 which includes substituents R² in the quinazoline ring of formula (1). The substituents are carried throughout the reaction scheme. In step a, the starting material is treated with thionyl chloride in the presence of methanol and refluxed for 12 hours. In step b, the appropriate substituted benzoyl chloride is reacted with the product of step a by treating with the appropriately substituted benzoyl chloride in pyridine for 24 hours. In embodiments wherein X (shown illustratively in the ortho-position) is fluoro, 2-fluorobenzoyl chloride is used as a reagent; where X is (for illustration ortho-chloro), 2-chlorobenzoyl chloride is used.

In step c, the ester is converted to the amide by treating in ammonium hydroxide in an aprotic solvent such as dimethyl formamide (DMF) for 24 hours. The product is then cyclized in step d by treatment with 10 N NaOH in ethanol and refluxed for 3 hours.

The resulting cyclized form is then converted to the chloride in step e by treating with thionyl chloride in chloroform in the presence of a catalytic amount of DMF under reflux for 4 hours. Finally, the illustrated 4-pyridylamino compound is obtained in step f by treating with 4-amino pyridine in the presence of potassium carbonate and DMF and refluxed for 2 hours.

In illustrative embodiments of Reaction Scheme 8, R² may, for example, provide two methoxy substituents so that the starting material is 2-amino-4,5-dimethoxy benzoic acid and the product is, for example, 2-(2-chlorophenyl)-4-(4-pyridylamino)-6,7-dimethoxyquinazoline.

In another illustrative embodiment, R² provides a single nitro; the starting material is thus, for example, 2-amino-5-nitrobenzoic acid and the resulting compound is, for example, 2(2-fluorophenyl)-4-(4-pyridylamino)-5-nitroquinazoline.

Reaction Schemes 4-6 can be carried out in a manner similar to that set forth in Reaction Scheme 8, thus carrying along R² substituents through the steps of the process.

In compounds of the invention wherein R² is nitro, the nitro group may be reduced to amino and further derivatized as indicated in Reaction Scheme 9.

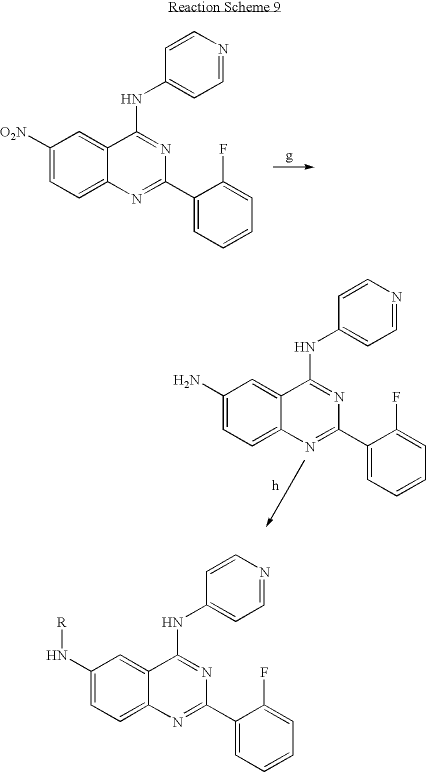

In Reaction Scheme 9, the illustrative product of Reaction Scheme 8 is first reduced in step g by treating with hydrogen and palladium on carbon (10%) in the presence of acetic acid and methanol at atmospheric pressure for 12 hours to obtain the amino compound. The resulting amino compound is either converted to the acyl form (R=acyl) using the appropriate acid chloride in the presence of chloroform and pyridine for four hours, or is converted to the corresponding alkylated amine (R=alkyl) by treating the amine intermediate with the appropriate aldehyde in the presence of ethanol, acetic acid, and sodium triacetoxyborohydride for 4 hours.

While the foregoing exemplary Reaction Schemes are set forth to illustrate the synthetic methods of the invention, it is understood that the substituents shown on the quinazoline ring of the products are generically of the formula (1) as described herein and that the reactants may be substituted accordingly. Variations to accommodate various substituents which represent embodiments of R³ other than the moieties shown in these illustrative examples or as Ar′ in these illustrative examples may also be used. Similarly, embodiments wherein the substituent at position 4 contains an arylalkyl can be used in these schemes. Methods to synthesize the compounds of the invention are, in general, known in the art.

Small organic molecules other than quinazoline derivatives can be synthesized by well known methods of organic chemistry as described in standard textbooks.

Compounds of formula (4) or (5) can be synthesized by methods well known in the art that will be readily apparent for those skilled in the art.

Methods of Treatment

The manner of administration and formulation of the compounds useful in the invention and their related compounds will depend on the nature and severity of the condition, the particular subject to be treated, and the judgment of the practitioner. The particular formulation will also depend on the mode of administration.

Thus, the small molecule compounds of the invention are conveniently administered by oral administration by compounding them with suitable pharmaceutical excipients so as to provide tablets, capsules, syrups, and the like. Suitable formulations for oral administration may also include minor components such as buffers, flavoring agents and the like. Typically, the amount of active ingredient in the formulations will be in the range of about 5%-95% of the total formulation, but wide variation is permitted depending on the carrier. Suitable carriers include sucrose, pectin, magnesium stearate, lactose, peanut oil, olive oil, water, and the like.

The compounds useful in the invention may also be administered through suppositories or other transmucosal vehicles. Typically, such formulations will include excipients that facilitate the passage of the compound through the mucosa such as pharmaceutically acceptable detergents.

The compounds may further be administered by injection, including intravenous, intramuscular, subcutaneous, intraarticular or intraperitoneal injection. Typical formulations for such use are liquid formulations in isotonic vehicles such as Hank's solution or Ringer's solution.

Alternative formulations include aerosol inhalants, nasal sprays, liposomal formulations, slow-release formulations, and the like, as are known in the art.

Any suitable formulation may be used.

If the compounds of the invention are used to counteract loss in β-adrenergic sensitivity resulting from the long-term or excessive use of another therapeutic agent, such as a β2-adrenergic agonist, their route of administration may also depend on the way the other therapeutic agent is administered. For example, β2-agonists used for the treatment of asthma, COPD and other diseases benefiting from the improvement of lung function (in particular from bronchodilation) are often administered as aerosol formulations for inhalation use. Concurrent administration of the compounds of the invention may, therefore, be conveniently performed by using the inhalation route, using the same or different formulation. The compounds of the invention may also be administered in combination with other therapeutic agents, such as natural or synthetic corticosteroids, particularly prednisone and its derivatives, and medications used in the treatment of cardiac diseases, such as congestive heart failure, including, without limitation, brain-derived natriuretic peptide (NBP).

A compendium of art-known formulations is found in Remington's Pharmaceutical Sciences, latest edition, Mack Publishing Company, Easton, Pa. Reference to this manual is routine in the art.

The dosages of the compounds of the invention will depend on a number of factors which will vary from patient to patient. However, it is believed that generally, the daily oral dosage will utilize 0.001-100 mg/kg total body weight, preferably from 0.01-50 mg/kg and more preferably about 0.01 mg/kg-10 mg/kg. The dose regimen will vary, however, depending on the conditions being treated and the judgment of the practitioner.

As implicated above, although the compounds of the invention may be used in humans, they are also available for veterinary use in treating non-human mammalian subjects.

Further details of the invention will be apparent from the following non-limiting examples.

### EXAMPLE 1

TGFβ-RI Inhibitors Counteract Pathologic Changes in the β-Adrenergic Signal Transduction Pathway in Human Bronchial Smooth Muscle Cells (hBSMC) and Cardiomyocytes

Materials and Methods

Materials:

Human recombinant transforming growth factor-β1 (TGFβ1) and Activin A were obtained from R&D System (Minneapolis, Minn.); Porcaterol, propranolol, ICI 118,551 were from Sigma (St. Louis, Mo.); Isoproterenol, forskolin, 3-isobutyl-1-methylxanthine (IBMX) were from Calbiochem (San Diego, Calif.). [5,7-³H]-CGP12177 (specific activity 33 Ci/mmol) was purchased from PerkinElmer Life Sciences (Boston, Mass.). Direct Cyclic AMP (cAMP) EIA kit was from Assay Designs, (Ann Arbor, Mich.). Anti-Smad2/3 mouse monoclonal antibody was purchased from BD Transduction Laboratories (San Diego, Calif.), anti-phospho-Smad2 (Ser465/467) rabbit antiserum was from Cell Signaling Technology. TGFβ receptor I specific inhibitor Compound No. 79 (Table 2) was synthesized by the Medicinal Chemistry Department at Scios, Inc. and dissolved in DMSO as stock.

Cell Culture and Drug Treatment:

Human bronchial smooth muscle cells (hBSMC) were purchased from Clonetics (BioWhittaker, Inc., Walkersville, Md.), and maintained in SmGM-2 containing 5% fetal bovine serum (Clonetics) at 37° C./5% CO₂. All experiments were performed on cells at passages from 6 to 8. For pretreatment, cells were incubated in 1% serum-containing media or serum-free media in the presence or absence of TGFβ1, Activin A and other drugs. Control cells were treated with the appropriate vehicles.

Ventricular cardiomyocytes were isolated from neonatal rat hearts as described before and seeded to fibronectin coated plates in DMEM21 and Coon's F12 with 10% FBS. In particular, single cardiac myocytes were enzymatically isolated from ventricles of 1- to 2-day-old rat pups and maintained in human fibronectin coated plates (Becton Dikenson Labware, Bredford, Mass.) in DMEM21 and Coon's F12 containing 10% fetal bovine serum (FBS) and 1% penicillin-streptomycin as described previously (Henson et al., DNA Cell Biol. 19:757-763 (2000)). Myocytes were used within 24 to 72 hr after isolation.

For experiments, cells were cultured in 24-well plates for cAMP assays and 6-well plates for real-time RT-PCR analyses, Western blotting analyses, and radioligand binding studies. For pretreatment, cells were washed once in serum-free media (SFM) supplemented with 0.1% bovine serum albumin (BSA) and incubated with TGF-β1 in the presence or absence of inhibitors in the same media. Control cells were treated with the appropriate vehicles.

Assay of Cyclic AMP Accumulation:

HBSMC or rat neonatal cardiomyocytes were subcultured in 96-well or 24-well plates for 24 hr to 48 hr, then treated with TGFβ1 (1-2ng/ml), Activin A (10-50 ng/ml) and other drugs in 1% serum-containing or serum-free media. After 24 hr incubation, phosphodiesterase inhibitor, IBMX (200 uM) was added to fresh media for 10-15 min before exposure to either 10 uM procaterol, 1 uM isoproterenol, or 10-50 uM forskolin for 10 min to stimulate cAMP production. The stimulation medium was removed and cells were lysed in 0.1M HCl. cAMP levels were measured using Direct cAMP EIA kit from Assay Designs, Inc. following manufacture's instruction.

Radioligand Binding Assay:

The number of β2-adrenergic receptors on cell surface was determined by radioligand binding using hydrophilic, membrane-impermeable β-adrenergic antagonist [³H]CGP-12177. Intact hBSMCs in 10 cm dish were preincubated with 5 nM [³H]-CGP 12177 in the presence or absence of 20 uM propranolol (to define the amount of nonspecific binding) in SmBM for 1 hour at 37° C. with very gentle shaking. Cells were washed 3 times with ice-cold 1×PBS containing 0.1% Tween-20 (binding buffer) and 3 more times with ice-cold 1×PBS. 400 μl of RIPA buffer containing protease inhibitors was added to the plates and cells were scraped off the plates. Cell lysates were collected and protein concentrations were determined by BCA method (PIERCE). The radioligand bound to the whole cell was quantified by liquid scintillation counter and normalized to the protein concentration.

Quantitative Real-Time RT-PCR:

Total RNA was extracted from cells using Qiagen's RNAeasy kit (Valencia, Calif.), and analyzed by quantitative real time RT-PCR [Gibson UEM, Heid C A and Williams P M. Genome Res. 6, 995-1001, 1996] using an ABI Prism™ 7700 Sequence Detection System (PE Applied Biosystems, Foster City, Calif.). This system is based on the ability of the 5′nuclease activity of Taq polymerase to cleave a nonextendable dual-labeled fluorogenic hybridization probe during the extension phase of PCR. The probe is labeled with reporter fluorescent dye at the 5′ end and a quencher fluorescent dye (6-carboxy-tetramethyl-rhodamine) at the 3′ end. When the probe is intact, reporter emission is quenched by the physical proximity of the reporter and quencher fluorescent dyes. However, during the extension phase of PCR, the nucleolytic activity of the DNA polymerase cleaves the hybridization probe and releases the reporter dye from the probe with a concomitant increase in reporter fluorescence.

Sequence specific primers and probes were designed using Primer Express software (PE Applied Biosystems, Foster City, Calif.). The primers and probe for 18S rRNA were forward 5′-CGGCTACCACATCCAAGGAA-3′ (SEQ ID NO: 1), reverse 5′-GCTGGAATTACCGCGGCT-3′ (SEQ ID NO: 2), and probe 5′-6FAM-TGCTGGCACCAGACTTGCCCTC-TAMRA-3′ (SEQ ID NO:3); for human and rat β1-AR were forward 5′-TGCTACAACGACCCCAAGTG-3′ (SEQ ID NO:4), reverse 5′-AGGTACACGAAGGCCATGATG-3′ (SEQ ID NO: 5), and probe 5′-6FAM-CCATCGCCTCGTCCGTAGTCTCCTT-TAMRA-3′ (SEQ ID NO: 6); for human β2-AR were forward 5′-TGCCGGAGCCCAGATTT-3′ (SEQ ID NO: 7), reverse 5′-ATTCCCATAGGCCTTCAAAGAAG-3′ (SEQ ID NO: 8), and probe 5′-6FAM-AGGATTGCCTTCCAGGAGCTTCTGTGC-TAMRA-3′ (SEQ ID NO: 9); for rat β2-AR were forward 5′-CAACTCTGCCTTCAATCCTCTTATC-3′ (SEQ ID NO: 10), reverse 5′-TGCTAGAGTAGCCGTTCCCATAG-3′ (SEQ ID NO: 11), and probe 5′-6FAM-AGGATTGCCTTCCAGGAGCTTCTGTGC-TAMRA-3′ (SEQ ID 12). Primers were used at a concentration of 200 nM and probes at 100 nM in each reaction. Multiscribe reverse transcriptase and AmpliTaq Gold polymerase (PE Applied Biosystems, Foster City Calif.) were used in all RT-PCR reactions and PCR reactions. RT-PCR parameters were as follows: 48° C. for 30 min (reverse transcription), 95° C. for 10 min (AmpliTaq Gold activation) and 40 cycles of 95° C. for 15 sec, 60° C. for 1 min. Relative quantitation of β1-AR, β2-AR, and 18S mRNA were calculated using the comparative threshold cycle number for each sample fitted to a five point standard curve (ABI Prism 7700 User Bulletin #2, PE Applied Biosystems, Foster City Calif.). Expression levels were normalized to 18S rRNA. The selection of 18S an as endogenous control was based on an evaluation of the ΔC_{T} levels of several housekeeping genes: Cyclophilin A, 18S, GAPDH, and β-Glucuronidase. The ΔC_{T} levels of 18S did not differ significantly between treatment conditions; thus, they were expressed at constant levels between samples (data not shown).

Western Blot Analysis:

After incubation with TGFβ1 and other drugs, cells were washed once with 1×PBS and lysed in 0.2 ml/plate cold RIPA buffer (phosphate buffered saline, pH 7.4, 1% NP-40, 0.5% sodium deoxycholate, 0.1% sodium dodecylsulfate, 1 mM sodium orthovanadate, 1 mM NaF, 1 mM β-glycerolphosphate, 1 uM okadaic acid, 10 ng/ml aprotinin, 10 ng/ml leupeptin, 1 mM phenymethylsulfonyl fluoride). Samples were clarified by centrifugation (4° C., 10 min, 15,000×g), and protein concentration was determined by BCA method (PIRCE). Lysates with equal amounts of total cell protein (15-20 ug) were separated on 10% SDS-NuPAGE (Invitrogen) and then transferred to nitrocellulose membrane. The membrane was blocked in 3% nonfat dry milk/TBST (10 mM Tris-HCl pH 7.5, 150 mM NaCl, 0.1% Tween 20) for 1 hr, and probed with anti-Smad2/3 mouse monoclonal antibody [Transduction Laboratories (S66220); 1:500 dilution in 3% milk/TBST], or anti-phospho-Smad2 (Ser465/467) rabbit antiserum [Cell Signaling (3101S); 1:500 dilution in 3% BSA/TBST] at 4° C. overnight. Donkey anti-mouse HRP [Santa Cruz (SC-2314); 1:2000 dilution] or donkey anti-rabbit HRP [Santa Cruz (SC-23130; 1:2000 dilution] were used as secondary antibody. Immunoreactivity was detected with chemiluminescence reagent (Santa Cruz) and visualized by exposing to x-ray film (Kodak).

Results

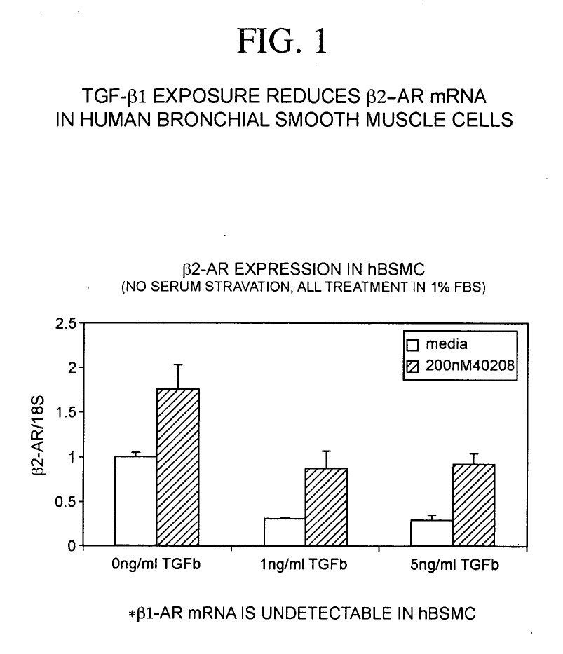

Human bronchial smooth muscle cells (hBSMC) were treated with TGFβ1, and β2AR mRNA analyzed by real-time quantitative PCR as described above. The results shown in FIG. 1 demonstrate that TGFβ1 exposure significantly reduces β2AR.

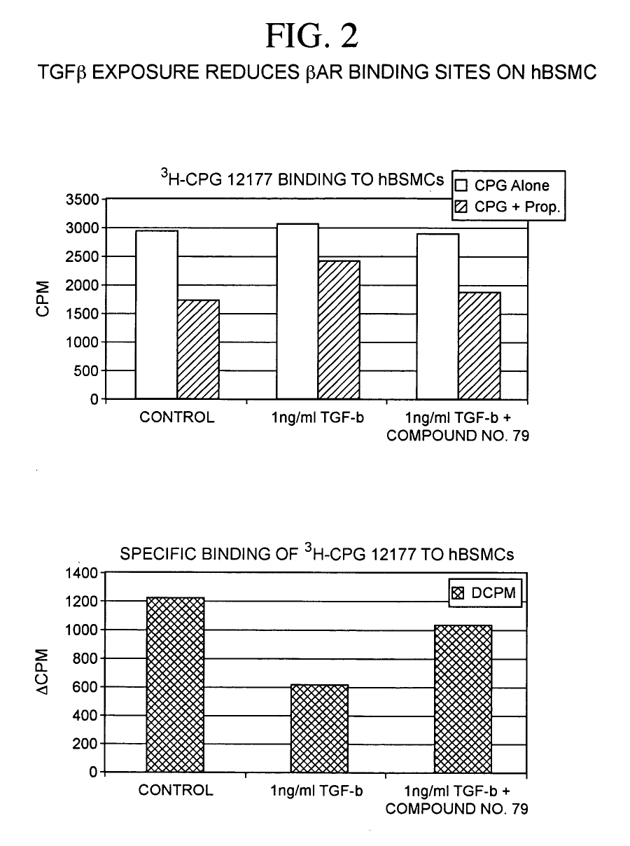

hBSMC were treated with TGFβ1, and the number of β2-adrenergic receptors on cell surface was determined by radioligand binding using hydrophilic, membrane-impermeable β-adrenergic antagonist [3H]CGP-12177. As shown in FIG. 2, TGFβ1 exposure reduces βAR binding sites on hBSMC.

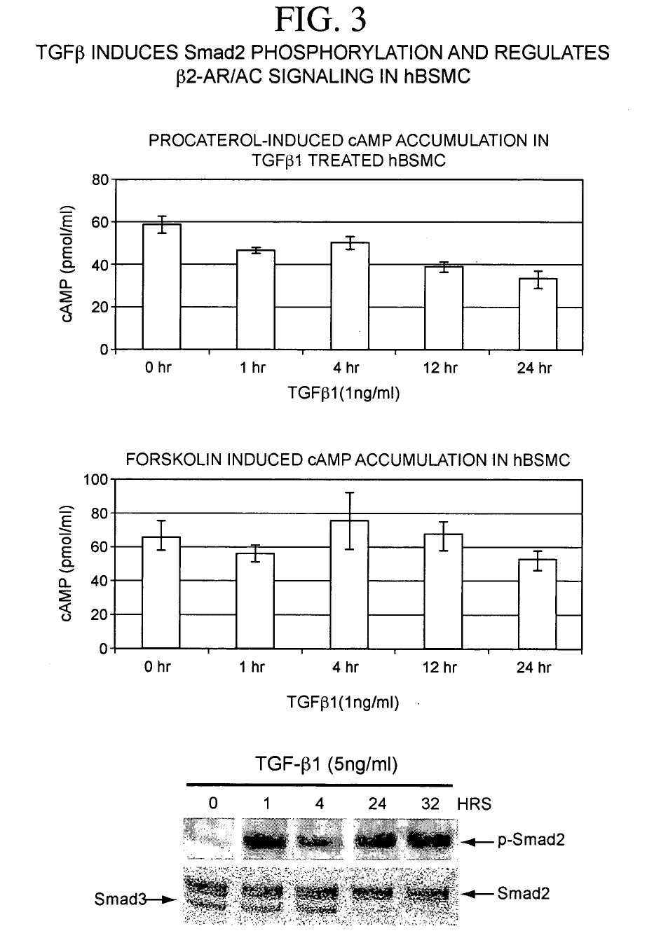

Cyclic AMP (cAMP) accumulation was measured as described above. FIG. 3 shows the time course of the effect of TGFβ1 on procaterol-induced and forskolin-induced cAMP accumulation in hBSMC. Procaterol is a specific agonist of β2AR, and forskolin activates adenylyl cyclase (AC), and both procaterol and forskolin can induce cAMP production in the cells. As shown in FIG. 3, TGFβ1-induced loss of β2AR response happened after 12 hr and was more profound at 24 hr or later, while TGFβ1-affected AC activity and signaling was only observed 24 hr later, to a lesser extent.

These results demonstrate that TGFβ1 induces Smad2 phosphorylation and regulates β2AR/AC signaling in hBSMC.

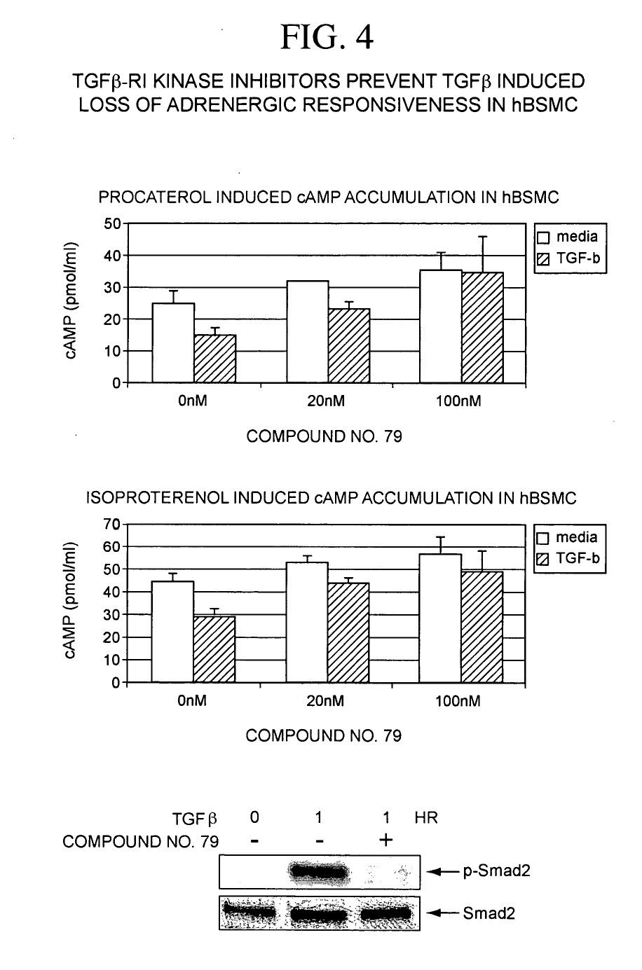

hBSMC were treated with procaterol and isoproterenol as described above, in the presence of a representative non-peptide small molecule inhibitor of TGFβ-R1. As shown in FIG. 4, the inhibitor prevented TGFβ-induced loss of adrenergic responsiveness in hBSCM. In addition, the inhibitor prevented TGFβ-induced Smad2 phosphorylation and loss of adrenergic responsiveness in hBSMC.

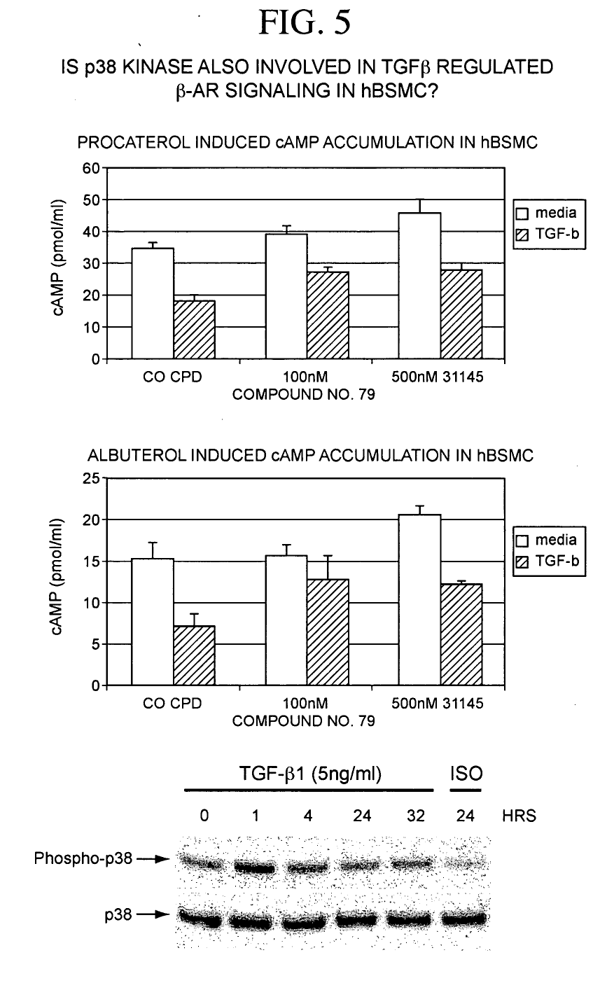

FIG. 5 shows TGFβ1 treatment-induced p38 phosphorylation in hBSMC, and TGFβ1-induced loss of β2AR, which could be partially reversed by a p38 inhibitor.

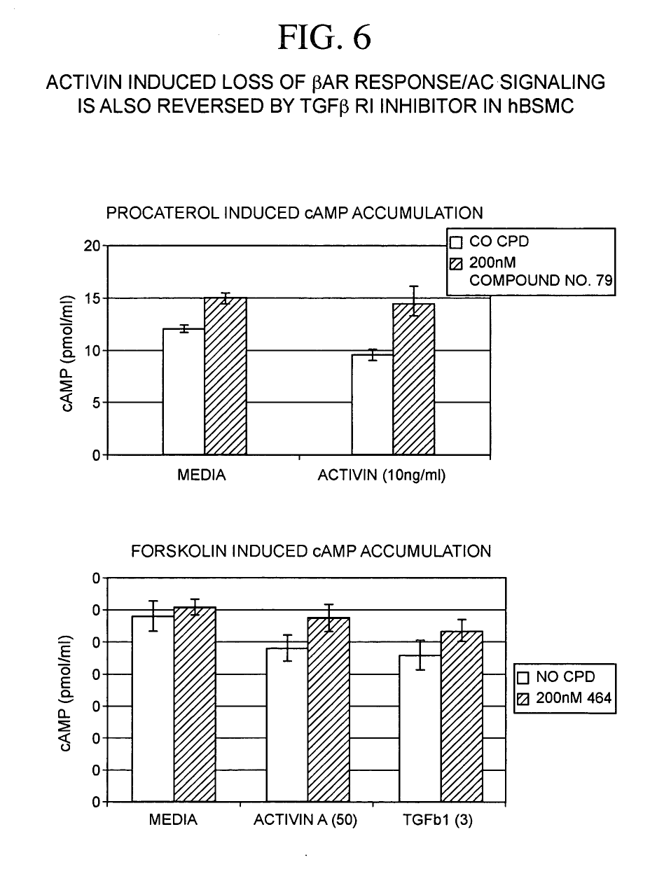

FIG. 6 shows that activin A at higher concentration also causes loss of βAR response as well as reduced AC activity in hBSMC. These effects were reversible by TGF-β-R1 inhibitors.

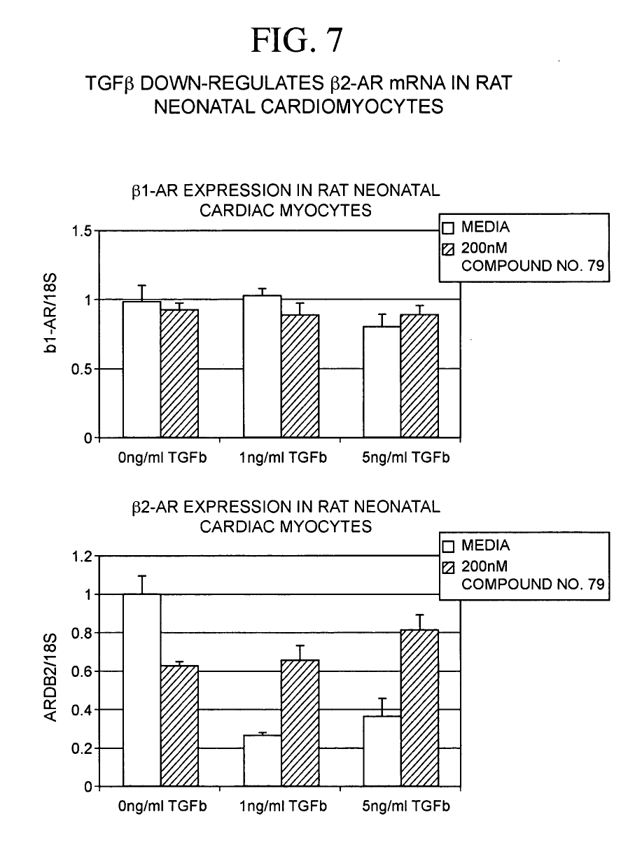

Rat neonatal cardiac myocytes were treated with TGFβ1 and β1AR expression monitored as described above. The results shown in FIG. 7 show that TGFβ1 downregulates β2AR mRNA in rat neonatal cardiomyocytes.

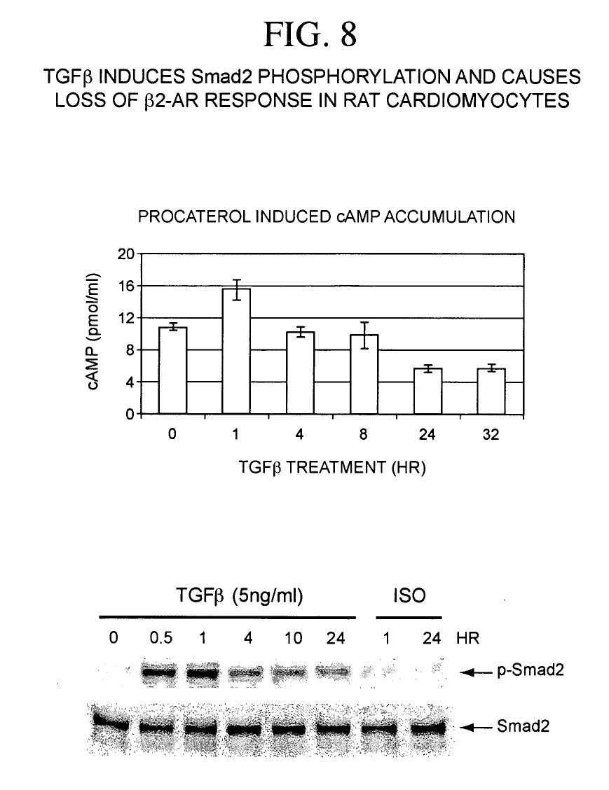

In rat neonatal cardiac myocytes treated with TGFβ1 cAMP accumulation was measured as described above. The results shown in FIG. 8 show that TGFβ1 induces Smad2 phosphorylation and causes loss of β2AR response.

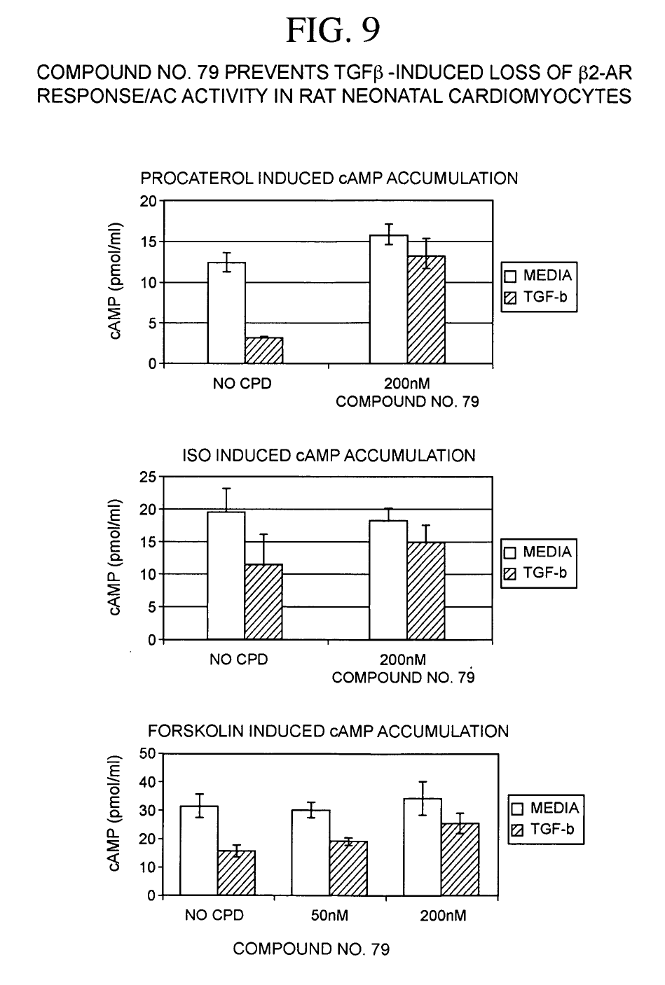

As shown in FIG. 9, a representative small molecule compound of formula (1) prevents TGFβ1-induced loss of β2AR response and AC activity in rat neonatal cardiomyocytes.

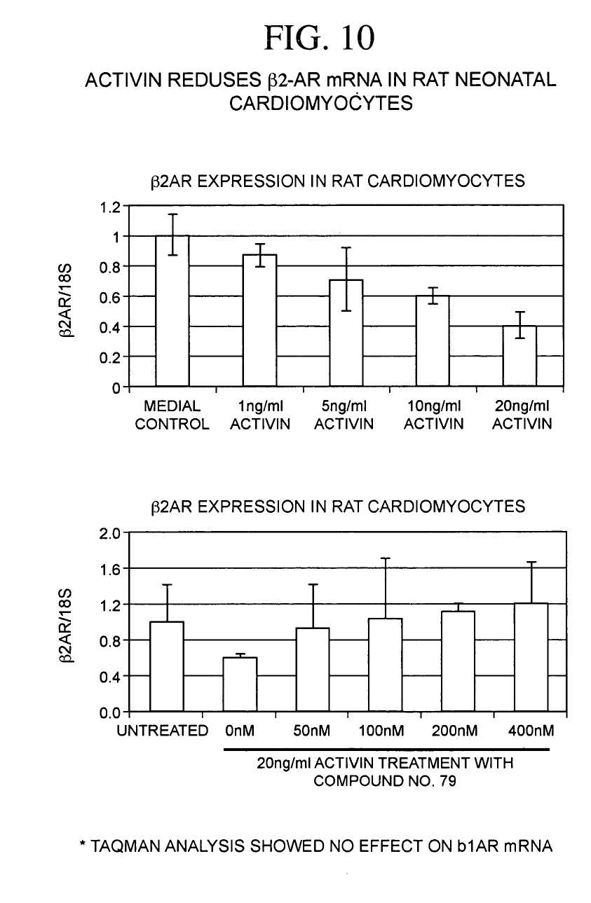

As shown in FIG. 10, Activin down-regulated β2AR mRNA in rat neonatal cardiomyocytes, and this down-regulation can be prevented by a representative small-molecule TGFβ1 inibitor.

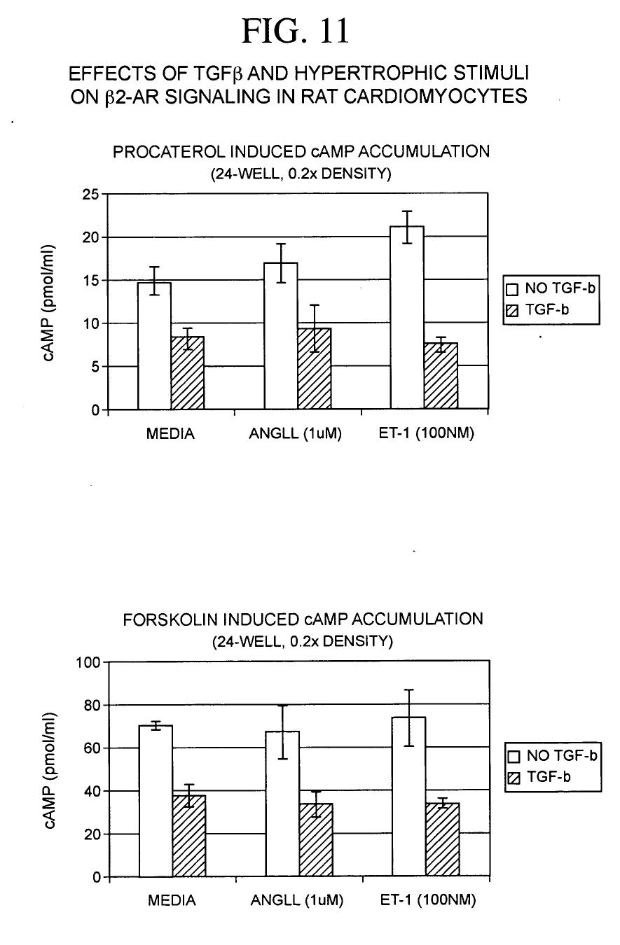

Rat cardiomyocytes were cultured and treated with procaterol and forskolin, respectively, as described above. As shown in FIG. 11, subsequent treatment with activin A and IL-1β, respectively, induces loss of β2AR response/AC activity.

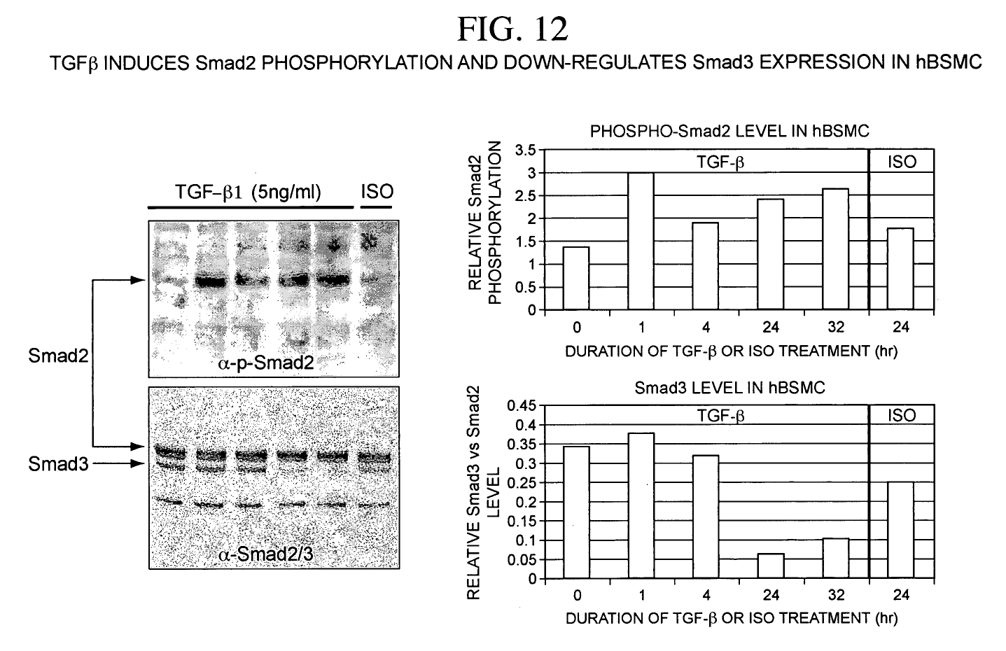

Next, TGFβ-induced Smads signaling was investigated in hBSMC cell culture. Western blot analysis was performed and phosphor-Smad2 and Smad3 levels were determined as describe above. The results shown in FIG. 12 demonstrate that TGFβ1 induces Smad2 phosphorylation and down-regulated Smad3 expression in hBSMC.

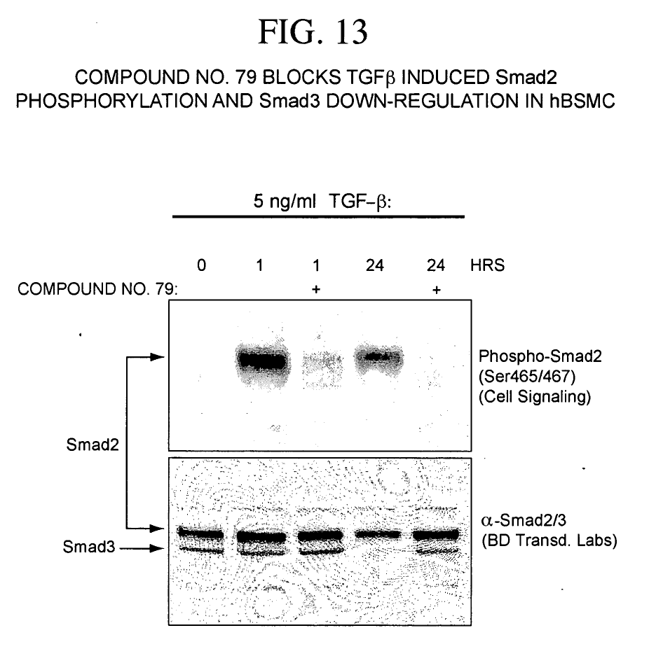

FIG. 13 shows that a representative compound of formula (1) blocks TGFβ1-induced Smad2 phosphorylation and Smad3 down-regulation in hBSMC.

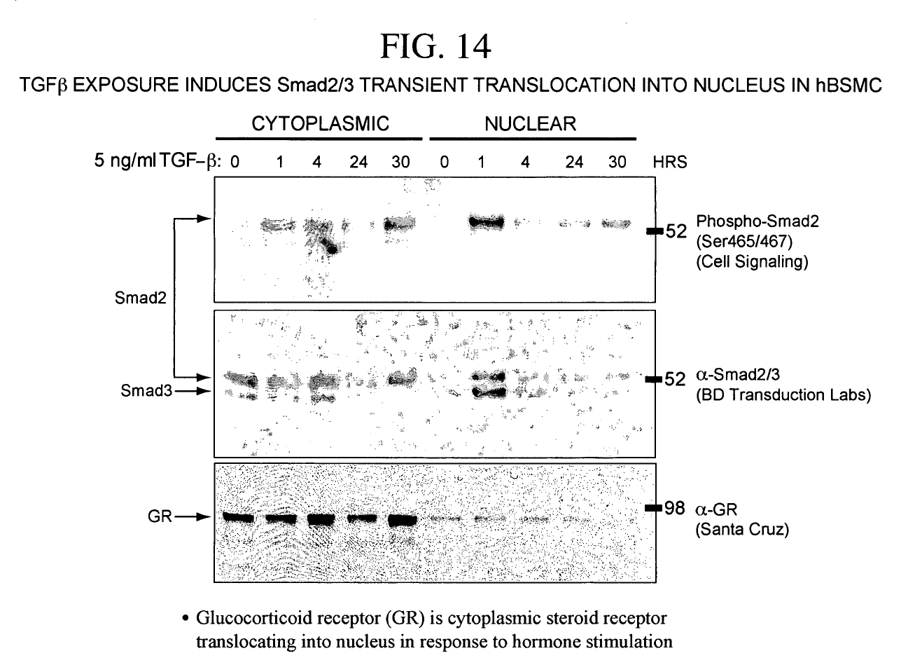

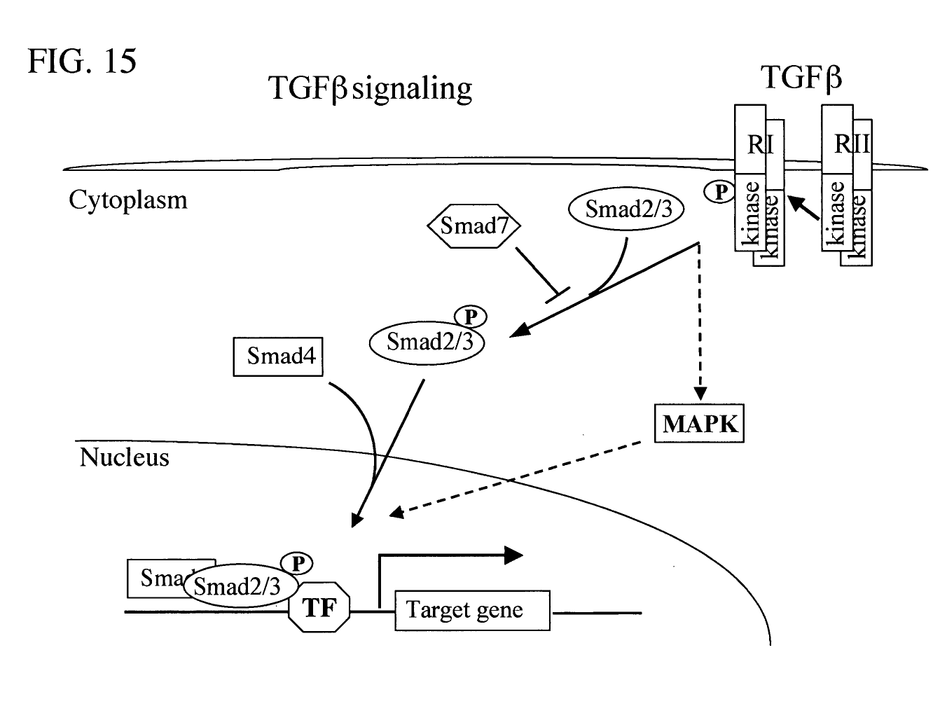

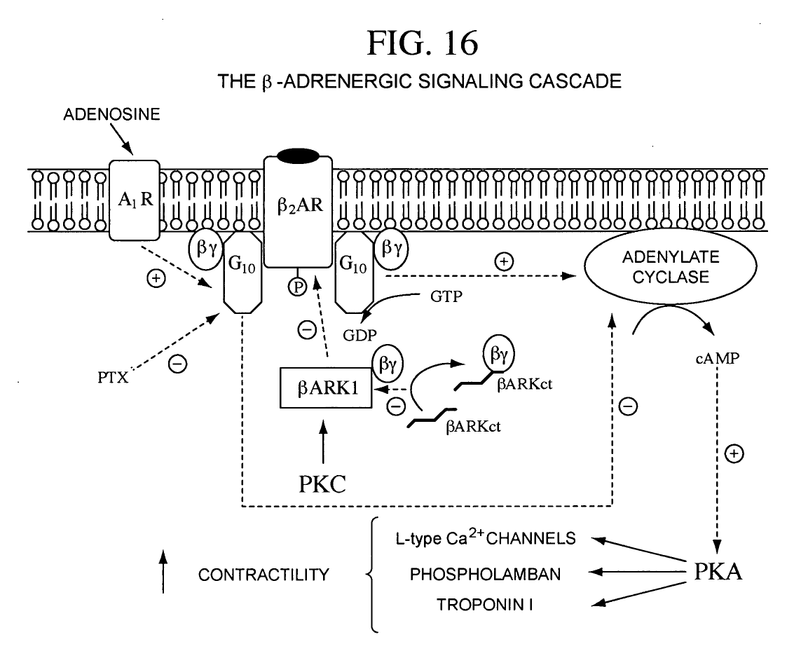

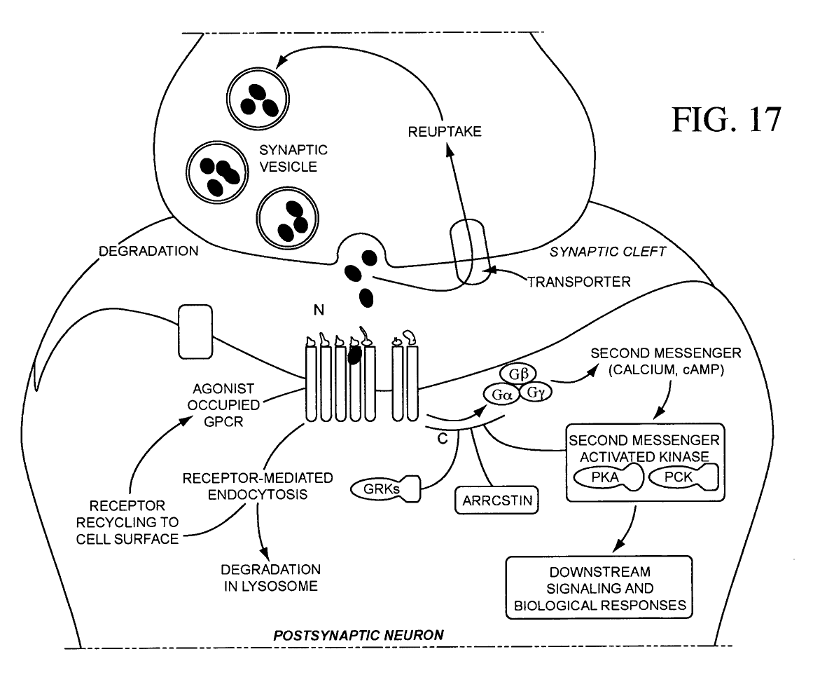

FIG. 14 shows that TGFβ1 exposure induces Smad2/3 transient translocation into the nucleus in hBSMC.

In conclusion, the experiments described in the present example have demonstrated that TGFβ1 induces loss of β2AR response and reduces AC activity in both hBSMC and rat cardiomyocytes. TGFβ1 exerts its function through activation of Smad2/3 transcription factors. The results discussed above have additionally shown that representative TGFβ-RI inhibitors are able to block TGFβ1 effects by blocking Smad2/3 activation. In addition, activin was found to have similar effects, which could be reversed by a representative TGFβ-RI inhibitor.

### EXAMPLE 2

Effect of TGFβ-RI Inhibitors on TGF-β Signaling in Cardiomyocytes

Materials and Methods

Reagents:

The reagents were obtained from the same sources as described in Example 1. (L)-form, cell permeable JNK inhibitor I were from Calbiochem (San Diego, Calif.). TGF-β type I receptor (TGFβ-RI) inhibitor Compound No. 79 (see Table 2) and a p38α MAP kinase inhibitor were synthesized by the Medicinal Chemistry Department at Scios, Inc. and dissolved in DMSO as stocks. Compound No. 79 has an IC50 of ˜37 nM against in vitro TGFβ-RI kinase activity with specificity of >100-fold against TGFβ-RII receptor and at least 20-fold over members of a panel of related protein kinases (data not included).

Cardiomyocytes Culture and Treatment

Cardiomyocytes were cultured and treated as described in Example 1.

Assay of Cyclic AMP Accumulation

Subsequent to treatment with TGF-β1 (1-2 ng/ml), cardiomyocytes (˜1×10⁵ cells/well in 24-well plates) were incubated with phosphodiesterase inhibitor, IBMX (200 μM) for 30 min in SFM. Cells were then exposed to either procaterol (10 μM), forskolin (10-50 μM), or isoproterenol (Iso, 1 μM) for 10 min to allow for accumulation of cAMP. In some experiments, a selective β1-AR antagonist, CGP-20712A (200 nM), or a selective β2-AR antagonist, ICI 118, 551 (200 nM) was preincubated with the myocytes before Iso treatment to stimulate specific β2-AR or β1-AR mediated cAMP accumulation, respectively. The incubations were terminated by removal of the medium. Cells in each well were lysed in 150 μl of 0.1 M HCl at room temperature (RT) for 30 min. Intracellular cAMP contents were measured using the Direct cAMP EIA kit following manufacture's instruction, and the cAMP levels were calculated in pmol/ml.

Radioligand Binding Assay

The radioligand binding assay was performed as described in Example 1. To define the nonspecific binding to β2-AR, 50 nM CGP-20712A were used.

Real-Time RT-PCR

Real-time RT-PCR was performed as described in Example 1, and included the use of the following additional sequence specific primers and probes:

For rat Smad3 were forward 5′-CAGCACACAATAACTTGGACCTACAG-3′, (SEQ ID NO: 13), reverse 5′-AACTCGCTGGTTCAGCTCGTA-3′ (SEQ ID NO: 14), and probe 5′-6FAM-AGCCGGCCTTTTGGTGCTCCA-TAMRA-3′ (SEQ ID NO: 15); for rat βARK-1/GRK2 were forward 5′-TGGGCTGCATGCTCTTCA-3′ (SEQ ID NO: 16), reverse 5′-GCGGTCAATCTCATGCTTGTC-3′ (SEQ ID NO: 17), and probe 5′-6FAM-CCTTCCGGCAGCACAAGACCA-TAMRA-3′ (SEQ ID NO: 18); for rat AC5 were forward 5′-ACCGCCAATGCCATAGACTT-3′ (SEQ ID NO: 19), reverse 5′-CACCTTCAGCGCCACCTT-3′ (SEQ ID NO: 20), and probe 5′-6FAM-CCCAGTGCCCTGAGCATGCGA-TAMRA-3′ (SEQ ID NO: 21); for rat AC6 were forward 5′-GCCTGTCCCGCAGTATCGT-3′ (SEQ ID NO: 22), and reverse 5′-GAACACAAGCAGAACCGAGAAGA-3′ (SEQ ID NO: 23), and probe 5′-6FAM-CACGGGTGCACAGCACGGCT-TAMRA-3′ (SEQ ID NO: 24); for rat Giα-1 were forward 5′-CGGGAGTACCAGCTGAACGA-3′ (SEQ ID NO: 25), and reverse 5′-TGGGTTGGGATGTAATTTGGTT-3′ (SEQ ID NO: 26), and probe 5′-6FAM-CGGCGTACTACCTGAATGACTTGGACAGAAT-TAMRA-3′ (SEQ ID NO: 27); for rat Giα-2 were forward 5′-TGCGGACCCGTGTGAAG-3′ (SEQ ID NO: 28), and reverse 5′-CGCTGACCACCCACATCA-3′ (SEQ ID NO: 29), and probe 5′-6FAM-AGGCATCGTCGAAACACACTTCACCTTC-TAMRA-3′ (SEQ ID NO: 30); for rat Giα-3 were forward 5′-GCTTCATATTACCTAAATGATTTGGATAGA-3′ (SEQ ID NO: 31), and reverse 5′-CCACAATGCCTGTAGTCTTCACTCT-3′ (SEQ ID NO: 32), and probe 5′-6FAM-TCCCAGACCAACTACATTCCAACTCAGCA-TAMRA-3′ (SEQ ID NO: 33).

Western Blot Analysis

Western blot analysis was carried out as described in Example 1, and included the use of the following additional primary antibodies: Smad2/3 monoclonal antibody (BD Transduction Laboratories, San Diego, Calif.); anti-phospho-Smad2 (Ser465/467) rabbit antiserum (Cell Signaling Technology, Inc., Beverly, Mass.); antibodies for Actin (sc-1616) and GRK-2 (sc-13143) (Santa Cruz Biotechnology, Santa Cruz, Calif.); anti-Gsα, anti-Giα-1, anti-Giα3 antibodies (Calbiochem, San Diego, Calif.). The binding of primary antibodies was followed by incubation for 1 hour at RT with the secondary horseradish peroxidase (HRP) conjugated goat anti-mouse or goat anti-rabbit antibody (Santa Cruz Biotechnology). Immunoreactivity was detected with chemiluminescence reagents and visualized by exposing to x-ray film (Kodak).

Immunofluorescence Staining

The nuclear translocation of Smad proteins in response to TGF-β1 was determined by immunofluorescence staining with monoclonal anti-Smad2/3 antibody (BD Transduction Labarotories) and anti-Smad4 antibody (sc-7966) (Santa Cruz Biotechnology). Briefly, myocytes cultured on Lab-Tek chamber slides (Nalge Nunc International, Naperville, Ill.) coated with fibronectin and gelatin were fixed with 4% paraformaldehyde in PBS for 5 min, then penetrated with 0.1% saponin/1% normal goat serum (NGS) in PBS for 15 min. After blocking nonspecific binding with 5% NGS/0.05% saponin in PBS for 15 min, the slides were incubated with primary antibody in 1% NGS in PBS at 4° C. overnight. After another washing and blocking, slides were incubated with a biotinylated anti-mouse antibody for 30 min, followed by fluorescein isothiocyanate (FITC)-conjugated-avidin (Vector, Burlingame, Calif.) for 30 min. Slides were dried and mounted in Vectashield (Vector). Samples were analyzed by fluorescence microscopy.

Statistical Analysis

Data examined were expressed as mean+S.E. Student's t test was used for comparison of paired groups. A P value of less than 0.05 was considered to be statistically significant.

Results

TGF-β1 Induces Loss of β2-AR Response in Rat Cardiomyocytes

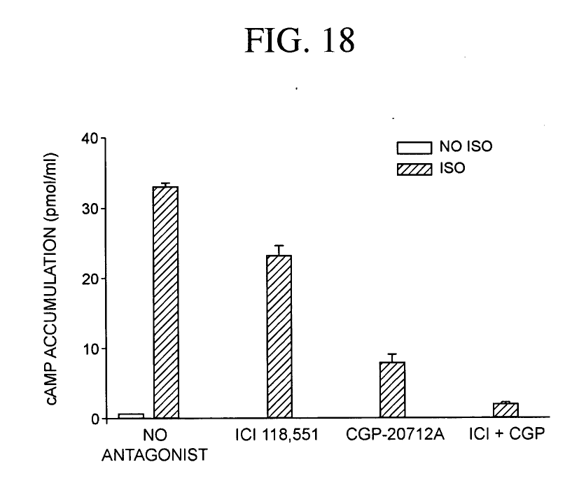

Initial experiments employed the nonselective β-AR agonist isoproterenol (1 μM) to define maximal cAMP accumulation in rat cardiomyocytes. In agreement with previous findings (Steinberg, Cir Res. 85:1101-1111 (1999); Sabri et al., Cir Res. 86:1047-1053 (2000)), the response is attenuated by ˜75% with 200 nM CGP-20712A (β1-AR antagonist), by ˜25% with 200 nM ICI 118, 551 (β2-AR antagonist), and blocked by more than 90% in the presence of both (FIG. 18). These results indicate that the stimulation of cAMP accumulation was mediated through the combined action of β1- and β2-ARs. A β2-AR specific agonist procaterol (10 μM) also substantially increased cAMP accumulation in rat cardiomyocytes.

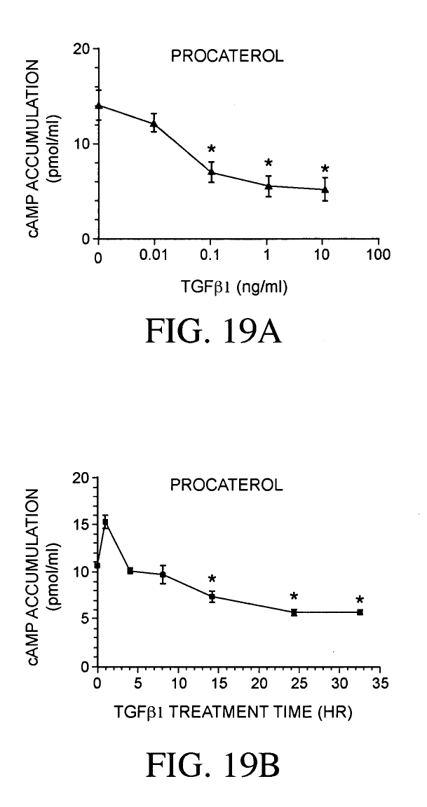

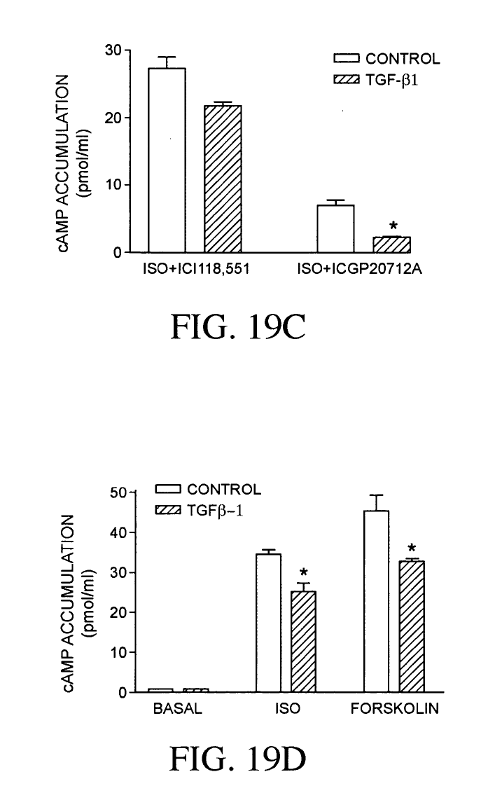

Pre-treatment of cardiomyocytes with TGF-β1 for 24 hr caused a concentration-dependent decrease in the subsequent stimulation of cAMP accumulation by the β2-AR agonist procaterol (10 μM). The maximal effect was attained at 1 ng/ml TGF-β1, resulting in a 59±5.2% reduction (n=5 independent experiments) (FIG. 19A). The effect of TGF-β1 was time dependent; the maximal decrease of cAMP accumulation was observed by 24 hr (FIG. 19B). TGF-β1 also significantly decreased cAMP accumulation (˜65%) when β2-ARs were selectively stimulated by isoproterenol in the presence of CGP-20712A (FIG. 19C). Interestingly, TGF-β1 pretreatment of myocytes for 24 hr only caused a small reduction (16.5±3.1%) (n=3 independent experiments) in β1-AR mediated cAMP accumulation measured by stimulation with isoproterenol in the presence of ICI 118, 551 (FIG. 19C). In addition, TGF-β1 exposure decreased cAMP accumulation stimulated by the direct adenylyl cyclase (AC) activator forskolin (25 μM) (FIG. 19D), which suggests that TGF-β1 induced loss of β-adrenergic sensitivity involves alteration in AC activity. Together, these results indicate that TGF-β1 treatment of cardiomyocytes causes diminution of β-AR response to agonist stimulation primarily due to reduced β2-AR response; moreover, the decreased AC activity contributes at least in part.

TGF-β1 Down-Regulates β2-AR Steady-State mRNA Levels and Receptor Number

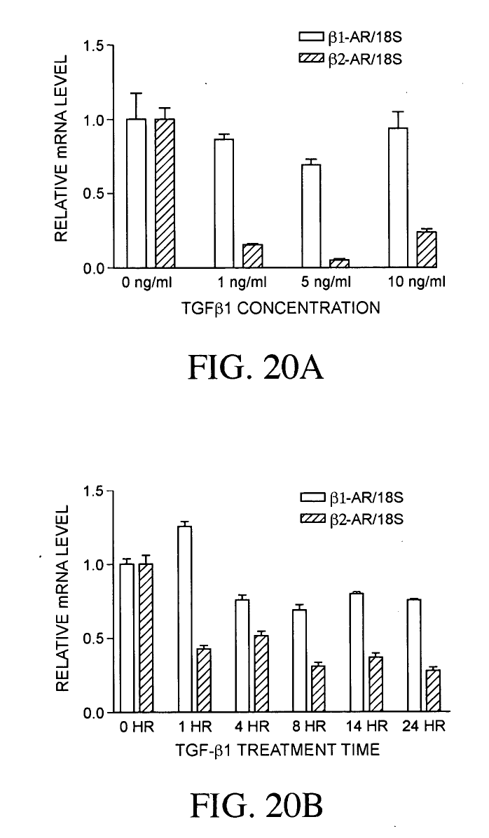

TGF-β1 has been shown to modulate β-AR receptor and function in various cell types through down-regulation of β2-AR mRNA and protein (Iizuka et al., J. Mol. Cel. Cardiol. 26:435-440 (1994); Nogami et al. Am. J. Physiol. 266:L187-191 (1994); Mak et al., Naunyn. Schmiedebergs. Arch. Pharmacol. 362:520-525 (2000)). To investigate whether a change in β-ARs mRNA can be detected, real-time RT-PCR analyses were performed. TGF-β1 pretreatment for 24 hr decreased β2-AR mRNA levels dramatically (FIG. 20A). Consistent with the functional assay, TGF-β1 exposure did not significantly alter β1-AR mRNA levels in cardiomyocytes. Time course study further revealed that the suppression of β2-AR mRNA by TGF-β1 occurred as early as 1 hr after treatment, indicating the regulation of β2-AR gene transcription is a rapid event in rat neonatal cardiomyocytes (FIG. 20B). These results show that TGF-β1 down-regulates β2-AR message levels, suggesting a mechanism for possible decreased receptor expression in cardiomyocytes.

TGF-β1 Effects on the Expression of β-Adrenergic Signaling Molecules

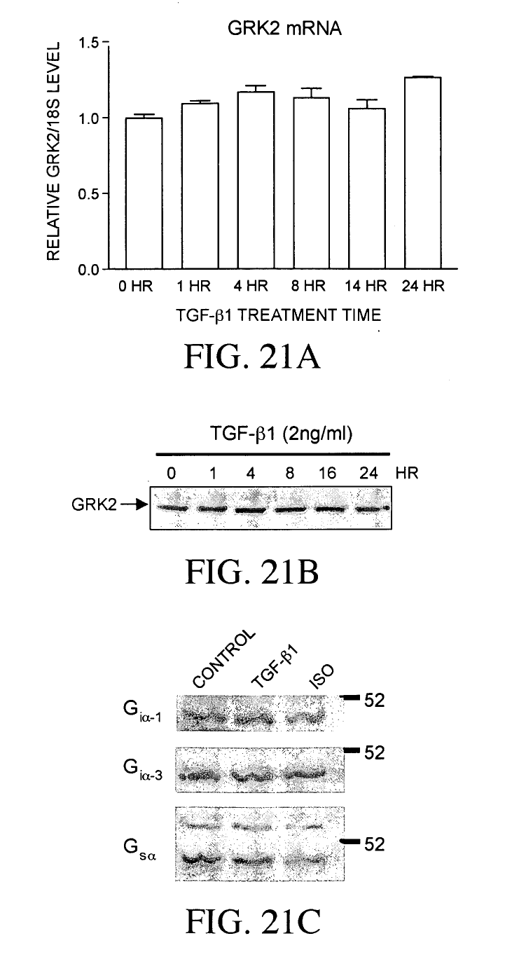

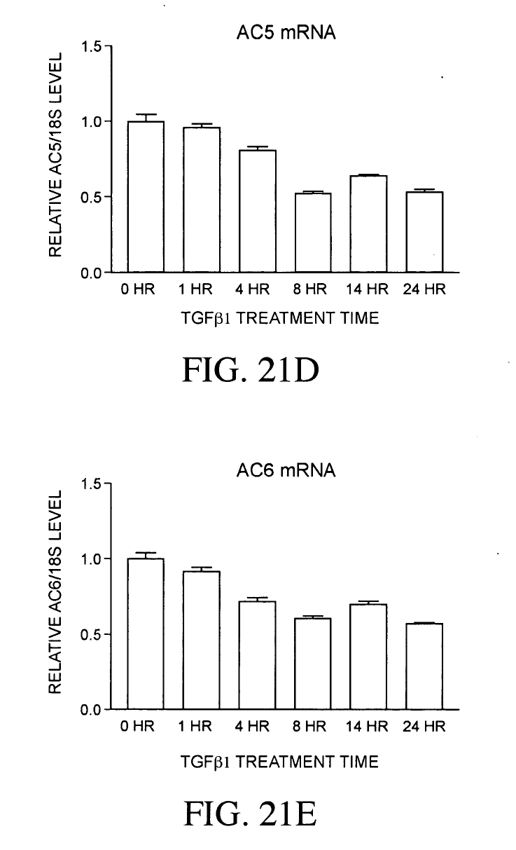

To examine whether there are changes in the expression of other β-AR signaling molecules that could contribute to the altered cAMP response to β-agonists in TGF-β1 treated cardiomyocytes, we examined mRNA and/or protein levels using real-time PCR and Western analyses, respectively. Several candidates that mediate β-AR signaling in cardiomyocytes were tested, including β-AR kinase-1 (βARK1, also known as GRK2), Gs, Gi, AC5 and AC6. Representative data are shown in FIG. 5. TGF-β1 exposure did not alter the expression of βARK1, Gsα, Giα-1, nor Giα-3 in cardiomyocytes at either message or protein levels (FIGS. 21A-C, data not shown). In contrast, the mRNA levels of AC5 and AC6 showed significant reduction by TGF-β1 in a time-dependent manner (FIGS. 21D-E), suggesting the decrease in forskolin-induced AC activity in TGF-β1 treated cardiomyocytes could result from reduced AC5 and AC6 expression.

TβR1 Kinase Inhibitor Blocks TGF-β1-Activated Smad Signaling in Cardiomyocytes

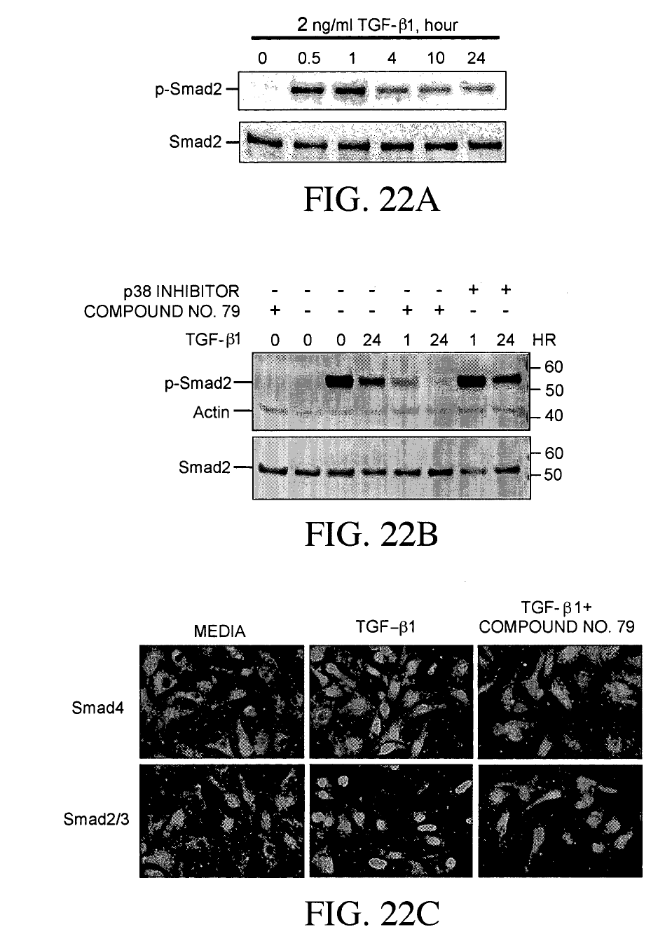

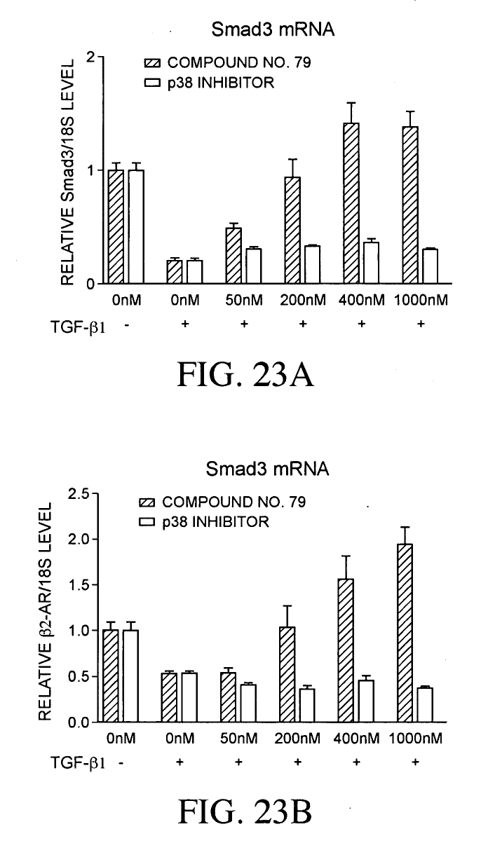

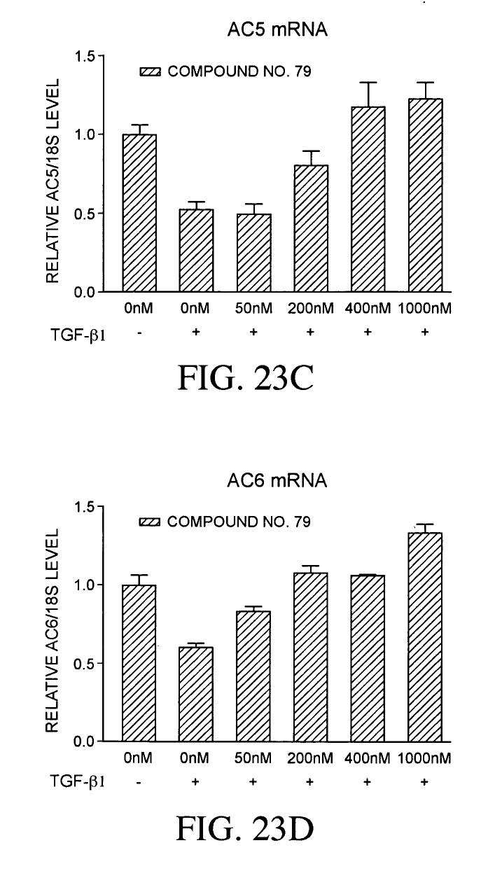

To decipher the signaling pathway(s) responsible for TGF-β1 induced loss of β2-AR response, first the potential signaling events initiated by TGF-β1 in cultured cardiomyocytes were investigated. Incubation of myocytes with TGF-β1 induced rapid activation of Smad signaling. Serine phosphorylation of Smad2 protein peaked at 1 hr, and was maintained for a period of 24 hr with minimal change of total Smad2 protein level (FIG. 22A). A similar phosphorylation profile was observed for Smad3 protein in cardiomyocytes treated with TGF-β1 (data not shown). To determine whether TGF-β1 activated Smad signaling is dependent on TβRI kinase activity, a selective small molecule inhibitor Compound No 79 was used. Pre-incubation with 400 nM Compound No. 79 significantly blocked Smad2 phosphorylation/activation induced by TGF-β1 at both 1 hr and 24 hr (FIG. 6B). In contrast, pre-incubation with a specific p38 kinase inhibitor Compound No. 79 did not influence TGF-β1 induced Smad2 phosphorylation (FIG. 22B). Immunofluorescence staining with specific monoclonal antibodies to Smad2/3 or Smad4 revealed the predominant cytosolic localization of Smad2/3 and Smad4 in resting cardiomyocytes (FIG. 22C). Upon stimulation with TGF-β1 for 1 hr, the fluorescence staining was dramatically increased in the nucleus, indicating the nuclear translocation of Smad2/3 and Smad4. Again, Compound No. 79 at 400 nM completely abolished TGF-β1 induced Smad2/3 and Smad4 translocation into the nucleus in these cells. In addition, we found that TGF-β1 treatment down-regulated Smad3, but not Smad2 mRNA in cardiomyocytes within 24 hr. This was also blocked by Compound No. 79 in a dose-dependent fashion (FIG. 23A, data not shown). In contrast, inhibitory Smad7 mRNA was up-regulated by TGF-β1 treatment in cardiomyocytes (data not shown), representing a negative feedback loop. Down-regulation of Smad3 could represent another negative feedback loop of TGF-β signaling, as reported in several systems (Poncelet et al., Kidney International 56:1354-1365 (1999); Zhao and Geverend, Biochem. Biophys. Res. Commun. 294:319-323 (2002)), and suggests possible differential roles of Smad3 than Smad2 in transducing TGF-β signal to regulate gene expression in cardiomyocytes.

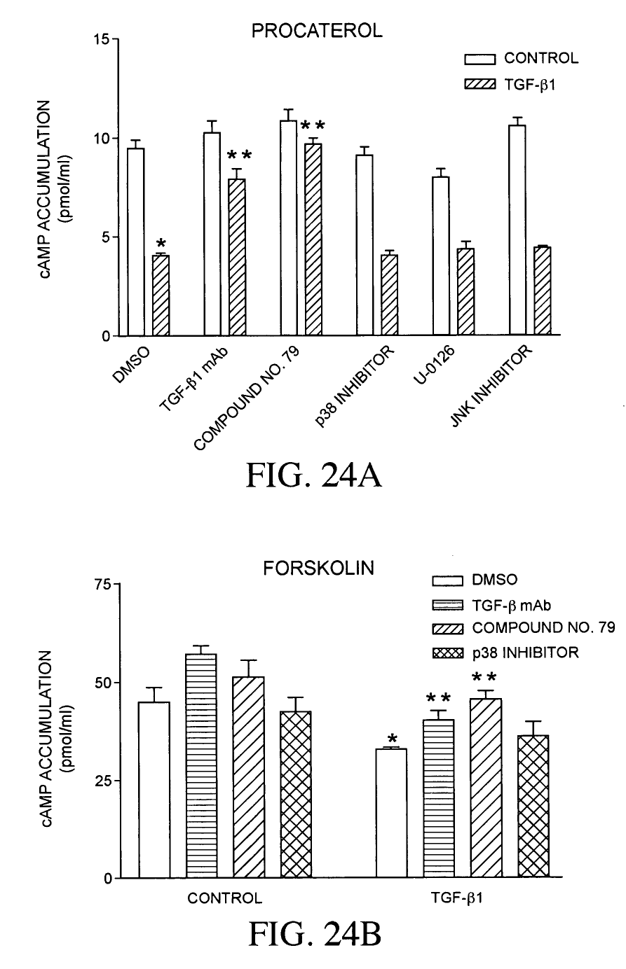

The effect of TGF-β1 on MAP kinase pathways was also examined. Specific inhibitors of MEK1/2 (U0126), c-Jun N-terminal kinase (JNK) (cell permeable peptide inhibitor I) and p38 MAP kinase were used in functional assays to examine their roles in TGF-β1 regulation of β2-AR response. No significant effects of these compounds were observed (FIG. 24A). These data indicate that TGF-β RI kinase dependent Smad signaling is activated in rat cardiomyocytes upon stimulation by TGF-β1, and is probably one of the major signal transduction pathways that potentially mediate the cellular actions of TGF-β1 in these cells.

Compound No. 79 Blocks TGF-β1 Induced Down-Regulation of β2-AR Expression and Function

We next investigated the effect of Compound No. 79 co-treatment with TGF-β1 on β2-AR gene and protein expression levels. Cultured cardiomyocytes were incubated with 5 ng/ml of TGF-β1 in the absence or presence of Compound No. 79 for 24 hr. TGF-β1 induced down-regulation of β2-AR mRNA was reversed by Compound No. 79 in a dose-dependent manner (FIG. 23B). At high doses (400-1000 nM) of Compound No. 79, mRNA levels of β2-AR in TGFβ1 treated cells are higher than that in control cells, suggesting that basal TGF-β signaling in resting cardiomyocytes is inhibited by TGFβRI kinase inhibitor Compound No. 79. In contrast, the p38 inhibitor had no effect on β2-AR mRNA level. In addition, decreased AC5 and AC6 mRNA levels in TGF-β1 treated cardiomyocytes were also inhibited by Compound No. 79 in a dose-dependent fashion (FIGS. 23C, D).

Functional analysis of β2-AR response to procaterol stimulation after 24 hr TGF-β1 exposure showed increased cAMP accumulation in cardiomyocytes in the presence of 200 nM Compound No. 79 or a neutralizing anti-TGFβ pan-specific monoclonal antibody compared to vehicle control (FIG. 24A). In contrast, Compound No. 79, U0126, or JNK inhibitor I, did not affect β2-AR mediated cAMP accumulation in TGF-β1 treated cardiomyocytes, indicating that the major MAP kinase pathways are not responsible for TGF-β1 modulation of β2-AR function. Furthermore, isoproterenol or forskolin induced cAMP accumulation in TGF-β1 treated cardiomyocytes was preserved by pre-incubation with 200 nM Compound No. 79 or TGF-β neutralizing antibody (FIG. 24B). Taken together, these data show that TβRI kinase inhibitor Compound No. 79 blocks TGF-β/Smad signaling and abrogates TGF-β1 induced suppression of β2-AR gene expression and function in cardiomyocytes.

Discussion

The present study demonstrates that TGF-β1 treatment induces β-adrenergic functional desensitization resulting in reduced cAMP accumulation in response to β-agonists (both β2-specific procaterol and non-specific β-agonist isoproterenol) in rat cardiomyocytes. The effect was more dramatic on β2-AR response, with maximum ˜60% decrease in procaterol stimulated cAMP production at 24 hr. The TGF-β1 effect is concentration and time dependent, and the effective concentrations of TGF-β1 were in the physiological range (Li et al., Circulation 98:II-144-II-160 (1998)). A clear down-regulation of β2-AR mRNA levels by TGF-β1 was observed. Radioligand binding experiments showed a trend to decrease β2-AR receptor binding sites in TGF-β1 treated cardiomyocytes, and the reduction can be explained by down-regulation of. Interestingly, TGF-β1 did not alter β1-AR mRNA nor receptor levels, suggesting the decreased β1-AR-mediated cAMP accumulation in TGF-β1 treated cardiomyocytes probably involves other mechanism(s), such as reduced AC activity.

Indeed, TGF-β1 treatment of cardiomyocytes decreased the ability of forskolin, a direct AC activator, to augment cAMP accumulation in intact cells. It has further been shown that the expression of two major cardiac AC isoforms, AC5 and AC6 (Hanoune and Defer, Annu. Rev. Pharmacol. Toxicol. 41:145-174 (2001)), was also suppressed by TGF-β1 in a time-dependent manner, which could contribute to the decreased AC activity in membranes derived from TGF-β1 treated cells (Nair et al., J. Cel. Physiol. 164:232-239 (1995)). In contrast, expression of other signaling molecules downstream of the β-ARs (Gsα, Giα-1, -2, -3, and βARK1) was not altered. Therefore, TGF-β1 induced loss of β2-AR responsiveness in cardiomyocytes may be due to combined actions of decreased β2-AR protein level and altered AC activity.

Compound No. 79, just as the other compounds generically or specifically disclosed in the present application, belongs to a new class of potent, selective small molecule inhibitors of the TGF-β RI kinase. Using this inhibitor, the data presented herein demonstrate that Smad signaling pathway mediates TGF-β1 modulation of β2-AR expression and function in rat cardiomyocytes. TGF-β1 induced Smad2/3 activation and nuclear translocation, as well as basal phosphorylation of Smad2, were blocked by incubation with Compound No. 79 (FIG. 22B), suggesting that there is basal TGF-β signaling present in cultured resting cardiomyocytes due to autocrine mechanism. This phenomenon is reflected in β2-AR gene expression where treatment with Compound No. 79 not only restored β2-AR mRNA levels reduced by TGF-β1 but at high concentration it also increases β2-AR level greater than that in untreated cultures (FIG. 23). In agreement, it has also been observed that Compound No. 79 increased the basal cAMP levels in cardiomyocytes when used at higher concentration.

A large body of evidence has demonstrated that the cardiac response to β-AR stimulation decreases in chronic heart failure in human and in animal models. Studies also suggest that there is a positive correlation between increased plasma catecholamine levels and the degree of the diminution of the β-AR response (Bristo, Lancet 1998, supra). Despite many similarities, β1-AR and β2-AR have markedly different chronic effects on cardiac hypertrophy and survival attributable to the dual coupling of β2-AR to Gs and Gi proteins (Ziao et al., Circ. Res. 85:1092-1100 (1999)). In general, β2-AR appears to be protective while β1-AR over-stimulation is detrimental. Transgenic overexpression of cardiac β1-AR at low level results in cardiac hypertrophy and heart failure (Engelhardt et al., Proc. Natl. Acad. Sci. USA 93:16701-16708 (1999)). In contrast, over-expression of β2-AR at moderate level enhanced biochemical and in vivo cardiac function (Minano et al., Science 264:582-586 (1994) and Liggett et al., Circulation 101:1707-1714 (2000)). Furthermore, studies in cultured rat cardiomyocytes suggest that β2-AR can elicit survival signals on agonist stimulation, whereas β1-AR stimulation activates only apoptotic pathways (Communal et al., Circulation 100:2210-2212 (1999) and Zhu et al., Proc. Natl. Acad. Sci. USA 98:1607-1612 (2001)). Given these findings, selective reactivation of cardiac β2-AR may provide catecholamine-dependent inotropic support without cardiotoxic consequences. Indeed, heart-specific expression of β2-AR by adenoviral delivery in several experimental models has brought about a significant improvement in myocardial β-AR signaling and in ventricular function (Akhter et al., Proc. Natl. Acad. Sci. USA 94:12100-12105 (1997); Maurice et al., J. Clin. Invest. 104:21-29 (1999) and Shah et al., Circulation 101:408-414 (2000)).

In the present study, it has been demonstrated that TβRI kinase inhibitor Compound No. 79 selectively increases β2-AR expression and response to β-agonists in TGF-β1 treated cardiomyocytes. TGF-β1 has been shown to play a key role in other aspects of HF, such as hypertrophy and fibrosis. Other studies using Compound No. 79 also show that it is able to block TGF-β mediated fibrosis in several in vitro and in vivo models. The combined characteristics of TβRI kinase inhibitors such as Compound No. 79 present a new treatment paradigm for chronic heart failure.

### EXAMPLE 3

Alteration of β-AR Binding Sites by TGF-β1 and Compound No. 79 in Cardiomyocytes

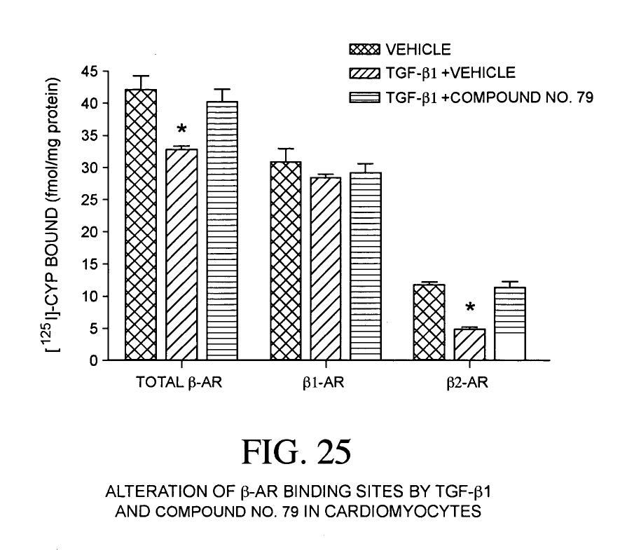

Cardiomyocytes were treated with vehicle of 500 nM Compound No. 79 in the presence or absence of TGF-β1 for 24 hours. Membranes were then prepared and binding of 100 pm [¹²⁵]-CYP was measured for 2 hours at 23° C. using 8 μg membrane protein, and expressed as fmol/mg protein. Binding in the presence of 100 nM CGP-20712A was defined as binding to β2-AR, while binding in the presence of 100 μM propranolol was defined as non-specific binding. Subtraction of β2-AR binding from total binding was defined as β1-AR binding. The results are shown in FIG. 25 (p<0.05 vs. vehicle).

What is claimed is:

1. A method for counteracting a pathologic change in the β-adrenergic signal transduction pathway, comprising administering to a mammalian subject in need an effective amount of a compound capable of inhibiting TGF-β signaling through a TGF-β receptor

2. The method of claim 1 wherein the TGF-β receptor is a TGFβ-R1 receptor kinase.

3. The method of claim 2 wherein said compound is capable of specific binding to a TGFβ-R1 receptor kinase.

4. The method of claim 2 wherein said compounds preferentially inhibits a biological activity mediated by a TGFβ-R1 receptor kinase.

5. The method of claim 1 wherein the pathologic change is selected from the group consisting of (a) a reduction in the mRNA level of a β-adrenergic receptor, (b) a reduction in the number of β-adrenergic receptor binding sites, (c) TGF-β-induced down-regulation of Smad3 expression, and (d) loss in β-adrenergic sensitivity.

6. The method of claim 5 wherein the loss in β-adrenergic sensitivity is associated with the administration of a β-adrenergic agonist.

7. The method of claim 6 wherein the loss in β-adrenergic sensitivity results from long-term or excessive administration of a β-adrenergic agonist.

8. The method of claim 7 wherein the β-adrenergic agonist is selected from the group consisting of procaterol, albuterol, salmeterol, formoterol, and doputamine.

9. The method of claim 1 wherein the pathologic change is observed in lung tissue.

10. The method of claim 9 wherein the pathologic change results in a disease or condition benefiting from the improvement of lung function.

11. The method of claim 10 wherein the disease or condition is a bronchoconstrictive disease.

12. The method of claim 10 wherein the disease or condition is selected from the group consisting of emphysema, chronic bronchitis, chronic obstructive pulmonary disease (COPD), pulmonary edema, cystic fibrosis (CF), occlusive lung disease, acute respiratory deficiency syndrome (ARDS), asthma, radiation-induced injury of the lung, and lung injuries resulting from other factors, such as, infectious causes, inhaled toxins, or circulating exogenous toxins, aging and genetic predisposition to impaired lung function.

13. The method of claim 12 wherein the mammalian subject is human.

14. The method of claim 13 wherein the human subject is in need of bronchodilation.

15. The method of claim 1 wherein the pathologic change is observed in cardiac tissue.

16. The method of claim 15 wherein the mammalian subject is human.

17. The method of claim 16 wherein the human subject has been diagnosed with a heart disease.

18. The method of claim 17 wherein the heart disease is chronic or congestive heart failure (CHF).

19. The method of claim 3 wherein the compound is capable of binding to an additional receptor kinase.

20. The method of claim 19 wherein the additional receptor kinase is an activin receptor (Alk4).

21. The method of claim 2 wherein the compound is a small organic molecule.

22. The method of claim 21 wherein the small organic molecule is a compound of formula (1) 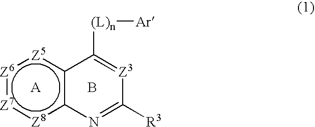
or the pharmaceutically acceptable salts thereof
wherein R³ is a noninterfering substituent;
each Z is CR² or N, wherein no more than two Z positions in ring A are N, and
wherein two adjacent Z positions in ring A cannot be N;
each R² is independently a noninterfering substituent;
L is a linker;
n is 0 or 1; and
Ar′ is the residue of a cyclic aliphatic, cyclic heteroaliphatic, aromatic or heteroaromatic moiety optionally substituted with 1-3 noninterfering substituents.

23. The method of claim 22 wherein the compound is a quinazoline derivative.

24. The method of claim 23 wherein wherein Z³ is N; and Z⁵-Z⁸ are CR².

25. The method of claim 23 wherein Z³ is N; and at least one of Z⁵-Z⁸ is nitrogen.

26. The method of claim 23 wherein R³ is an optionally substituted phenyl moiety

27. The method of claim 26 wherein R³ is selected from the group consisting of 2-, 4-, 5-, 2,4- and 2,5-substituted phenyl moieties.

28. The method of claim 27 wherein at least one substituent of the phenyl moiety is an alkyl(1-6C), or halo.

29. The method of claim 21, wherein the small organic molecule is a compound of formula (2) 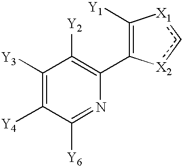
wherein Y₁ is phenyl or naphthyl optionally substituted with one or more substituents selected from halo, alkoxy(1-6C), alkylthio(1-6C), alkyl(1-6C), haloalkyl (1-6C), —O—(CH₂)ₘ-Ph, —S—(CH₂)ₘ-Ph, cyano, phenyl, and CO₂R, wherein R is hydrogen or alkyl(1-6C), and m is 0-3; or phenyl fused with a 5- or 7-membered aromatic or non-aromatic ring wherein said ring contains up to three heteroatoms, independently selected from N, O, and
Y₂, Y₃, Y₄, and Y₅ independently represent hydrogen, alkyl(1-6C), alkoxy(1-6C), haloalkyl(1-6C), halo, NH₂, NH-alkyl(1-6C), or NH(CH₂)ₙ-Ph wherein n is 0-3; or an adjacent pair of Y₂, Y₃, Y₄, and Y₅ form a fused 6-membered aromatic ring optionally containing up to 2 nitrogen atoms, said ring being optionally substituted by one or more substituents independently selected from alkyl(1-6C), alkoxy(a-6C), haloalkyl(1-6C), halo, NH₂, NH-alkyl(1-6C), or NH(CH₂)ₙ-Ph, wherein n is 0-3, and the remainder of Y₂, Y₃, Y₄, and Y₅ represent hydrogen, alkyl(1-6C), alkoxy(1-6C), haloalkyl(1-6C), halo, NH₂, NH-alkyl(1-6C), or NH(CH₂)ₙ-Ph wherein n is 0-3; and
one of X₁ and X₂ is N and the other is NR₆, wherein R₆ is hydrogen or alkyl(1-6C)

30. The method of claim 21 wherein said small organic molecule is a compound of formula (3) 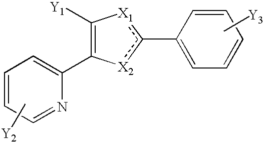
wherein Y₁ is naphthyl, anthracenyl, or phenyl optionally substituted with one or more substituents selected from the group consisting of halo, alkoxy(1-6C), alkylthio(1-6C), alkyl(1-6C), —O—(CH₂)-Ph, —S—(CH₂)ₙ-Ph, cyano, phenyl, and CO₂R, wherein R is hydrogen or alkyl(1-6C), and n is 0, 1, 2, or 3; or Y₁ represents phenyl fused with an aromatic or non-aromatic cyclic ring of 5-7 members wherein said cyclic ring optionally contains up to two heteroatoms, independently selected from N, O, and S;
Y₂ is H, NH(CH₂)ₙ-Ph or NH-alkyl(1-6C), wherein n is 0, 1, 2, or 3;
Y₃ is CO₂H, CONH₂, CN, NO₂, alkylthio(1-6C), —SO₂-alkyl(C1-6), alkoxy(C1-6), SONH₂, CONHOH, NH₂, CHO, CH₂NH₂, or CO₂R, wherein R is hydrogen or alkyl(1-6C); one of X₁ and X₂ is N or CR′, and other is NR′ or CHR′ wherein R′ is hydrogen, OH, alkyl(C-16), or cycloalkyl(C3-7); or when one of X₁ and X₂ is N or CR′ then the other may be S or O.

31. The method of claim 21 wherein said small organic molecule is a compound of formula (4) 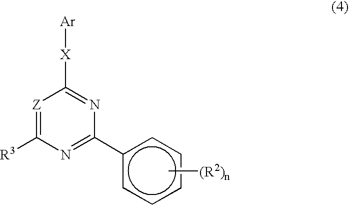
and the pharmaceutically acceptable salts and prodrug forms thereof; wherein
Ar represents an optionally substituted aromatic or optionally substituted heteroaromatic moiety containing 5-12 ring members wherein said heteroaromatic moiety contains one or more O, S, and/or N with a proviso that the optionally substituted Ar is not 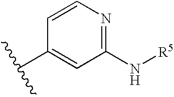
wherein R⁵ is H, alkyl (1-6C), alkenyl (2-6C), alkynyl (2-6C), an aromatic or heteroaromatic moiety containing 5-11 ring members;
X is NR¹, O, or S;
R¹ is H, alkyl (1-8C), alkenyl (2-8C), or alkynyl (2-8C);
Z represents N or CR⁴;
each of R³ and R⁴ is independently H, or a non-interfering substituent;
each R² is independently a non-interfering substituent; and
n is 0, 1, 2, 3, 4, or 5. In one embodiment, if n>2, and the R²'s are adjacent, they can be joined together to form a 5 to 7 membered non-aromatic, heteroaromatic, or aromatic ring containing 1 to 3 heteroatoms where each heteroatom can independently be O, N, or S.

32. A method of claim 21 wherein said small organic molecule is a compound of formula (5) 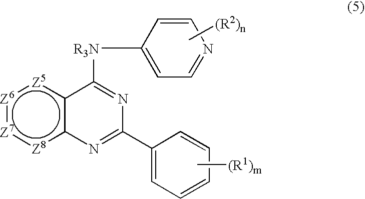
or the pharmaceutically acceptable salts thereof;
wherein each of Z⁵, Z⁶, Z⁷ and Z⁸ is N or CH and wherein one or two Z⁵, Z⁶, Z⁷ and Z⁸ are N and wherein two adjacent Z positions cannot be N;
wherein m and n are each independently 0-3;
wherein two adjacent R¹ groups may be joined to form an aliphatic heterocyclic ring of 5-6 members;
wherein R² is a noninterfering substituent; and
wherein R³ is H or CH₃.

33. A method for counteracting decline in β-adrenergic receptor sensitivity, comprising administering to a mammalian subject in need an effective amount of a compound capable of inhibiting TGF-β signaling through a TGF-β receptor.

34. The method of claim 33 wherein the decline in β-adrenergic receptor sensitivity is agonist-induced.

35. The method of claim 34 wherein the loss in β-adrenergic receptor sensitivity results from one or more causes selected from the group consisting of agonist-induced uncoupling, sequestration, degradation and desensitization of a β-adrenergic receptor.

36. The method of claim 33 wherein the loss in β-adrenergic receptor sensitivity is due to an agonist-independent mechanism.

37. The method of claim 36 wherein the mammalian subject is human.

38. The method of claim 37 wherein the human subject is in need of bronchodilation.

39. The method of claim 38 wherein the human subject has been diagnosed with a disease or condition benefiting from the improvement of lung function.

40. The method of claim 39 wherein the disease or condition benefiting from the improvement of lung function is selected from the group consisting of emphysema, chronic bronchitis, chronic obstructive pulmonary disease (COPD), pulmonary edema, cystic fibrosis, occlusive lung disease, acute respiratory deficiency syndrome (ARDS), asthma, radiation-induced injury of the lung, lung injuries resulting from infectious causes, inhaled toxins, or circulating exogenous toxins, aging and genetic predisposition to impaired lung function.

41. The method of claim 39 wherein the disease or condition benefiting from the improvement of lung function involves acute lung injury.

42. The method of claim 39 wherein the disease or condition benefiting from the improvement of lung function is unaccompanied by lung fibrosis.

43. The method of claim 39 wherein the disease or condition benefiting from the improvement of lung function is at a stage when lung fibrosis is not a major symptom.

44. The method of claim 39 wherein the disease or condition benefiting from the improvement of lung function is associated with inflammation of the lungs.

45. The method of claim 39 wherein the disease or condition benefiting from the improvement of lung function is associated with abnormal inflammatory response of the lungs to noxious particles or gases.

46. The method of claim 39 wherein the disease or condition benefiting from the improvement of lung function is chromic obstructive pulmonary disease (COPD).

47. The method of claim 39 wherein the human subject is treated with a β-adrenergic agonist.

48. The method of claim 47 wherein the β-adrenergic receptor is a β2-adrenergic receptor.

49. The method of claim 48 wherein the β2-adrenergic agonist is a bronchodilator.

50. The method of claim 48 wherein the β2-adrenergic agonist is selected from the group consisting of procaterol, albuterol, salmeterol, and formoterol.

51. The method of claim 37 wherein the mammalian subject has been diagnosed with a heart disease.

52. The method of claim 52 wherein the heart disease is congestive heart failure.

53. The method of claim 52 wherein the administration of the compound capable of inhibiting TGF-β signaling through a TGF-β receptor results in increased ionotropy.

54. The method of claim 52 wherein the administration of the compound capable of inhibiting TGFβ signaling through a TGFβ receptor results in decrease in circulating catecholamines.

55. The method of claim 52 wherein the administration of the compound capable of inhibiting TGFβ signaling through a TGFβ receptor results in decreased arrhythmia and peripheral vasoconstriction.

56. The method of claim 52 wherein the human subject is treated with brain-derived natriuretic peptide (BNP).

57. The method of claim 33 wherein said receptor is a TGFβ-R1 receptor kinase.

58. The method of claim 57 wherein the compound capable of inhibiting TGF-β signaling through said TGFβ-R1 receptor kinase is administered concurrently with treatment with a compound resulting in a loss in β-adrenergic receptor sensitivity.

59. The method of claim 57 wherein the compound capable of inhibiting TGFβ signaling through said TGFβ-R1 receptor kinase is administered intermittently with treatment with a compound resulting in a loss in β-adrenergic receptor sensitivity.

60. The method of claim 57 wherein the compound capable of inhibiting TGFβ signaling through said TGFβ-R1 receptor kinase is administered following treatment with a compound resulting in desensitization of a β-adrenergic receptor.

61. A method for selective inhibition of β2-adrenergic receptor (β2-AR) expression and response to a β-adrenergic receptor antagonist, comprising treating a cell expressing said β2-AR with a compound capable of TGF-β signaling through a TGF-β receptor.

62. The method of claim 61 wherein the TGF-β receptor is a TGFβ-R1 kinase.

63. The method of claim 62 wherein the cell is a cardiac cell.

64. The method of claim 63 wherein the cardiac cell is diseased.

65. The method of claim 64 wherein the cardiac cell is that of a subject having congestive heart failure (CHF).


SEQUENCE LISTING (33 sequences)

SEQ ID NO: 1 - DNA, 20 bp, Artificial Sequence
  primer
cggctaccacatccaaggaa

SEQ ID NO: 2 - DNA, 18 bp, Artificial Sequence
  primer
gctggaattaccgcggct

SEQ ID NO: 3 - DNA, 22 bp, Artificial Sequence
  primer
tgctggcaccagacttgccctc

SEQ ID NO: 4 - DNA, 20 bp, Artificial Sequence
  primer
tgctacaacgaccccaagtg

SEQ ID NO: 5 - DNA, 21 bp, Artificial Sequence
  primer
aggtacacgaaggccatgatg

SEQ ID NO: 6 - DNA, 25 bp, Artificial Sequence
  primer
ccatcgcctcgtccgtagtctcctt

SEQ ID NO: 7 - DNA, 17 bp, Artificial Sequence
  primer
tgccggagcccagattt

SEQ ID NO: 8 - DNA, 23 bp, Artificial Sequence
  primer
attcccataggccttcaaagaag

SEQ ID NO: 9 - DNA, 27 bp, Artificial Sequence
  primer
aggattgccttccaggagcttctgtgc

SEQ ID NO: 10 - DNA, 25 bp, Artificial Sequence
  primer
caactctgccttcaatcctcttatc

SEQ ID NO: 11 - DNA, 23 bp, Artificial Sequence
  primer
tgctagagtagccgttcccatag

SEQ ID NO: 12 - DNA, 27 bp, Artificial Sequence
  primer
aggattgccttccaggagcttctgtgc

SEQ ID NO: 13 - DNA, 26 bp, Artificial Sequence
  primer
cagcacacaataacttggacctacag

SEQ ID NO: 14 - DNA, 21 bp, Artificial Sequence
  primer
aactcgctggttcagctcgta

SEQ ID NO: 15 - DNA, 21 bp, Artificial Sequence
  primer
agccggccttttggtgctcca

SEQ ID NO: 16 - DNA, 18 bp, Artificial Sequence
  primer
tgggctgcatgctcttca

SEQ ID NO: 17 - DNA, 21 bp, Artificial Sequence
  primer
gcggtcaatctcatgcttgtc

SEQ ID NO: 18 - DNA, 21 bp, Artificial Sequence
  primer
ccttccggcagcacaagacca

SEQ ID NO: 19 - DNA, 20 bp, Artificial Sequence
  primer
accgccaatgccatagactt

SEQ ID NO: 20 - DNA, 18 bp, Artificial Sequence
  primer
caccttcagcgccacctt

SEQ ID NO: 21 - DNA, 21 bp, Artificial Sequence
  primer
cccagtgccctgagcatgcga

SEQ ID NO: 22 - DNA, 19 bp, Artificial Sequence
  primer
gcctgtcccgcagtatcgt

SEQ ID NO: 23 - DNA, 23 bp, Artificial Sequence
  primer
gaacacaagcagaaccgagaaga

SEQ ID NO: 24 - DNA, 20 bp, Artificial Sequence
  primer
cacgggtgcacagcacggct

SEQ ID NO: 25 - DNA, 20 bp, Artificial Sequence
  primer
cgggagtaccagctgaacga

SEQ ID NO: 26 - DNA, 22 bp, Artificial Sequence
  primer
tgggttgggatgtaatttggtt

SEQ ID NO: 27 - DNA, 31 bp, Artificial Sequence
  primer
cggcgtactacctgaatgacttggacagaat

SEQ ID NO: 28 - DNA, 17 bp, Artificial Sequence
  primer
tgcggacccgtgtgaag

SEQ ID NO: 29 - DNA, 18 bp, Artificial Sequence
  primer
cgctgaccacccacatca

SEQ ID NO: 30 - DNA, 28 bp, Artificial Sequence
  primer
aggcatcgtcgaaacacacttcaccttc

SEQ ID NO: 31 - DNA, 30 bp, Artificial Sequence
  primer
gcttcatattacctaaatgatttggataga

SEQ ID NO: 32 - DNA, 25 bp, Artificial Sequence
  primer
ccacaatgcctgtagtcttcactct

SEQ ID NO: 33 - DNA, 29 bp, Artificial Sequence
  primer
tcccagaccaactacattccaactcagca